SEQUENCE LISTING (61 sequences)

SEQ ID NO: 1 - DNA, 1410 bp, Aeromonas hydrophila
  CDS (1)...(1410)
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaa

SEQ ID NO: 2 - protein, 470 aa, Aeromonas hydrophila
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 3 - DNA, 1482 bp, Aeromonas hydrophila
atgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaataa

SEQ ID NO: 4 - protein, 493 aa, Aeromonas hydrophila
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 5 - DNA, 1541 bp, Clostridium septicum
tgttaataatatgttaatattttgataacatttattatataataaattatttattttaaaattaaagggagggatatttatgtcaaaaaaatcttttgctaaaaaagtaatttgtacatctatgattgcaattcagtgtgcggcagtagtaccacatgtacaagcttatgcacttacaaatcttgaagaggggggatatgcaaatcataataatgcttcttcaattaaaatatttggatatgaagacaatgaagatttaaaagctaaaattattcaagatccagagtttataagaaattgggcaaatgtagctcattcattaggatttggatggtgcggtggaacggctaatccaaacgttggacaaggttttgaatttaaaagagaagttggggcaggtggaaaagtatcttatttattatctgctagatacaatccaaatgatccttatgcaagtggatatcgtgcaaaagatagactttctatgaaaatatcaaatgttagatttgttattgataatgattctataaaattaggtacacctaaagtgaaaaaattagcacctttaaactctgctagttttgatttaaaaatgaaagtaaaactgagtctaaattatcaaaaacatttaattatacaacttctaaaacagtttctaaaacagataactttaaatttggagaaaaaataggagtaaaaacatcatttaaagtaggtcttgaagctatagctgacagtaaagttgagacaagctttgaatttaatgcagaacaaggttggtcaaatacaaatagtactactgaaactaaacaagaaagtactacatatactgcaacagtttctccacaaactaaaaagagattattcctagatgtgttaggatcacaaattgatattccttatgaaggaaaaatatatatggaatacgacatagaattaatgggatttttaagatatacaggaaatgctcgtgaagatcatactgaagatagaccaacagttaaacttaaatttggtaaaaacggtatgagtgctgaggaacatcttaaagatttatatagtcataagaatattaatggatattcagaatgggattggaaatgggtagatgagaaatttggttatttatttaaaaattcatacgatgctcttactagtagaaaattaggaggaataataaaaggctcatttactaacattaatggaacaaaaatagtaattagagaaggtaaagaaattccacttcctgataagaagagaagaggaaaacgttcagtagattctttagatgctagattacaaaatgaaggtattagaatagaaaatattgaaacacaagatgttccaggatttagactaaatagcataacatacaatgataaaaaattgatattaattaataatatataattataatttattaaaatatgcttctctatactttatattaatatttaaagtataaaaactaacaaaatctcacttagtaggtagaattgtataaaaacaaatctacctactatttttttattatttagtcg

SEQ ID NO: 6 - protein, 443 aa, Clostridium septicum
MSKKSFAKKVICTSMIAIQCAAVVPHVQAYALTNLEEGGYANHNNASSIKIFGYEDNEDLKAKIIQDPEFIRNWANVAHSLGFGWCGGTANPNVGQGFEFKREVGAGGKVSYLLSARYNPNDPYASGYRAKDRLSMKISNVRFVIDNDSIKLGTPKVKKLAPLNSASFDLINESKTESKLSKTFNYTTSKTVSKTDNFKFGEKIGVKTSFKVGLEAIADSKVETSFEFNAEQGWSNTNSTTETKQESTTYTATVSPQTKKRLFLDVLGSQIDIPYEGKIYMEYDIELMGFLRYTGNAREDHTEDRPTVKLKFGKNGMSAEEHLKDLYSHKNINGYSEWDWKWVDEKFGYLFKNSYDALTSRKLGGIIKGSFTNINGTKIVIREGKEIPLPDKKRRGKRSVDSLDARLQNEGIRIENIETQDVPGFRLNSITYNDKKLILINNI

SEQ ID NO: 7 - DNA, 1332 bp, Clostridium septicum
atgtcaaaaaaatcttttgctaaaaaagtaatttgtacatctatgattgcaattcagtgtgcggcagtagtaccacatgtacaagcttatgcacttacaaatcttgaagaggggggatatgcaaatcataataatgcttcttcaattaaaatatttggatatgaagacaatgaagatttaaaagctaaaattattcaagatccagagtttataagaaattgggcaaatgtagctcattcattaggatttggatggtgcggtggaacggctaatccaaacgttggacaaggttttgaatttaaaagagaagttggggcaggtggaaaagtatcttatttattatctgctagatacaatccaaatgatccttatgcaagtggatatcgtgcaaaagatagactttctatgaaaatatcaaatgttagatttgttattgataatgattctataaaattaggtacacctaaagtgaaaaaattagcacctttaaactctgctagttttgatttaataaatgaaagtaaaactgagtctaaattatcaaaaacatttaattatacaacttctaaaacagtttctaaaacagataactttaaatttggagaaaaaataggagtaaaaacatcatttaaagtaggtcttgaagctatagctgacagtaaagttgagacaagctttgaatttaatgcagaacaaggttggtcaaatacaaatagtactactgaaactaaacaagaaagtactacatatactgcaacagtttctccacaaactaaaaagagattattcctagatgtgttaggatcacaaattgatattccttatgaaggaaaaatatatatggaatacgacatagaattaatgggatttttaagatatacaggaaatgctcgtgaagatcatactgaagatagaccaacagttaaacttaaatttggtaaaaacggtatgagtgctgaggaacatcttaaagatttatatagtcataagaatattaatggatattcagaatgggattggaaatgggtagatgagaaatttggttatttatttaaaaattcatacgatgctcttactagtagaaaattaggaggaataataaaaggctcatttactaacattaatggaacaaaaatagtaattagagaaggtaaagaaattccacttcctgataagaagagaagaggaaaacgttcagtagattctttagatgctagattacaaaatgaaggtattagaatagaaaatattgaaacacaagatgttccaggatttagactaaatagcataacatacaatgataaaaaattgatattaattaataatatataa

SEQ ID NO: 8 - protein, 443 aa, Clostridium septicum; identical to SEQ ID NO: 6

SEQ ID NO: 9 - protein, 116 aa, Artificial Sequence
  AFAI antibody fragment
VFDVQLQASGGGVVQPGGSLRLSCAAHDPIFDKNLMGWGRQRPGKQREYVATISGNGGTNYASSVEGRFTISRDNAKKTVYLQMNDLKPEDTAVYYCNSAFAIWGQGIQVTVSSVH

SEQ ID NO: 10 - DNA, 27 bp, Artificial Sequence
  Primer
gcggctgacagcagtgggcgtcgtgct

SEQ ID NO: 11 - DNA, 28 bp, Artificial Sequence
  Primer
agcacgacgcccactgctgtcagccgcg

SEQ ID NO: 12 - DNA, 31 bp, Artificial Sequence
  Primer
agcagtgggcgtagcgctcaaagtgtggacg

SEQ ID NO: 13 - DNA, 31 bp, Artificial Sequence
  Primer
gtccacactttgagcgctacgcccactgctg

SEQ ID NO: 14 - DNA, 40 bp, Artificial Sequence
  Primer
gtctcctcagtgcacctagtccctgcagagcccgtctatc

SEQ ID NO: 15 - DNA, 40 bp, Artificial Sequence
  Primer
atagacgggctctgcagggactaggtgcactgaggagacg

SEQ ID NO: 16 - DNA, 40 bp, Artificial Sequence
  Primer
gtgcacctagtccctcgtggttccgcagagcccgtctatc

SEQ ID NO: 17 - DNA, 41 bp, Artificial Sequence
  Primer
atagacgggctctgcggaaccacgagggactaggtgcactg

SEQ ID NO: 18 - DNA, 40 bp, Artificial Sequence
  Primer
gtctcctcagtgcactcaggccgtagtgctcaagcagagc

SEQ ID NO: 19 - DNA, 52 bp, Artificial Sequence
  Primer
ctggatagacgggctctgcttgagcactacggcctgagtgcactgaggagac

SEQ ID NO: 20 - DNA, 1500 bp, Artificial Sequence
  proaerolysin with a uPA cleavage site in the activation sequence
atgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcagtgggcgtagcgctcaaagtgtggacggcgctggtcaaggcctgaggctggagatcccgctcgatgcgcaagagctctccgggcttggcttcaacaacgtcagcctcagcgtgacccctgctgccaatcaacatcatcatcatcatcattaa

SEQ ID NO: 21 - protein, 499 aa, Artificial Sequence
  proaerolysin with a uPA cleavage site in the activation sequence
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALEIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSSGRSAQSVDGAGQGLRLEIPLDAQELSGLGFNNVSLSVTPAANQHHHHHH

SEQ ID NO: 22 - DNA, 1842 bp, Artificial Sequence
  proaerolysin with a uPA cleavage site in the activation sequence, and an AFAI antibody fragment as a targeting unit
atgaaaaaaaccgctatcgcgatcgcagttgcactggctggtttcgctaccgttgcgcaggccgtcttcgatgtgcagctgcaggcgtctggaggaggcgtggtgcagcctggggggtctctgagactctcctgtgcagcccatgatcccatcttcgataagaatctcatgggctggggccgccaggctccaggaaagcagcgcgaatatgtcgcgactattagtggtaatggtggaacaaattatgcaagctccgttgagggccgattcaccatctctagagacaacgccaagaaaacggtgtatctgcaaatgaacgacctgaaacctgaggacacggccgtctattactgtaactcagcttttgctatctggggccagggcatccaggtcaccgtctcctcagtgcacgcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcagtgggcgtagcgctcaaagtgtggacggcgctggtcaaggcctgaggctggagatcccgctcgatgcgcaagagctctccgggcttggcttcaacaacgtcagcctcagcgtgacccctgctgccaatcaacatcatcatcatcatcattga

SEQ ID NO: 23 - protein, 613 aa, Artificial Sequence
  proaerolysin with a uPA cleavage site in the activation sequence, and an AFAI antibody fragment as a targeting unit
MKKTAIAIAVALAGFATVAQAVFDVQLQASGGGVVQPGGSVRLSCAAHDPIFDKNLMGWGRQAPGKQREYVATISGNGGTNYASSVEGRFTISRDNAKKTVYLQMNDLKPEDTAVYYCNSAFAIWGQGIQVTVSSVHAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSSGRSAQSVDGAGQGLRLEIPLDAQELSGLGFNNVSLSVTPAANQHHHHHH

SEQ ID NO: 24 - DNA, 1860 bp, Artificial Sequence
  proaerolysin with a uPA cleavage site in the activation sequence, and an AFAI antibody fragment as a targeting unit, attached via a uPA-cleavable linker
atgaaaaaaaccgctatcgcgatcgcagttgcactggctggtttcgctaccgttgcgcaggccgtcttcgatgtgcagctgcaggcgtctggaggaggcgtggtgcagcctggggggtctctgagactctcctgtgcagcccatgatcccatcttcgataagaatctcatgggctggggccgccaggctccaggaaagcagcgcgaatatgtcgcgactattagtggtaatggtggaacaaattatgcaagctccgttgagggccgattcaccatctctagagacaacgccaagaaaacggtgtatctgcaaatgaacgacctgaaacctgaggacacggccgtctattactgtaactcagcttttgctatctggggccagggcatccaggtcaccgtctcctcagtgcactcaggccgtagtgctcaagcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcagtgggcgtagcgctcaaagtgtggacggcgctggtcaaggcctgaggctggagatcccgctcgatgcgcaagagctctccgggcttggcttcaacaacgtcagcctcagcgtgacccctgctgccaatcaacatcatcatcatcatcattga

SEQ ID NO: 25 - protein, 619 aa, Artificial Sequence
  proaerolysin with a uPA cleavage site in the activation sequence, and an AFAI antibody fragment as a targeting unit, attached via a uPA-cleavable linker
MKKTAIAIAVALAGFATVAQAVFDVQLQASGGGVVQPGGSVRLSCAAHDPIFDKNLMGWGRQAPGKQREYVATISGNGGTNYASSVEGRFTISRDNAKKTVYLQMNDLKPEDTAVYYCNSAFAIWGQGIQVTVSSVHSGRSAQAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSSGRSAQSVDGAGQGLRLEIPLDAQELSGLGFNNVSLSVTPAANQHHHHHH

SEQ ID NO: 26 - DNA, 35 bp, Artificial Sequence
  primer
ctcgcggctgacagccatccggtgcgtgctcgcag

SEQ ID NO: 27 - DNA, 35 bp, Artificial Sequence
  primer
ctgcgagcacgcaccggatggctgtcagccgcgag

SEQ ID NO: 28 - DNA, 36 bp, Artificial Sequence
  primer
gacagccatccggtgggcctgctcagtgtggacggc

SEQ ID NO: 29 - DNA, 36 bp, Artificial Sequence
  primer
gccgtccacactgagcaggcccaccggatggctgtc

SEQ ID NO: 30 - DNA, 33 bp, Artificial Sequence
  primer
ccggtgggcctggtcgctcgcagtgtggacggc

SEQ ID NO: 31 - DNA, 33 bp, Artificial Sequence
  primer
gccgtccacactgcgagcgagcaggcccaccgg

SEQ ID NO: 32 - DNA, 36 bp, Artificial Sequence
  primer
ggcctgctcgctcgcggcggctcaagtgtggacggc

SEQ ID NO: 33 - DNA, 36 bp, Artificial Sequence
  primer
gccgtccacacttgagccgccgcgagcgagcaggcc

SEQ ID NO: 34 - DNA, 33 bp, Artificial Sequence
  primer
gctcgcggcggctcaggccgtagtgtggagggc

SEQ ID NO: 35 - DNA, 33 bp, Artificial Sequence
  primer
gccgtccacactacggcctgagccgccgcgagc

SEQ ID NO: 36 - DNA, 33 bp, Artificial Sequence
  primer
ggctcaggccgtagtgcgcaaagtgtggacggc

SEQ ID NO: 37 - DNA, 33 bp, Artificial Sequence
  primer
gccgtccacactttgcgcactacggcctgagcc

SEQ ID NO: 38 - DNA, 1506 bp, Artificial Sequence
  proaerolysin with an MMP2 cleavage site in the activation sequence
atgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccatccggtgggcctgctcgctcgcagtgtggacggcgctggtcaaggcctgaggctggagatcccgctcgatgcgcaagagctctccgggcttggcttcaacaacgtcagcctcagcgtgacccctgctgccaatcaacatcatcatcatcatcattaa

SEQ ID NO: 39 - protein, 501 aa, Artificial Sequence
  proaerolysin with an MMP2 cleavage site in the activation sequence
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALEIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHPVGLLARSVDGAGQGLRLEIPLDAQELSGLGFNNVSLSVTPAANQHHHHHH

SEQ ID NO: 40 - DNA, 1530 bp, Artificial Sequence
  proaerolysin with an MMP2 cleavage site and a uPA cleavage  site in the activation sequence
atgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccatccggtgggcctgctcgctcgcggcggctcaggccgtagtgcgcaaagtgtggacggcgctggtcaaggcctgaggctggagatcccgctcgatgcgcaagagctctccgggcttggcttcaacaacgtcagcctcagcgtgacccctgctgccaatcaacatcatcatcatcatcattaa

SEQ ID NO: 41 - protein, 509 aa, Artificial Sequence
  proaerolysin with an MMP2 cleavage site and a uPA cleavage  site in the activation sequence
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALEIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHPVGLLARGGSGRSAQSVDGAGQGLRLEIPLDAQELSGLGFNNVSLSVTPAANQHHHHHH

SEQ ID NO: 42 - DNA, 1500 bp, Artificial Sequence
  proaerolysin with R336A mutation to large lobe binding domain
atgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaacgcaccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaacatcatcatcatcatcattga

SEQ ID NO: 43 - protein, 499 aa, Artificial Sequence
  proaerolysin with R336A mutation to large lobe binding domain
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQHHHHHH

SEQ ID NO: 44 - DNA, 1842 bp, Artificial Sequence
  proaerolysin with R336A mutation to large lobe binding domain, and AFAI as an artificial regulatory domain
atgaaaaaaaccgctatcgcgatcgcagttgcactggctggtttcgctaccgttgcgcaggccgtcttcgatgtgcagctgcaggcgtctggaggaggcgtggtgcagcctggggggtctctgagactctcctgtgcagcccatgatcccatcttcgataagaatctcatgggctggggccgccaggctccaggaaagcagcgcgaatatgtcgcgactattagtggtaatggtggaacaaattatgcaagctccgttgagggccgattcaccatctctagagacaacgccaagaaaacggtgtatctgcaaatgaacgacctgaaacctgaggacacggccgtctattactgtaactcagcttttgctatctggggccagggcatccaggtcaccgtctcctcagtgcacgcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctcgagattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaacgcaccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaacatcatcatcatcatcattga

SEQ ID NO: 45 - protein, 613 aa, Artificial Sequence
  proaerolysin with R336A mutation to large lobe binding domain, and AFAI as an artificial regulatory domain
MKKTAIAIAVALAGFATVAQAVFDVQLQASGGGVVQPGGSLRLSCAAHDPIFDKNLMGWGRQAPGKQREYVATISGNGGTNYASSVEGRFTISRDNAKKTVYLQMNDLKPEDTAVYYCNSAFAIWGQGIQVTVSSVHAEPVYPDQLRLFSLGQGVCGDKYRPVNREEAQSVKSNIVGMMGQWQISGLANGWVIMGPGYNGEIKPGTASNTWCYPTNPVTGEIPTLSALEIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNAPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQHHHHHH

SEQ ID NO: 46 - protein, 6 aa, Artificial Sequence
  Caspase 1 cleavage site
YVADIX

SEQ ID NO: 47 - protein, 4 aa, Artificial Sequence
  tissue-type plasminogen activator cleavage site
FGRX

SEQ ID NO: 48 - protein, 10 aa, Artificial Sequence
  matrx metalloprotease 14 cleavage site
GGPLGLYAGG

SEQ ID NO: 49 - protein, 7 aa, Artificial Sequence
  human glandular kallikrein 2 cleavage site
GKAFRRX

SEQ ID NO: 50 - protein, 6 aa, Artificial Sequence
  thrombin cleavage site
LVPRGS

SEQ ID NO: 51 - protein, 6 aa, Artificial Sequence
  urokinase-type plasminagen activator cleavage site
SGRSAQ

SEQ ID NO: 52 - protein, 8 aa, Artificial Sequence
  matrix metalloprotease 2 cleavage site
HPVGLLAR

SEQ ID NO: 53 - DNA, 31 bp, Artificial Sequence
  primer
atagacgggctctgcgtgcactgaggagacg

SEQ ID NO: 54 - DNA, 31 bp, Artificial Sequence
  primer
gtctcctcagtgcacgcagagcccgtctatc

SEQ ID NO: 55 - DNA, 27 bp, Artificial Sequence
  primer
cacccggacaacgcaccgaactggaac

SEQ ID NO: 56 - DNA, 27 bp, Artificial Sequence
  primer
gttccagttcggtgcgttgtccgggtg

SEQ ID NO: 57 - protein, 4 aa, Artificial Sequence
  Factor Xa cleavage site
IEGR

SEQ ID NO: 58 - protein, 5 aa, Artificial Sequence
  Enterokinase cleavage site
DDDDK

SEQ ID NO: 59 - protein, 5 aa, Artificial Sequence
  Thrombin cleavage site
LVPRG

SEQ ID NO: 60 - DNA, 32 bp, Artificial Sequence
  Primer
cacccggacaactgtccgaactggaaccacac

SEQ ID NO: 61 - DNA, 34 bp, Artificial Sequence
  Primer
ggttccagttcggacagttgtccgggtgggtaaa

(19) United States
(12) Patent Application Publication
(10) Pub. No.: US 2009/0016988 A1
(43) Pub. Date: Jan. 15, 2009

(54) Modified Pore-Forming Protein Toxins and Use Thereof

(76) Inventor: J. Thomas Buckley, Victoria (CA)

Correspondence Address: KLARQUIST SPARKMAN, LLP, 121 SW SALMON STREET, SUITE 1600, PORTLAND, OR, 97204, US

(21) Appl. No.: 12/094,597

(22) PCT Filed: Nov. 21, 2006
(86) PCT No.: PCT/CA06/01900

§ 371 Date: Aug. 22, 2008

Related U.S. Application Data
(60) Provisional application No. 60/738,916, filed on Nov. 21, 2005.

(51) Int. Cl. A61K 38/20; C07K 14/33; C07K 14/195; A61K 39/08; A61K 39/02; C07K 16/46; C07K 2/00; C07K 14/62; A61P 35/00; C12N 9/00; C07H 21/04; C12N 15/63; C12N 5/10; A61K 31/711; C07K 14/55

(52) U.S. Cl. 424/85.2; 530/324; 424/239.1; 424/236.1; 530/391.7; 530/303; 435/183; 530/300; 536/23.7; 435/320.1; 435/325; 514/44; 530/351

(57) ABSTRACT

The present invention provides modified pore-forming protein toxins (MPPTs), capable of being used to kill cancer cells. The MPPTs according to the present invention comprise a modification of the naturally occurring activation sequence comprising one or more general cleavage sites, each of which is cleavable by general activating agent, or a plurality of specific cleavage sites, each of which is cleavable by a specific activating agent. Optional further modifications that allow specific targeting of these molecules are also described. These MPPTs may be used to treat cancer.

### FIELD OF THE INVENTION

The present invention relates to the field of modified pore-forming protein toxins and their use to kill cancer cells.

### BACKGROUND

Many cytolytic proteins have been described (Lesieur et al. Mol. Membr. Biol. 14:45064, 1997). These naturally occurring cytotoxic proteins include mammalian proteins such as perforin, and bacterial proteins such as aerolysin (produced by Aeromonas hydrophila), α-hemolysin (produced by Staphylococcus aureus), alpha toxin (produced by Clostridium septicum), and δ-toxin (produced by Bacillus thuringiensis), anthrax protective antigen, Vibrio cholerae VCC toxin, Staphylococcus leucocidins, LSL toxin from Laetiporus sulphureus, epsilon toxin from Clostridium perfringens, and hydralysins produced by Cnidaria spp.

Some of these cytotoxic proteins are synthesized as inactive protoxins, for example proaerolysin and alpha toxin. These protoxins contain discrete functionalities including a binding domain, which allows binding of the protoxin to a cell, a toxin domain, and either an N-terminal or a C-terminal inhibitory peptide domain that contains a protease cleavage site. Cleavage of the inhibitory peptide domain at the protease cleavage site results in activation of the protoxin, leading to oligomerization of the cytotoxin and insertion into the plasma membrane, producing pores that lead to rapid cytolytic cell death (Rossjohn et al. J. Struct. Biol. 121:92-100, 1998). Pore formation disrupts the cell membranes, and results in death of cells in all phases of the cell cycle, including non-proliferating cells (i.e. Go arrested). These cytotoxins are not specific in the type of cells they are able to kill, as their binding domains target molecules that are found on most cells, and they may be activated by proteases that are not cell-specific.

Cancer is characterized by an increase in the number of abnormal, or neoplastic cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. Many strategies for developing therapeutics for the treatment of cancer have focused on taking advantage of the differences in gene expression between normal cells and cancer cells, and targeting cancer cells using molecular markers that are specific to cancer cells.

Cytolytic pore-forming proteins or modified versions of these proteins have been proposed as potential therapeutics for the treatment of cancer. For example, U.S. Pat. No. 5,777,078 describes pore-forming agents that are activated at the surface of a cell by a number of conditions, including proteolysis, to lyse the cell. These pore-forming agents can be used generally to destroy unwanted cells associated with a pathological condition in an animal. WO 98/020135 describes methods and compositions relating to Pseudomonas exotoxin proproteins modified for selective toxicity. The exotoxin is modified to be activated by a desired protease by insertion of a protease activatable sequence in the proprotein. In one example the exotoxin is modified to insert a prostate specific antigen (PSA) cleavage site for the purpose of targeting and killing prostate cancer cells. In another example, the exotoxin is modified to include a urokinase activatable sequence. U.S. Patent Application No. 2004/0235095 describes the use of modified cytolytic proteins, in particular proaerolysin, for the treatment of prostate cancer. The cytolytic proteins can be modified to include a prostate-specific cleavage site, and/or a prostate-specific binding domain and can be used to selectively target and kill prostate cancer cells.

Modification of cytolytic peptides to include an inhibitory or targeting domain have been described. U.S. Patent Application No. 2002/0045736 describes the modification of cytotoxic peptides, including proaerolysin and homologs, via attachment of an inhibitory molecule that acts to inhibit formation of the active conformation of the cytolytic peptide. This application also describes attachment of targeting molecules to the cytolytic peptide, or molecules that can act as both a targeting molecule and an inhibitory molecule. These inhibitory and/or targeting molecules are attached to the cytolytic peptide via a linker that may or may not be cleavable. U.S. Pat. No. 4,867,973 describes antibody therapeutic enzyme conjugates in which the antibody is linked to a therapeutic agent via a protease cleavable linker. The therapeutic agent may include toxins or fragments of toxins. In addition, International Patent Application No. PCT/US94/04016 (WO 94/25616) describes a chimeric compound active at a cell surface having a delivery component, for example, an antibody or other ligands binding specifically to a target cell that is linked to a pore-forming component such as aerolysin. This application also describes modifications designed to inactivate the pore-forming agent, which can then be specifically activated by a cell-associated substance or condition. Finally, International Patent Application No. PCT/CA2004/000309 (WO 2004/078097) describes peptides including aerolysin or an aerolysin homolog, linked to an agent that specifically binds to a lung cancer cell, as well as nucleic acids that encode such peptides. Methods of using these peptides and nucleic acid sequences to treat lung cancer are also described.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide modified pore forming protein toxins and use thereof. In accordance with an aspect of the present invention, there is provided a broad-spectrum anti-cancer agent comprising a modified pore forming protein toxin, said modified pore forming protein toxin derived from a naturally occurring aerolysin-related pore forming protein and comprising a modified activation sequence in which a native protease cleavage site has been functionally deleted and replaced with: one or more general cleavage sites, each cleavable by an enzyme associated with a plurality of cancers, or two or more specific cleavage sites, each cleavable by an enzyme associated with the presence of a specific cancer, wherein cleavage of said modified activation sequence provides an activated pore forming protein toxin capable of killing cancer cells.

In accordance with another aspect of the invention, there is provided an isolated polynucleotide encoding a broad-spectrum anti-cancer agent according to the present invention.

In accordance with another aspect of the invention, there is provided a vector comprising the polynucleotide encoding a broad-spectrum anti-cancer agent according to the present invention, wherein the polynucleotide is operatively linked to one or more expression control sequences.

In accordance with another aspect of the invention, there is provided a host cell comprising the vector comprising a polynucleotide encoding the broad-spectrum anti-cancer agent according to the present invention.

In accordance with a further aspect of the invention, there is provided a modified pore forming protein toxin derived from proaerolysin and comprising a modified activation sequence in which a native protease cleavage site has been functionally deleted and replaced with one or more general cleavage sites cleavable by urokinase-type plasminogen activator or matrix metalloprotease 2, said modified pore forming protein toxin comprising an amino acid sequence substantially identical to the sequence as set forth in any one of SEQ ID NOs: 21, 23, 25, 39, or 41.

In accordance with still another aspect of the invention, there is provided a pharmaceutical composition comprising a broad-spectrum anti-cancer agent according to the present invention.

In accordance with another aspect of the invention, there is provided a pharmaceutical composition comprising the isolated polynucleotide encoding a broad-spectrum anti-cancer agent according to the present invention.

In accordance with another aspect of the invention, there is provided a pharmaceutical composition comprising a modified pore forming protein toxin according to the present invention.

In accordance with yet another aspect of the invention, there is provided a broad-spectrum anti-cancer agent according to the present invention, for use in decreasing the size of a tumor in a subject.

In accordance with another aspect of the invention, there is provided a broad-spectrum anti-cancer agent according to the present invention, for use in the treatment of cancer in a subject.

In accordance with another aspect of the invention, there is provided a use of a modified pore forming protein toxin in the preparation of a medicament for decreasing the size of a tumor in a subject, said modified pore forming protein toxin derived from a naturally occurring aerolysin-related pore forming protein and comprising a modified activation sequence in which a native protease cleavage site has been functionally deleted and replaced with: one or more general cleavage sites, each cleavable by an enzyme associated with a plurality of cancers, or two or more specific cleavage sites each cleavable by an enzyme associated with the presence of a specific cancer, wherein cleavage of said modified activation sequence provides an activated pore forming protein toxin capable of killing cancer cells.

In accordance with another aspect of the invention, there is provided a use of a modified pore forming protein toxin in the preparation of a medicament for the treatment of cancer in a subject, said modified pore forming protein toxin derived from a naturally occurring aerolysin-related pore forming protein and comprising a modified activation sequence in which a native protease cleavage site has been functionally deleted and replaced with: one or more general cleavage sites each cleavable by an enzyme associated with a plurality of cancers, or two or more specific cleavage sites each cleavable by an enzyme associated with the presence of a specific cancer, wherein cleavage of said modified activation sequence provides an activated pore forming protein toxin capable of killing cancer cells.

In accordance with another aspect of the invention, there is provided a method of decreasing the size of a tumor comprising administering to a subject having cancer an effective amount of a broad-spectrum anti-cancer agent according to the present invention.

In accordance with another aspect of the invention, there is provided a method of treating cancer comprising administering to a subject having cancer an effective amount of a broad-spectrum anti-cancer agent according to the present invention.

In accordance with another aspect of the invention, there is provided a method of preparing a broad-spectrum anti-cancer agent, said method comprising: providing a native pore forming protein toxin wherein said native pore forming protein toxin is proaerolysin or Clostridium septicum alpha toxin; and modifying the activation sequence of said native pore forming protein toxin such that a native protease cleavage site is functionally deleted and replaced by one or more general cleavage sites each cleavable by an enzyme associated with a plurality of cancers, or by two or more specific cleavage sites each cleavable by an enzyme associated with the presence of a specific cancer, wherein cleavage of said modified activation sequence provides an activated pore forming protein toxin capable of killing cancer cells.

### BRIEF DESCRIPTION OF THE FIGURES

FIG. 1 depicts the nucleotide sequence of proaerolysin from Aeromonas hydrophila (SEQ ID NO:1).

FIG. 2 depicts the amino acid sequence of proaerolysin from Aeromonas hydrophila (SEQ ID NO:2).

FIG. 3 depicts the nucleotide sequence of preproaerolysin from Aeromonas hydrophila, including the signal sequence (SEQ ID NO:3).

FIG. 4 depicts the amino acid sequence of preproaerolysin from Aeromonas hydrophila, including the signal sequence (underlined) (SEQ ID NO:4).

FIG. 5 depicts the nucleotide sequence of alpha toxin from Clostridium septicum (SEQ ID NO:5).

FIG. 6 depicts the amino acid sequence of alpha toxin from Clostridium septicum (SEQ ID NO:6).

FIG. 7 depicts the nucleotide sequence of alpha toxin from Clostridium septicum, including the signal sequence (underlined) (SEQ ID NO:7).

FIG. 8 depicts the amino acid sequence of alpha toxin from Clostridium septicum, including the signal sequence (underlined). (SEQ ID NO:8)

FIG. 9 depicts the amino acid sequence of the AFAI antibody fragment (SEQ ID NO:9).

FIG. 10 depicts the nucleotide sequence of a modified proaerolysin (MPPT1; SEQ ID NO:20) in accordance with one embodiment of the present invention having a modified activation sequence comprising a uPA cleavage site and further comprising a His-tag.

FIG. 11 depicts the amino acid sequence of a modified proaerolysin (MPPT1; SEQ ID NO:21) in accordance with one embodiment of the present invention having a modified activation sequence comprising a uPA cleavage site and further comprising a His-tag.

FIG. 12 depicts the nucleotide sequence of a modified proaerolysin (MPPT2; SEQ ID NO:22) in accordance with one embodiment of the present invention having a modified activation sequence comprising a uPA cleavage site, an AFAI targeting unit and further comprising a His-tag.

FIG. 13 depicts the nucleotide sequence of a modified proaerolysin (MPPT3; SEQ ID NO:24) in accordance with one embodiment of the present invention having a modified activation sequence comprising a uPA cleavage site, an AFAI targeting unit attached via a linker comprising a uPA cleavage site and further comprising a His-tag.

FIG. 14 depicts the amino acid sequence of a modified proaerolysin (MPPT2; SEQ ID NO:23) in accordance with one embodiment of the present invention having a modified activation sequence comprising a uPA cleavage site, an AFAI targeting unit and further comprising a His-tag.

FIG. 15 depicts the amino acid sequence of a modified proaerolysin (MPPT3; SEQ ID NO:25) in accordance with one embodiment of the present invention having a modified activation sequence comprising a uPA cleavage site, an AFAI targeting unit attached via a linker comprising a uPA cleavage site and further comprising a His-tag.

FIG. 16 depicts an SDS-PAGE gel showing the results of digestion by uPA of a modified proaerolysin (MPPT1) in accordance with one embodiment of the present invention.

FIG. 17 depicts a SDS-PAGE gel showing the results of digestion of two modified proaerolysins MPPT2 and MPPT3 by uPA.

FIG. 18 depicts the toxicity of the modified proaerolysins MPPT1, MPPT2, and MPPT3 against A549 cells.

FIG. 19 depicts the effect of in vivo administration of the modified proaerolysins MPPT1 and MPPT2 on body weight in mice.

FIG. 20 depicts the effect of the modified proaerolysins MPPT1 and MPPT2 on rate of tumor growth in mice.

FIG. 21 depicts an SDS-PAGE gel showing the cleavage of the modified proaerolysin MPPT1 by uPA.

FIG. 22 depicts the ability of the modified proaerolysin MPPT1 to kill HeLa human cervical cancer cells.

FIG. 23 depicts the ability of the modified proaerolysin MPPT1 to kill A2058 human melanoma cells.

FIG. 24 depicts the ability of the modified proaerolysin MPPT1 to kill EL4 mouse lymphoma cells.

FIG. 25 depicts the nucleotide sequence of a modified proaerolysin (MPPT4; SEQ ID NO:38) in accordance with one embodiment of the present invention having a modified activation sequence comprising a MMP2 cleavage site.

FIG. 26 depicts the amino acid sequence of a modified proaerolysin (MPPT4; SEQ ID NO:39) in accordance with one embodiment of the present invention having a modified activation sequence comprising a MMP2 cleavage site.

FIG. 27 depicts a silver stained SDS-PAGE gel showing the cleavage of proaerolysin and the modified proaerolysin MPPT4 by MMP2.

FIG. 28 depicts a Western blot showing the cleavage of proaerolysin and the modified proaerolysin MPPT4 by MMP2.

FIG. 29 depicts cell killing curves for HT 1080 human fibrosarcoma cells treated with proaerolysin or the modified proaerolysin MPPT4.

FIG. 30 depicts cell killing curves for EL4 mouse lymphoma cells treated with proaerolysin or the modified proaerolysin MPPT4.

FIG. 31 depicts the nucleotide sequence of a modified proaerolysin (MPPT5; SEQ ID NO:40) in accordance with one embodiment of the present invention having a modified activation sequence comprising a uPA cleavage site and a MMP2 cleavage site.

FIG. 32 depicts the amino acid sequence of a modified proaerolysin (MPPT5; SEQ ID NO:41) in accordance with one embodiment of the present invention having a modified activation sequence comprising a uPA cleavage site and a MMP2 cleavage site.

FIG. 33 depicts an SDS-PAGE gel showing cleavage of the modified proaerolysin MPPT5 by MMP2 and uPA.

FIG. 34 depicts cell killing curves for HT 1080 human fibrosarcoma cells treated with proaerolysin or the modified proaerolysin MPPT5.

FIG. 35 depicts cell killing curves for HeLa human cervical cancer cells treated with proaerolysin or the modified proaerolysin MPPT5.

FIG. 36 depicts cell killing curves for A2058 human melanoma cells treated with proaerolysin or the modified proaerolysin MPPT5.

FIG. 37 depicts cell killing curves for EL4 mouse lymphoma cells treated with proaerolysin or the modified proaerolysin MPPT5.

FIG. 38 depicts the nucleotide sequence of a mutant proaerolysin (PA-R336A; SEQ ID NO:42), which comprises an arginine to alanine substitution at amino acid 336 in the large binding domain of proaerolysin.

FIG. 39 depicts the amino acid sequence of a mutant proaerolysin (PA-R336A; SEQ ID NO:43), which comprises an arginine to alanine substitution at amino acid 336 in the large binding domain of proaerolysin.

FIG. 40 depicts the ability of the mutant proaerolysin PA-R336A to bind to EL4 mouse lymphoma cells.

FIG. 41 depicts the ability of the mutant proaerolysin PA-R336A to kill EL4 mouse lymphoma cells.

FIG. 42 depicts the nucleotide sequence of a mutant proaerolysin (AFA-PA-R336A; SEQ ID NO:44), which comprises an arginine to alanine substitution at amino acid 336 in the large binding domain of proaerolysin and an AFAI targeting unit.

FIG. 43 depicts the amino acid sequence of a mutant proaerolysin (AFA-PA-R336A; SEQ ID NO:45), which comprises an arginine to alanine substitution at amino acid 336 in the large binding domain of proaerolysin and an AFAI targeting unit.

FIG. 44 depicts the ability of the mutant proaerolysin AFA-PA-R336A to kill (A) EL4 mouse lymphoma cells, and (B) A549 human lung cancer cells.

### DETAILED DESCRIPTION OF THE INVENTION

The present invention provides modified pore-forming protein toxins (MPPTs) capable of killing cancer cells. The MPPTs of the present invention are derived from naturally-occurring pore-forming protein toxins (nPPTs) such as aerolysin and aerolysin-related toxins, and comprise a modified activation sequence that permits activation of the MPPTs in a variety of different cancer types. Thus, in contrast to pore-forming molecules such as those described in International Patent Application No. PCT/US02/27061 (WO 03/018611) which have been engineered to selectively target a specific type of cancer, the MPPTs of the present invention are capable of acting as “broad spectrum” anti-cancer agents. The modified activation sequence of the MPPTs comprises a functional deletion of a native protease cleavage site and addition of one or more cleavage sites, each cleavable by an enzyme which is associated with a plurality of cancer types (a general activating agent) or a plurality of cleavage sites, each of which is cleavable by an enzyme that is associated with a specific type of cancer (a specific activating agent).

The MPPTs according to the present invention can optionally further comprise one or more additional modifications which allow the MPPTs to be “tailored” for selective activation in a specific cancer. These modifications include, but are not limited to, the addition of an artificial regulatory domain capable of either targeting the MPPT to a specific type of cell, and/or inhibiting the activity of the MPPT in such a way that inhibition of the MPPT is released at a target cell, as well as modifications to the cell binding domain(s) of the MPPTs to decrease non-selective binding of the MPPTs.

Thus, the MPPTs of the present invention are designed to act as generally effective anti-cancer cell therapeutics that can be broadly applied to kill numerous types of cancer cells.

Further refinement of these broad spectrum molecules to target them to kill a selected type of cancer cells however, is also encompassed by the present invention. The MPPTs, therefore, are effective in decreasing the growth of a variety of tumors, and may be used in the treatment of various types of cancer.

### DEFINITIONS

Unless defined otherwise, all technical and scientific terms used herein have the same meaning as commonly understood by one of ordinary skill in the art to which this invention belongs.

Generally, the nomenclature used herein and the laboratory procedures in drug discovery, cell culture, molecular genetics, diagnostics, amino acid and nucleic acid chemistry, and sugar chemistry described below are those well known and commonly employed in the art.

Standard techniques are typically used for signal detection, recombinant nucleic acid methods, polynucleotide synthesis, and microbial culture and transformation (e.g., electroporation, lipofection).

The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y.). Standard techniques are used for chemical syntheses, chemical analyses, and biological assays. As employed throughout the disclosure, the following terms, unless otherwise indicated, shall be understood to have the following meanings:

“Binding pair” refers to two moieties (e.g. chemical or biochemical) that have an affinity for one another. Examples of binding pairs include homo-dimers, hetero-dimers, antigen/antibodies, lectin/avidin, target polynucleotide/probe, oligonucleotide, antibody/anti-antibody, receptor/ligand, enzyme/ligand and the like. “One member of a binding pair” refers to one moiety of the pair, such as an antigen or ligand.

“Isolated polynucleotide” refers to a polynucleotide of genomic, cDNA, or synthetic origin or some combination there of, which (1) is not associated with the cell in which the “isolated polynucleotide” is found in nature, or (2) is operably linked to a polynucleotide which it is not linked to in nature.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

“Control sequence” refers to polynucleotide sequences which are necessary to effect the expression of coding and non-coding sequences to which they are ligated. The nature of such control sequences differs depending upon the host organism; in prokaryotes, such control sequences generally include one or more of: promoter, ribosomal binding site, and transcription termination sequence; in eukaryotes, generally, such control sequences include one or more of: promoters and transcription termination sequences. The term “control sequences” is intended to include, at a minimum, components whose presence can influence expression, and can also include additional components whose presence is advantageous, for example, leader sequences and fusion partner sequences.

“Polynucleotide” refers to a polymeric form of nucleotides of at least 10 nucleotides in length, either ribonucleotides or deoxynucleotides or a modified form of either type of nucleotide. The term includes single and double stranded forms of DNA or RNA.

The terms “label” or “labeled” refer to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (or reporter genes) (e.g., horseradish peroxidase, β-galactosidase, β-latamase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by linker arms of various lengths to reduce potential steric hindrance.

The term “gene,” as used herein, refers to a segment of nucleic acid that encodes an individual protein or RNA (also referred to as a “coding sequence” or “coding region”) together with associated regulatory regions such as promoters, operators, terminators and the like, that may be located upstream or downstream of the coding sequence.

The term “selectively hybridize,” as used herein, refers to the ability of a nucleic acid to bind detectably and specifically to a second nucleic acid. Polynucleotides selectively hybridize to target nucleic acid strands under hybridization and wash conditions that minimize appreciable amounts of detectable binding to non-specific nucleic acids. High stringency conditions can be used to achieve selective hybridization conditions as known in the art and discussed herein. Typically, hybridization and washing conditions are performed at high stringency according to conventional hybridization procedures. Washing conditions are typically 1-3×SSC, 0.1-1% SDS, 50-70° C. with a change of wash solution after about 5-30 minutes.

The term “corresponding to” or “corresponds to” indicates that a polynucleotide sequence is identical to all or a portion of a reference polynucleotide sequence. In contradistinction, the term “complementary to” is used herein to indicate that the polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The following terms are used herein to describe the sequence relationships between two or more polynucleotides or two or more polypeptides: “reference sequence,” “window of comparison,” “sequence identity,” “percent sequence identity,” and “substantial identity.” A “reference sequence” is a defined sequence used as a basis for a sequence comparison; a reference sequence may be a subset of a larger sequence, for example, as a segment of a full-length cDNA, gene or protein sequence, or may comprise a complete cDNA, gene or protein sequence. Generally, a reference polynucleotide sequence is at least 20 nucleotides in length, and often at least 50 nucleotides in length. A reference polypeptide sequence is generally at least 7 amino acids in length and often at least 17 amino acids in length.

A “window of comparison”, as used herein, refers to a conceptual segment of the reference sequence of at least 15 contiguous nucleotide positions or at least 5 contiguous amino acid positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide or polypeptide sequences are identical (i.e. on a nucleotide-by-nucleotide or amino acid-by-amino acid basis) over the window of comparison.

The term “percent (%) sequence identity,” as used herein with respect to a reference sequence is defined as the percentage of nucleotide or amino acid residues in a candidate sequence that are identical with the residues in the reference polypeptide sequence over the window of comparison after optimal alignment of the sequences and introducing gaps, if necessary, to achieve the maximum percent sequence identity, without considering any conservative substitutions as part of the sequence identity.

The term “substantial identity” as used herein denotes a characteristic of a polynucleotide or polypeptide sequence, wherein the polynucleotide or polypeptide comprises a sequence that has at least 50% sequence identity as compared to a reference sequence over the window of comparison. Polynucleotide and polypeptide sequences with at least 60% sequence identity, at least 70% sequence identity, at least 80% sequence identity, and at least 90% sequence identity as compared to a reference sequence over the window of comparison are also considered to have substantial identity with the reference sequence.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease or disorder being treated. Thus, the terms therapy and treatment are used in the broadest sense, and include the prevention (prophylaxis), moderation, reduction, and curing of a disease or disorder at various stages. Preventing deterioration of a subject's status is also encompassed by the term. Subjects in need of therapy/treatment thus include those already having the disease or disorder as well as those prone to, or at risk of developing, the disease or disorder and those in whom the disease or disorder is to be prevented.

The term “ameliorate” includes the arrest, prevention, decrease, or improvement in one or more the symptoms, signs, and features of the disease or disorder being treated, both temporary and long-term.

The term “subject” or “patient” as used herein refers to an animal in need of treatment.

The term “animal,” as used herein, refers to both human and non-human animals, including, but not limited to, mammals, birds and fish.

Administration of the compounds of the invention “in combination with” one or more further therapeutic agents, is intended to include simultaneous (concurrent) administration and consecutive administration. Consecutive administration is intended to encompass administration of the therapeutic agent(s) and the compound(s) of the invention to the subject in various orders and via various routes.

The term “inhibit,” as used herein, means to decrease, reduce, slow-down or prevent.

The term “polypeptide” is used herein as a generic term to refer to an amino acid sequence of at least 20 amino acids in length that can be a wild-type (naturally-occurring) protein sequence, a fragment of a wild-type protein sequence, a variant of a wild-type protein sequence, a derivative of a wild-type protein sequence, or an analogue of a wild-type protein sequence. Hence, native protein sequences and fragments, variants, derivatives and analogues of native protein sequences, as defined herein, are considered to be species of the polypeptide genus.

The term “isolated polypeptide,” as used herein, refers to a polypeptide which by virtue of its origin is not associated with other polypeptides with which it is normally associated with in nature, and/or is isolated from the cell in which it normally occurs and/or is free of other polypeptides from the same cellular source and/or is expressed by a cell from a different species, and/or does not occur in nature.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Naturally-occurring,” or “native” as used herein, as applied to an object, refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “activity” as used with respect to a “pore-forming activity” or “activity of modified pore-forming protein toxins” refers to the ability of a naturally occurring pore-forming protein toxin or a modified pore-forming protein toxin to exhibit one or more of: the ability to bind to a cell, the ability to be activated by protease cleavage, the ability to oligomerize, or the ability to insert into or form pores in a cell membrane.

The term “general activating agent” refers to an enzyme, the presence of which is associated with a plurality of different cancer types. By “associated with” is meant that the expression of the enzyme is up-regulated in a cancer cell when compared to a normal cell, or that the enzyme is localized to cancer cells as compared to normal cells, or that the enzyme is produced and/or activated by cells associated with cancerous tissue or cells. In this context, the terms “plurality” and “variety” mean at least two different cancer types. By “different cancer types” is meant cancers originating in different tissues (for example, lung cancer and breast cancer), or in different cells within a tissue (for example lymphoma and leukemia, or large cell, alveolar cell, and small cell lung cancers). In one embodiment, the term “general activating agent” refers to an agent, the presence of which is associated with cells of at least 3 different cancer types. In another embodiment, the term “general activating agent” refers to an agent, the presence of which is associated with cells of at least 4 different cancer types. In a further embodiment, the term “general activating agent” refers to an agent, the presence of which is associated with cells of at least 5 different cancer types. In yet another embodiment, the term “general activating agent” refers to an agent, the presence of which is associated with cells of at least 8 different cancer types.

The term “specific activating agent” refers to an enzyme, the presence of which is associated with a specific type of cancer. By “specific type of cancer” is meant a cancer originating in one tissue (for example breast tissue, lung tissue, or colon tissue), or involving one cell type in a tissue (for example lymphoma, or non-small cell lung cancer cells) or originating in specific cells within a tissue (for example lymphoma and leukemia cells, or large cell, alveolar cell, and small cell lung cancer cells).

The term “mutation,” as used herein, refers to a deletion, insertion, substitution, inversion, or combination thereof, of one or more nucleotides in a polynucleotide sequence when compared to the naturally occurring polynucleotide sequence, or a deletion, insertion, substitution or combination thereof of one or more amino acids in a polypeptide sequence.

The term “cleavage site” as used herein, refers to a sequence of amino acids, nucleotides, sugars or modified versions thereof, which is recognized and selectively cleaved by either a general or specific activating agent.

The term “activation sequence” as used herein, refers to a flexible loop structure in a pore-forming protein toxin that comprises an amino acid sequence that can be cleaved by an appropriate protease, resulting in activation of the pore-forming ability of the protein toxin.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. 1 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

Naturally-occurring amino acids are identified throughout by the conventional three-letter or one-letter abbreviations indicated below, which are as generally accepted in the peptide art and are recommended by the IUPAC-IUB commission in biochemical nomenclature:

TABLE 1
Amino acid codes

| | 3-letter | 1-letter |
|---|---|---|
| Name | code | code |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The peptide sequences set out herein are written according to the generally accepted convention whereby the N-terminal amino acid is on the left and the C-terminal amino acid is on the right. By convention, L-amino acids are represented by upper case letters and D-amino acids by lower case letters.

The term “alkyl,” as used herein, refers to a straight chain or branched hydrocarbon of one to ten carbon atoms or a cyclic hydrocarbon group of three to ten carbon atoms. Said alkyl group is optionally substituted with one or more substituents independently selected from the group of: alkyl, alkenyl, alkynyl, aryl, heteroalkyl, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene and NZ₁Z₂ where Z₁ and Z₂ are independently hydrogen, alkyl, aryl, and aralkyl. This term is exemplified by such groups as methyl, ethyl, n-propyl, i-propyl, n-butyl, t-butyl, 1-butyl (or 2-methylpropyl), cyclopropylmethyl, i-amyl, n-amyl, hexyl, cyclopropyl, cyclobutyl, cyclopentyl, and the like.

The term “alkenyl” refers to a straight chain or branched hydrocarbon of two to ten carbon atoms having at least one carbon to carbon double bond. Said alkenyl group can be optionally substituted with one or more substituents as defined above. Exemplary groups include allyl and vinyl.

The term “alkynyl” refers to a straight chain or branched hydrocarbon of two to ten carbon atoms having at least one carbon to carbon triple bond. Said alkynyl group can be optionally substituted with one or more substituents as defined above. Exemplary groups include ethynyl and propargyl.

The term “heteroalkyl,” as used herein, refers to an alkyl group of 2 to 10 carbon atoms, wherein at least one carbon is replaced with a hetero atom, such as N, O or S.

The term “aryl” (or “Ar”), as used herein, refers to an aromatic carbocyclic group containing about 6 to about 10 carbon atoms or multiple condensed rings in which at least one ring is an aromatic carbocyclic group containing 6 to about 10 carbon atoms. Said aryl or Ar group can be optionally substituted with one or more substituents as defined above. Exemplary aryl groups include phenyl, tolyl, xylyl, biphenyl, naphthyl, 1,2,3,4-tetrahydronaphthyl, anthryl, phenanthryl, 9-fluorenyl, and the like.

The term “aralkyl,” as used herein, refers to a straight or branched chain alkyl, alkenyl or alkynyl group, wherein at least one of the hydrogen atoms is replaced with an aryl group, wherein the aryl group can be optionally substituted with one or more substituents as defined above. Exemplary aralkyl groups include benzyl, 4-phenylbutyl, 3,3-diphenylpropyl and the like.

The term “alkoxy,” as used herein, refers to RO—, wherein R is alkyl, alkenyl or alkynyl in which the alkyl, alkenyl and alkynyl groups are as previously described. Exemplary alkoxy groups include methoxy, ethoxy, n-propoxy, 1-propoxy, n-butoxy, and heptoxy.

The term “aryloxy” as used herein, refers to an “aryl-O—” group in which the aryl group is as previously described. Exemplary aryloxy groups include phenoxy and naphthoxy.

The term “alkylthio,” as used herein, refers to RS—, wherein R is alkyl, alkenyl or alkynyl in which the alkyl, alkenyl and alkynyl groups are as previously described. Exemplary alkylthio groups include methylthio, ethylthio, 1-propylthio and hepthylthio.

The term “arylthio,” as used herein, refers to an “aryl-S-” group in which the aryl group is as previously described. Exemplary arylthio groups include phenylthio and naphthylthio.

The term “aralkyloxy,” as used herein, refers to an “aralkyl-O—” group in which the aralkyl group is as previously described. Exemplary aralkyloxy groups include benzyloxy.

The term “aralkylthio,” as used herein, refers to an “aralkyl-S-” group in which the aralkyl group is as previously described. Exemplary aralkylthio groups include benzylthio.

The term “dialkylamino,” as used herein, refers to an —NZ₁Z₂ group wherein Z₁ and Z₂ are independently selected from alkyl, alkenyl or alkynyl, wherein alkyl, alkenyl and alkynyl are as previously described. Exemplary dialkylamino groups include ethylmethylamino, dimethylamino and diethylamino.

The term “alkoxycarbonyl,” as used herein, refers to R—O—CO—, wherein R is alkyl, alkenyl or alkynyl, wherein alkyl, alkenyl and alkynyl are as previously described. Exemplary alkoxycarbonyl groups include methoxy-carbonyl and ethoxy-carbonyl.

The term “aryloxycarbonyl,” as used herein, refers to an “aryl-O—CO—”, wherein aryl is as defined previously. Exemplary aryloxycarbonyl groups include phenoxy-carbonyl and naphtoxy-carbonyl.

The term “aralkoxycarbonyl,” as used herein, refers to an “aralkyl-O—CO—,” wherein aralkyl is as defined previously. Exemplary aralkoxycarbonyl groups include benzyloxycarbonyl.

The term “acyl” as used herein, refers to RC(O)—, wherein R is alkyl, alkenyl, alkynyl, heteroalkyl, a heterocyclic ring, or a heteroaromatic ring, wherein alkyl, alkenyl, alkynyl, heteroalkyl, heterocyclic, and heteroaromatic are as defined previously.

The term “aroyl” as used herein, refers to an ArC(O)— group, wherein Ar is as defined previously.

The term “carboxy” as used herein, refers to ROC(O)—, wherein R is H, alkyl, alkenyl or alkynyl, and wherein alkyl, alkenyl or alkynyl are as defined previously.

The term “carbamoyl,” as used herein, refers to a H₂N—CO— group.

The term “alkylcarbamoyl,” as used herein, refers to an “Z₁Z₂N—CO—” group wherein one of the Z₁ and Z₂ is hydrogen and the other of Z₁ and Z₂ is independently selected from alkyl, alkenyl or alkynyl and wherein alkyl, alkenyl and alkynyl are as defined previously.

The term “dialkylcarbamoyl,” as used herein, refers to a “Z₁Z₂N—CO—” group wherein Z₁ and Z₂ are independently selected from alkyl, alkenyl or alkynyl and wherein alkyl, alkenyl and alkynyl are as defined previously.

The term “acylamino”, as used herein, refers to an “acyl-NH-” group, wherein acyl is as defined previously.

The term “halo” as used herein, refers to fluoro, chloro, bromo or iodo. In one embodiment, “halo” refers to fluoro, chloro or bromo.

“As used herein, the term “about” refers to a +/−10% variation from the nominal value. It is to be understood that such a variation is always included in any given value provided herein, whether or not it is specifically referred to.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference).

### 1. Modified Pore-Forming Protein Toxins

The modified pore-forming protein toxins (MPPTs) according to the present invention are derived from naturally occurring pore-forming protein toxins (nPPTs) such as aerolysin or aerolysin-related polypeptides. Suitable aerolysin-related nPPTs have the following features: a pore-forming activity that is activated by removal of an inhibitory domain via protease cleavage, and the ability to bind to receptors that are present on cell membranes through one or more binding domains. Examples include, but are not limited to, preproaerolysin and proaerolysin from Aeromonas hydrophila, Aeromonas trota and Aeromonas salmonicida, alpha toxin from Clostridium septicum, anthrax protective antigen, Vibrio cholerae VCC toxin, epsilon toxin from Clostridium perfringens, and Bacillus thuringiensis delta toxins.

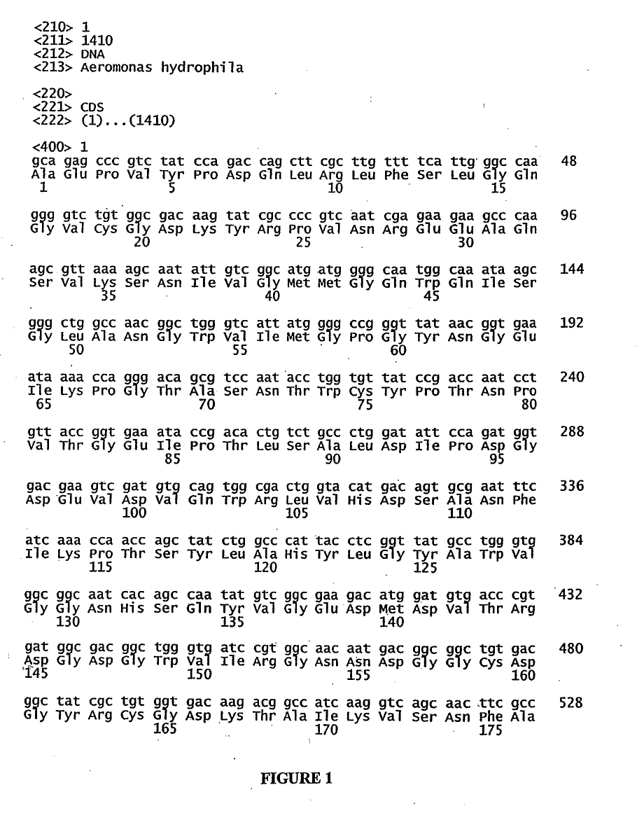

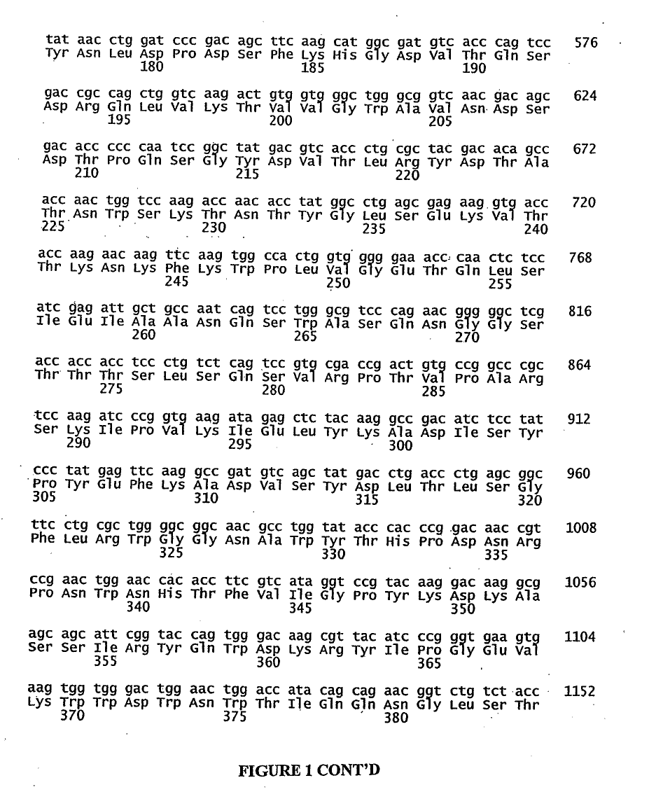

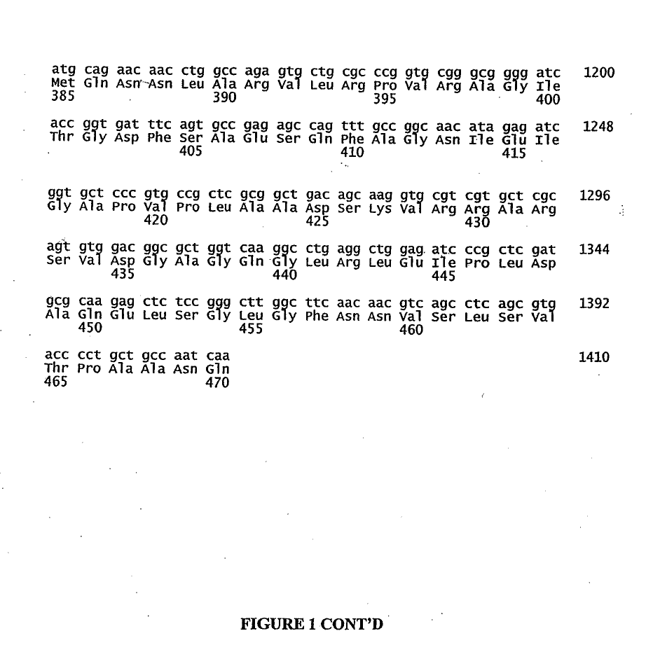

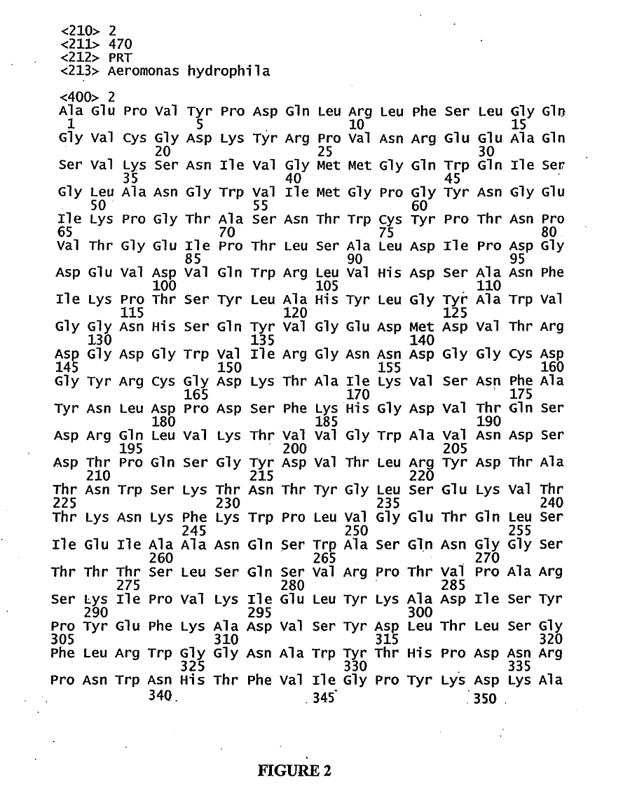

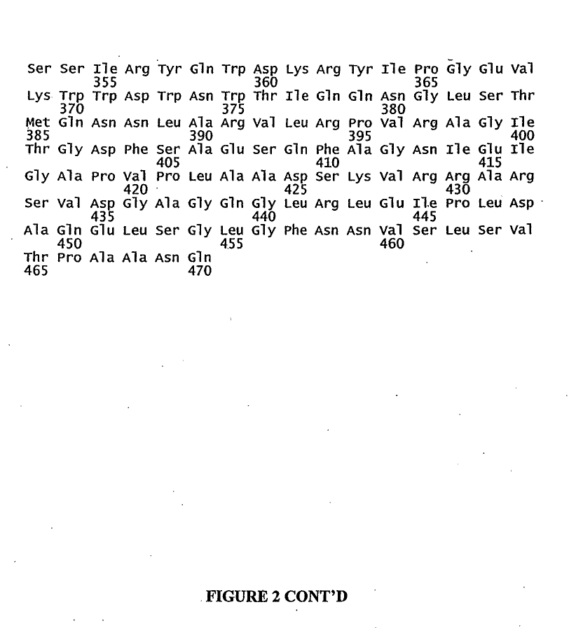

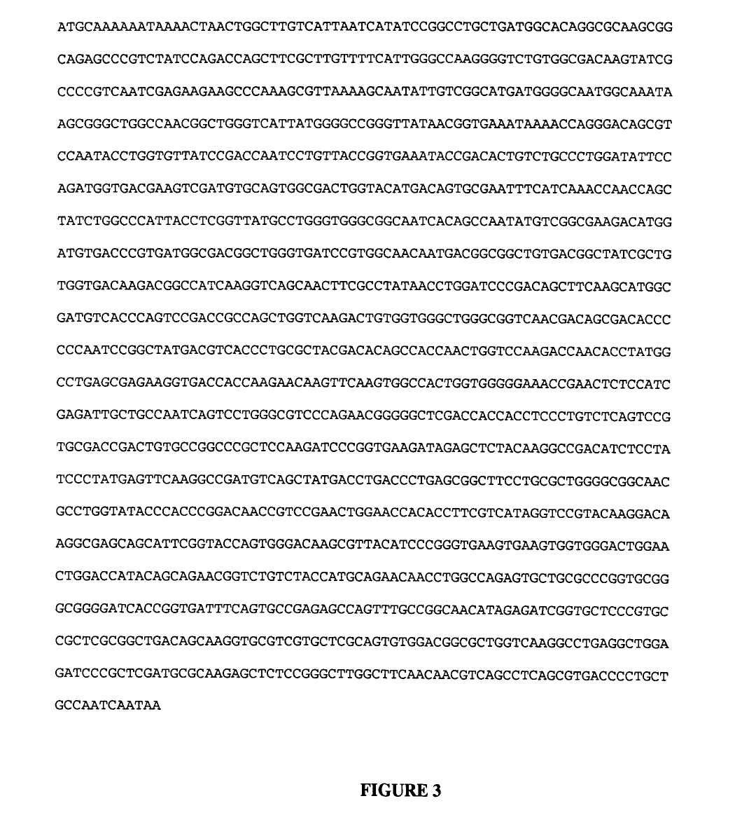

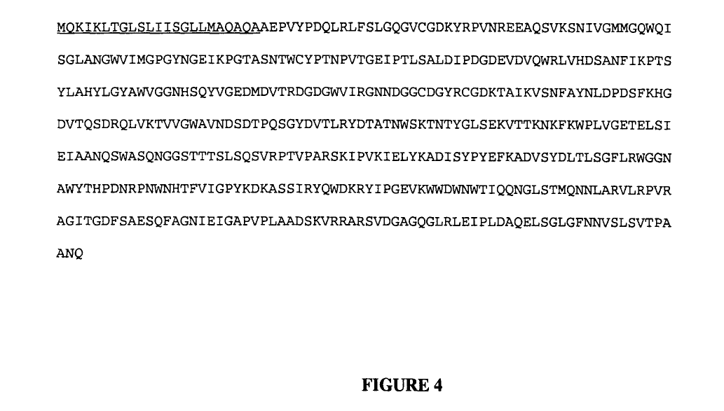

Proaerolysin (PA) polypeptides from the Aeromonas species noted above have been characterized. These polypeptides exhibit greater than 80% pairwise sequence identity between them (Parker et al., Progress in Biophysics & Molecular Biology 88 (2005) 91-142). Each of these PA polypeptides is an approximately 52 kDa protoxin with approximately 470 amino acid residues. A cDNA sequence for wild-type PA from A. hydrophila is shown in SEQ ID NO: 1 (FIG. 1) and the corresponding amino acid sequence of this wild-type PA is shown in SEQ ID NO:2 (FIG. 2). Where applicable, one of skill in the art will understand that MPPTs can be designed based on the sequence of the nPPT with or without a signal sequence. For example, MPPTs can be designed based on the nucleotide sequence for preproaerolysin as shown in FIG. 3 (SEQ ID NO:3) or based on the amino acid sequence for preproaerolysin as shown in FIG. 4 (SEQ ID NO:4). The nucleotide and protein sequences for numerous naturally occurring nPPTs are known in the art and non-limiting examples are listed in the following Table:

TABLE 2
Exemplary nPPTs and corresponding GenBank ™ Accession Numbers

| | Nucleotide | Amino acid |
|---|---|---|
| | sequence | sequence |
| | (GenBank ™ | (GenBank ™ |
| | Accession | Accession |
| nPPT | No.) | No.) |
| Aeromonas hydrophila | Buckley AerA, not | Buckley AerA |
| aerolysin | corrected: M16495 | corrected |
| | | P09167 |
| A. sobria proaerolysin (Husslein, | Y00559 | CAA68642 |
| Chakraborty) | | |
| A. sobria hemolysin (Hirono, I., | X65046 | CAA46182 |
| Aoki, T., Asao, T. and Kozaki, S) | | |
| A. trota proaerolysin (Khan et al) | AF064068 | AAC26217 |
| A. salmonicida hemolysin (Hirono | X65048 | CAA46184 |
| & Aoki) | | |

The A. hydrophila PA protein includes a binding domain (approximately amino acids 1-83 of SEQ ID NO: 2) in what is known as the small lobe of the polypeptide and referred to herein as the small lobe binding domain (SBD), and a C-terminal inhibitory peptide (CIP) domain (approximately amino acids 427-470 of SEQ ID NO: 2) that is removed by protease cleavage at an activation sequence to activate PA. Cleavage at the activation sequence to remove the CIP domain can be carried out by a number of ubiquitous proteases including furin and trypsin. The amino acid residues from approximately 84-426 of SEQ ID NO: 2 are known as the large lobe of the PA polypeptide, and contain a toxin domain and other functional domains, including a second binding domain, referred to herein as the large lobe binding domain (LBD).

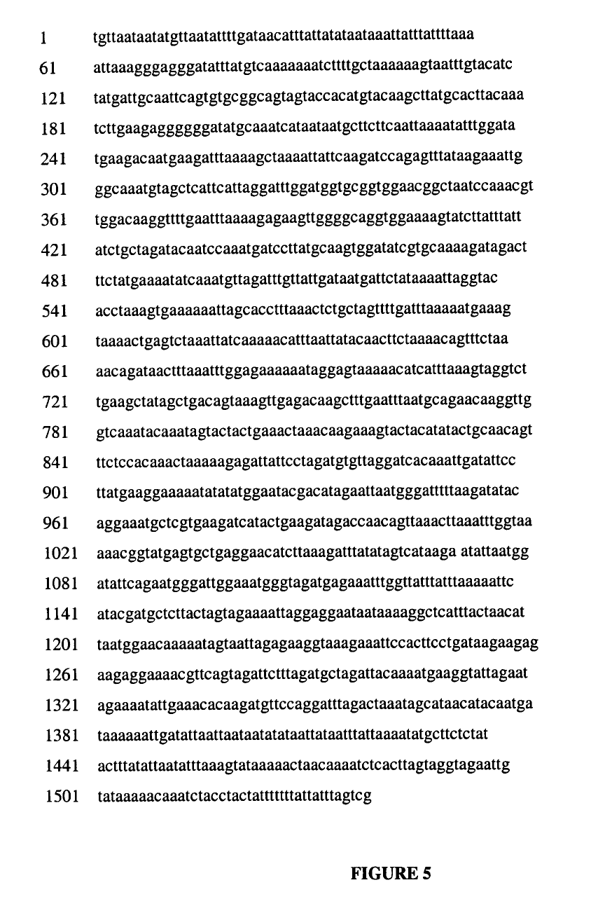

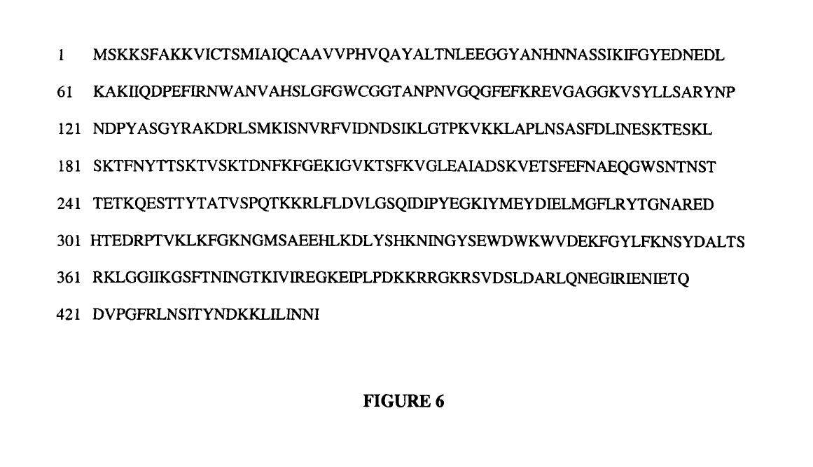

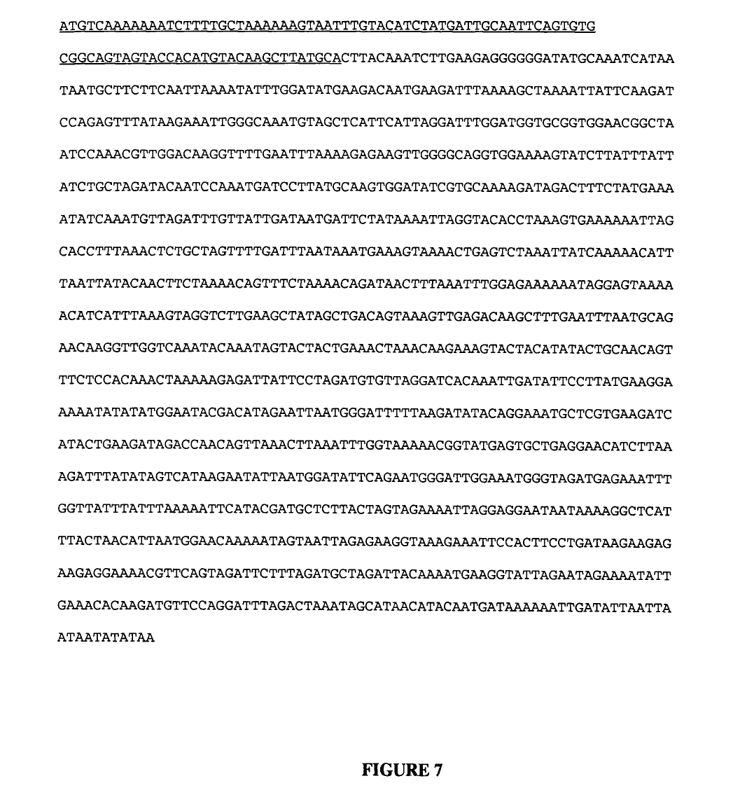

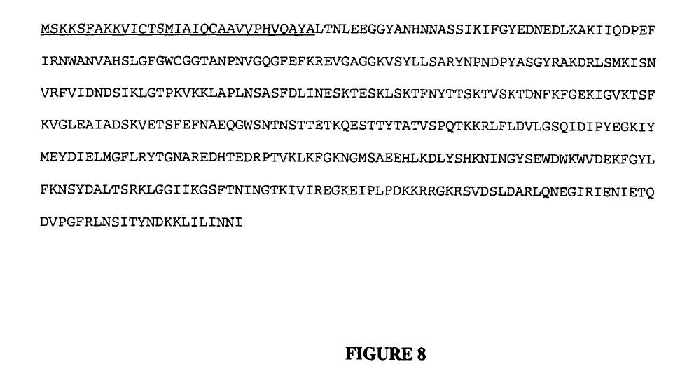

Alpha toxin from C. septicum is considered to be a homologue of proaerolysin based on significant sequence identity and other similarities (Parker et al., supra). Alpha toxin is secreted as a 46,450 Da protoxin (approximately 443 amino acids) that is activated by protease cleavage to remove a C-terminal inhibitory peptide (CIP) domain, and it also binds to glycosyl-phosphatidylinositol (GPI)-anchored proteins. Alpha toxin, however, does not have a region corresponding to the small lobe of PA. An example of a Clostridium septicum alpha toxin nucleic acid sequence is provided in GenBank™ Accession No. S75954. A Clostridium septicum alpha toxin nucleotide sequence is also shown in FIG. 5 (SEQ ID NO:5). An example of a Clostridium septicum alpha toxin protein sequence is provided in GenBank™ Accession No. AAB32892. SEQ ID NO:6, as shown in FIG. 6, is also an example of a Clostridium septicum protein sequence. The sequence of a Clostridium septicum nucleotide sequence including signal sequence (SEQ ID NO:7) is shown in FIG. 7 (signal sequence underlined). An exemplary Clostridium septicum nucleotide sequence including signal sequence (SEQ ID NO:8) is shown in FIG. 8 (signal sequence underlined). Based on sequence homology, alpha toxin is thought to have a similar structure and similar GPI-anchored protein binding activity to that of proaerolysin.

In one embodiment, the MPPTs according to the present invention comprise a modified proaerolysin polypeptide. In a further embodiment, the MPPTs comprise a modified proaerolysin polypeptide from A. hydrophila. In another embodiment of the invention, the MPPTs comprise a modified alpha toxin polypeptide.

The present invention further includes MPPTs that are derived from fragments of nPPTs. Suitable fragments include those that are capable of being activated to form pores in target cells by removal of the CIP domain. i.e. are “functional fragments.” For example, in the case of PA, a suitable fragment would be one that comprised the large lobe of the protein as well as the CIP domain and activation sequence. Thus, in one embodiment of the invention, the MPPT is derived from a fragment of proaerolysin that includes the large lobe, the CIP domain and the activation sequence. In another embodiment, the MPPT is derived from a fragment of proaerolysin that comprises the small lobe, the large lobe, the activation sequence, but only part of a CIP domain. Other functional fragments could be readily determined by the skilled technician using standard techniques known in the art.

MPPTs according to the present invention comprise a modification to the naturally occurring activation sequence that permits activation of the MPPT in a variety of different cancer types. The modification functionally deletes a native protease cleavage site and introduces one or more general cleavage sites, each of which is cleavable by an enzyme, the presence of which is associated with a variety of cancer types, or a plurality of specific cleavage sites, each of which is cleavable by an enzyme, the presence of which is associated with a specific type of cancer. These modifications result in a single MPPT molecule that is capable of being activated to kill numerous types of cancer cells. These modifications are described in greater detail below. In addition, one or more further optional modifications may be made to the MPPTs. These optional modifications allow the MPPT to be selectively activated to kill selected cancer cells. These modifications include one or more modification of the native small lobe binding domain (SBD), one or more modifications of the native large lobe binding domain (LBD), or addition of one or more artificial regulatory domains (ARD). These optional modifications are also described in detail below.

In one embodiment of the invention, the MPPT comprises a modified activation sequence. In another embodiment of the invention, the MPPT comprises a modified activation sequence and one or more modifications to the SBD. In still another embodiment, the MPPT comprises a modified activation sequence and one or more mutations to the LBD. In yet another embodiment, the MPPT comprises a modified activation sequence and one or more ARD. In a further embodiment, the MPPT comprises a modified activation sequence, one or more modifications to the SBD, and one or more modifications to the LBD. In a still further embodiment, the MPPT comprises a modified activation sequence, one or more modifications to the SBD and one or more ARD. In yet a further embodiment, the MPPT comprises a modified activation sequence, one or more modifications to the LBD and one or more ARD. In one more embodiment, the MPPT comprises a modified activation sequence, one or more modifications to the LBD, one or more modifications to the SBD, and one or more ARD.

In one embodiment of the invention, the MPPT comprises a modified activation sequence comprising one or more general cleavage sites. In another embodiment of the invention, the MPPT comprises a modified activation sequence comprising one or more general cleavage sites and one or more modifications to the SBD. In still another embodiment, the MPPT comprises a modified activation sequence comprising one or more general cleavage sites and one or more modifications to the LBD. In yet another embodiment, the MPPT comprises a modified activation sequence comprising one or more general cleavage sites and one or more ARD. In a further embodiment, the MPPT comprises a modified activation sequence comprising one or more general cleavage sites, one or more modifications to the SBD, and one or more modifications to the LBD. In a still further embodiment, the MPPT comprises a modified activation sequence comprising one or more general cleavage sites, one or more modifications to the SBD and one or more ARD. In yet a further embodiment, the MPPT comprises a modified activation sequence comprising one or more general cleavage sites, one or more modifications to the LBD and one or more ARD. In one more embodiment, the MPPT comprises a modified activation sequence comprising one or more general cleavage sites, one or more modifications to the LBD, one or more modifications to the SBD, and one or more ARD.

In one embodiment of the invention, the MPPT comprises a modified activation sequence comprising a plurality of specific cleavage sites. In another embodiment of the invention, the MPPT comprises a modified activation sequence comprising a plurality of specific cleavage sites and one or more modifications to the SBD. In still another embodiment, the MPPT comprises a modified activation sequence comprising a plurality of specific cleavage sites and one or more mutation to the LBD. In yet another embodiment, the MPPT comprises a modified activation sequence comprising a plurality of specific cleavage sites and one or more ARD. In a further embodiment, the MPPT comprises a modified activation sequence comprising a plurality of specific cleavage sites, one or more modifications to the SBD, and one or more modifications to the LBD. In a still further embodiment, the MPPT comprises a modified activation sequence comprising a plurality of specific cleavage sites, one or more modifications to the SBD and one or more ARD. In yet a further embodiment, the MPPT comprises a modified activation sequence comprising a plurality of specific cleavage sites, one or more modifications to the LBD and one or more ARD. In one more embodiment, the MPPT comprises a modified activation sequence comprising a plurality of specific cleavage sites, one or more modifications to the LBD, one or more modifications to the SBD, and one or more ARD.

### 1.1. Modification of Activation Sequence

MPPTs according to the present invention comprise modifications of the naturally occurring activation sequence of the nPPT permitting activation of the MPPTs in a variety of different cancer types. The modified activation sequence comprises one or more general cleavage site modifications, or a plurality of specific cleavage site modifications, resulting in a single MPPT molecule that is capable of being activated to kill numerous types of cancer cells.

### 1.1.1 Modifications Comprising One or More General Cleavage Sites

MPPTs with one or more general cleavage site modifications comprise a modification of the naturally occurring activation sequence to provide one or more cleavage sites for a general activating agent. A general activating agent is an enzyme, the presence of which is associated with a variety of different cancer types. For example, the expression of the enzyme can be up-regulated in a cancer cell compared to a normal cell, or the enzyme can be localized to cancer cells as compared to normal cells, or the enzyme may be produced and/or activated by cancer associated tissue or cells. A general activating agent may be, for example, a protease.

In one embodiment, the MPPT comprises an activation sequence modified to include two or more general cleavage sites, each of the general cleavage sites can be cleaved by the same general activating agent. Alternatively, each of the general cleavage sites can be cleaved by a different activating agent. When more than one general cleavage site is present, these cleavage sites may either be adjacent to each other, may overlap or may be separated by intervening sequences of varying lengths as is known in the art. In another embodiment, the MPPT comprises an activation sequence modified to include one general cleavage site. In still another embodiment, the MPPT comprises an activation sequence modified to include two general cleavage sites. In yet another embodiment, the MPPT comprises an activation sequence modified to include less than five general cleavage sites.

The one or more general cleavage site modifications to the naturally occurring activation sequence may be achieved as is known in the art. This modification results in functional deletion of the naturally occurring activation sequence, or of one or more naturally occurring cleavage sites in the activation sequence. Functional deletion is achieved by mutation, which can result in, for example, partial or complete deletion, insertion, or other variation made to the naturally occurring activation sequence that renders it inactive. In one embodiment, the native activation sequence of the nPPT may be functionally deleted by insertion of one or more general cleavage site. In another embodiment, functional deletion of the naturally occurring activation sequence, or of one or more naturally occurring cleavage sites in the activation sequence is achieved via mutations in one or more amino acid residues of the native activation sequence which result in the creation of one or more general cleavage sites, each of which can be cleaved by a general activating agent. In an alternate embodiment, the native activation sequence of the nPPT is functionally deleted by replacing the naturally occurring activation sequence, or one or more naturally occurring cleavage sites in the activation sequence with one or more general cleavage sites, each of which can be cleaved by a general activating agent.

As described above, the MPPTs according the present invention comprise one or more general cleavage site modifications that provide one or more cleavage sites, each recognized by a general activating agent that is a protease, the presence of which is associated with a variety of different cancer types. In one embodiment of the invention, the general activating agent is a protease that is associated with cancer invasion and metastasis in general. Examples of such proteases include the matrix metalloproteinase (MMP) family, the caspases, elastase, and the plasminogen activator family, as well as fibroblast activation protein. Members of the MMP family include collagenases, stromelysin, gelatinases, and membrane-type metalloproteases. In particular, MMP-2 (gelatinase A), MMP-9 (gelatinase B), and membrane-type 1 MMP (MT1-MMP) have been reported to be most related to invasion and metastasis in various human cancers. Examples of proteases of the plasminogen activator family include uPA (urokinase-type plasminogen activator) and tPA (tissue-type plasminogen activator).

In another embodiment, the protease is up-regulated and/or secreted by cancer cells. Examples of these proteases include some matrix metalloproteases, some cathepsins, tPA, some caspases, and some kallikreins. In still another embodiment, the protease is secreted by cancer-associated cells. Examples of these proteases include matrix metalloproteases, elastase, plasmin, thrombin, and uPA. In a further embodiment, the protease is activated by enzymes expressed by cancer cells. In still another embodiment, the protease is activated by receptors expressed by cancer cells. A non-limiting example of such a protease is uPA, which is activated by the receptor uPAR (urokinase-type plasminogen activator receptor).

As is known in the art, the proteases described above recognize certain cleavage sites. Non-limiting examples of selected cleavage sites recognized by some of these proteases are shown in Table 3. Cleavage sites recognized by other proteases listed above are known in the art.

TABLE 3
Peptide sequences favored by proteases

| | Favoured cleavage sequence “/” | SEQ ID |
|---|---|---|
| Protease | indicates site of cleavage | NO |
| Caspase 1 | YVADI/X | 46 |
| tPA | FGR/X | 47 |
| MMP14 | GGPLG/LYAGG | 48 |
| HK2 | GKAFRR/X | 49 |
| Thrombin | LVPR/GS | 50 |
| uPA | SGR/SAQ | 51 |
| MMP2 | HPVG/LLAR | 52 |

One of skill in the art would understand that cleavage sites other than those listed in Table 3 are recognized by the listed proteases, and can be used as a general protease cleavage site according to the invention.

In another embodiment, the general activating agent is uPA. In another embodiment of the invention the cleavage site added to the activation sequence is a uPA cleavage site. An example of a uPA cleavage site is: SGRSAQ (SEQ ID NO:51). In another embodiment, the general activating agent is a protease that is associated with angiogenesis in general. Examples of such proteases are matrix metalloproteases and caspases.

In one embodiment, the MPPT comprises a general cleavage site that is recognized by uPA, and optionally one or more other general cleavage sites. In another embodiment, the MPPT comprises a general cleavage site that is recognized by a MMP.

### 1.1.2 Modifications Comprising a Plurality of Specific Cleavage Sites

MPPTs with a plurality of specific cleavage site modifications comprise modification of the naturally occurring activation sequence to include two or more different types of specific cleavage sites, each type capable of being cleaved by a specific activating agent. Each specific cleavage site is recognized by a different specific activating agent. The two or more different types of cleavage sites may further comprise a cleavage site for a general activating agent. A specific activating agent is an enzyme, the presence of which is associated with a specific type of cancer. For example, expression of the enzyme can be up-regulated in a specific type of cancer cell, or the enzyme can be localized to a specific type of cancer cell, or the enzyme may be produced by a cell that is associated with a specific type of cancer. A specific activating agent may be, for example, a protease.

Modifications comprising a plurality of specific cleavage sites may be achieved as is known in the art, and described above. This modification also results in functional deletion of the naturally occurring activation sequence, or of one or more naturally occurring cleavage sites in the activation sequence. In one embodiment, the native activation sequence of the nPPT is functionally deleted by insertion of a plurality of specific cleavage sites. In another embodiment, functional deletion of the naturally occurring activation sequence is achieved via mutations in the amino acid sequence of the naturally occurring activation sequence, resulting in the addition of two or more specific cleavage sites, each of which can be cleaved by a specific activating agent. In an alternate embodiment, the native activation sequence of the nPPT may be replaced with two or more specific cleavage sites, each of which is capable of being cleaved by a specific activating agent. As is known in the art, the specific cleavage sites may either be adjacent to each other, may overlap or may be separated by intervening sequences of varying lengths as is known in the art.

In another embodiment of the invention, the plurality of specific cleavage site modifications adds two or more cleavage sites, each of which is recognized by a specific activating agent that is a protease. In another embodiment of the invention, the specific activating agent is a protease that is associated with invasion and metastasis of a specific cancer. In a further embodiment of the invention, the specific activating agent is a protease, the expression of which is up-regulated in a specific cancer. In still another embodiment, the specific activating agent is a protease that is produced by a cell that is associated with a specific cancer.

In another embodiment, the specific activating agent is a protease that is associated with lung cancer.

### 1.2. Modifications to Binding Domain(s)

MPPTs according to the present invention are derived from nPPTs that comprise one or more binding domains, as known in the art. In the context of the present invention, when an nPPT comprises one binding domain, it is considered to be a “large lobe binding domain.” MPPTs according to the present invention may comprise modifications to one or more binding domains, as applicable. For example, native proaerolysin from Aeromonas species comprises two binding domains, a small lobe binding domain, and a large lobe binding domain. In contrast, native alpha toxin from Clostridium septicum comprises only a large lobe binding domain. In one embodiment, modifications of the binding domains include functional deletion of a binding domain. A functionally deleted binding domain in an MPPT results in an MPPT that has an attenuated ability to bind to its cell surface receptors, yet still retains pore-forming ability. Functional deletions can be made by deleting or mutating one or more binding domain of an MPPT. In one embodiment, the entire binding domain or portions thereof, may be deleted. In an additional embodiment, insertion of heterologous sequences into the binding domain may also be used to functionally delete the binding domain. Addition of these heterologous sequences may confer an additional functionality to the MPPT. For example, addition of a heterologous sequence can result in the addition of a region that can function as an ARD as described in Section 1.3 below. In an alternative embodiment, an MPPT may comprise a blocking group that functions to prevent interaction of the binding domain with its cell membrane receptor. Methods of attaching blocking groups to MPPTs are known in the art and described in Section 2 below. In still another embodiment, point mutations to the amino acid sequence of the native binding domain of the nPPT can also be made to decrease the ability of the binding domain to bind to its receptor. Further details regarding these modifications are described below.

MPPTs with functional deletions in the binding domain may be carried out using methods known in the art. These methods include the use of recombinant DNA technology as described in Sambrook et al., supra. Alternatively, functional deletions of the binding domain may also be achieved by direct modification of the protein itself according to methods known in the art, such as proteolysis to generate fragments of the MPPT, which can then be chemically linked together (See Section 2.2).

In one embodiment of the invention, the MPPT is modified by functional deletion of its small lobe binding domain (SBD). Exemplary functional deletions of the SBD may be made in the A. hydrophila proaerolysin polypeptide as follows. The entire SBD, corresponding to amino acid 1-83 of SEQ ID NO:2 may be deleted, or portions of this region may be deleted, for example amino acids 45-66 of SEQ ID NO:2. Alternatively, one or more point mutations can be made at the following positions: W45, 147, M57, Y61, K₆₆ (amino acid numbers refer to SEQ ID NO: 2). Exemplary mutations include, but are not limited to W45A, 147E, M57A, Y61A, Y61C, K66Q (amino acid numbers refer to SEQ ID NO: 2) and as described in Mackenzie et al. J. Biol. Chem. 274: 22604-22609, 1999.

In one embodiment of the invention, the MPPT is modified by functional deletion of its large lobe binding domain (LBD). Exemplary functional deletions of the LBD of MPPTs, based on proaerolysin (contained in approximately amino acid residues 84-426 of SEQ ID NO:2) may be made as follows. The entire LBD of proaerolysin may be deleted. Alternatively, in one embodiment of the invention, the MPPT based on proaerolysin comprises one or more point mutations in the LBD to amino acid residues Y162, W324, R323, R336, and/or W127. In another embodiment of the invention, the MPPT based on proaerolysin comprises one or more point mutations in W127 and/or R336. In still another embodiment, the MPPT based on proaerolysin comprises the point mutations Y162A and/or W324A. In a further embodiment the MPPT based on proaerolysin comprises the point mutations R336A, R336c, and/or W127T. In another embodiment, MPPTs comprise mutations to other residues that interact directly with the GPI-protein ligand.

Exemplary mutations to the LBD of MPPTs derived from alpha toxin are noted below and include at least one substituted amino acid in the receptor binding domains of the alpha toxin which include amino acid residues 53, 54, 62, 84-102, 259-274 and 309-315 of the sequence of the native alpha toxin as shown in SEQ ID NO: 6. In one embodiment of the invention, MPPTs derived from alpha toxin include mutations to one or more of the following residues: W85, Y128, R292, Y293, and R305.

### 1.3. Addition of Artificial Regulatory Domain (ARD)

The MPPTs according to the present invention may be optionally modified by the addition of one or more artificial regulatory domains (ARDs). ARDs that can be added to MPPTs include targeting units that are capable of selectively targeting the MPPT to one or more target cell (for example one or more organs, tissues or cell types), and inhibitory units that are capable of inhibiting the activity of the MPPT. One skilled in the art will recognize the possibility that an ARD can function as both a targeting unit and an inhibitory unit. According to one embodiment of the invention, an MPPT is modified by the addition of one or more ARD that is a targeting unit. In another embodiment of the invention, the MPPT is modified by the addition of one or more ARD that is an inhibitory unit. In still another embodiment the MPPT is modified by the addition of an ARD that can function as both a targeting unit and an inhibitory unit. In an additional embodiment, the ARD is capable of functioning to inhibit binding to normal cells, yet able to direct binding to cancer cells. In a further embodiment, the addition of an ARD can function to both target the MPPT to cancer cells, and inhibit the ability of the MPPT to bind to cell surface receptors that are recognized by the nPPT it is derived from. ARDs according to the present invention may be proteins, peptides, or other moieties.

### 1.3.1 Targeting Units

The targeting units according to the present invention may be used to provide target selectivity to the MPPT. For example, the targeting unit can direct the MPPT to the target cell, where the MPPT can be activated and subsequently kill the target cell. Additionally, the targeting unit may act as an inactivator of the MPPT. In this regard, the targeting unit may charge neutralize or sterically inhibit the MPPT from pore formation. The targeting unit may be added to the N- or C-terminus, or both. Alternatively, the targeting unit may be added to any region of the MPPT, including the SBD or the LBD, as long as it does not interfere with the pore-forming activity of the MPPT.

The targeting unit can be a ligand that binds selectively to the target cell. In one embodiment, the targeting unit is an antibody. Antibodies contemplated by the instant invention can be a full-length (i.e., naturally occurring or formed by normal immunoglobulin gene fragment recombinatorial processes) immunoglobulin molecule (for example, an IgG antibody) or an immunologically active (i.e., specifically binding) portion of an immunoglobulin molecule, and may or may not be humanized. Suitable antibodies can be polyclonal or monoclonal antibodies. Alternatively, the antibody may be a single chain antibody as known in the art and described in Bird et al. Science 242:423-6, 1988; and Huston et al., Proc. Natl. Acad. Sci. 85:5879-83, 1988. In one embodiment, an antibody includes camelized antibodies (for example see Tanha et al., J. Biol. Chem. 276:24774-80, 2001).

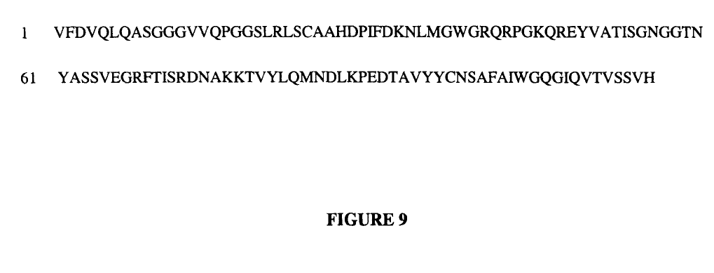

In one embodiment the targeting unit is an antibody that selectively binds a specific type of cancer cell. In another embodiment, the targeting unit is an antibody that selectively binds a lung cancer cell. In yet another embodiment, the targeting unit is an antibody that selectively binds non-small cell lung cancer cells. For example, the targeting unit may be an AFAI antibody or fragment thereof as shown in FIG. 9 (SEQ ID NO:9) that is capable of selectively binding lung cancer cells. AFAI is a single domain antibody that binds cell adhesion molecule 6. The pentavalent form of the antibody is known as ESI (see Mai, K. T., et al, 2006, Histopathyology, 49:515-522) and is also suitable for use as a targeting unit in accordance with the present invention.

In another embodiment, the targeting unit is an antibody that recognizes one of: carcino embryonic antigen CEA (for example, catalog number AB 10036 from Abcam Inc., Cambridge, Mass., recognizes colorectal cancer cells), CA-15-3 (for example, catalog number RDI-CA153-AG from Research Diagnostics and recognizes breast cancer cells), thyroid transcription factor 1 (TTF1, for example, catalog number AB 869 from Abcam Inc., Cambridge, Mass., recognizes lung and thyroid carcinomas), and cytokeratin 7 (for example, catalog number RDI-PR061025 from Research Diagnostics Inc., Flanders, N.J., recognizes epithelial cells of ovary, lung and breast). In another embodiment, the targeting unit is an antibody that recognizes p97 melanotransferrin antigen in melanoma cells, or the L6 antigen on renal cell carcinomas.

In another embodiment, the targeting unit is the antibody Rituxan, which targets CD20 and B-cell non-Hodgkin's lymphoma cells. Alternatively, the targeting unit is Herceptin®. (Genentech) which recognizes some breast cancers and lymphomas; Alemtuzumab (MabCampath®) which binds to CD52, a molecule found on white blood cells and recognizes chronic lymphocytic leukemia cells; Lym-1 (Oncolym®, Schering), which binds to the HLA-DR-encoded histocompatibility antigen that can be expressed at high levels on lymphoma cells; Bevacizumab (Avastin®, Avastin), which binds to vascular endothelial growth factor (VEGF) thus blocking its action and depriving the tumor of its blood supply, or Cetuximab (Erbitux®, Merck) which binds to colorectal cancers.

In another embodiment, the targeting unit is a molecule or ligand that recognizes or is capable of specific binding to a second molecule that is selectively expressed on the target cell. In one embodiment, the targeting unit is a ligand that is specific for a receptor that is selectively expressed on the target cell. Examples of such ligands are: hormones such as steroid hormones, or peptide hormones; neuroactive substances, for example opioid peptides; insulin; growth factors, e.g., epidermal growth factor, insulin-like growth factor, fibroblast growth factor, platelet derived growth factor, tumor necrosis factor; cytokines, for example, an interleukin (IL), e.g., IL-2, IL-4, or IL-5; melanocyte stimulating hormone; a substance or receptor which has affinity for a particular class of cells (or viruses) for example, cancer cells, virally infected cells, immune cells, for example, B cells or T cells or a subset thereof, for example, soluble fragments of CD4, which bind to the protein gp120 expressed on HIV-infected cells; or a substance with an affinity for a class of molecules, for example, a lectin, such as concanavalin A, which binds a subset of glycoproteins. Adhesion molecules, for example, molecules expressed on cells of hematopoetic origin, such as CD2, CD4, CD8 which are expressed on T cells, selectins, integrins, as well as adhesion molecules expressed on non-immune cells, may also be used as targeting units to direct the MPPT of the invention to target cells. Since some cancer cells abnormally express certain adhesion molecules, receptors or recognition motifs for such adhesion molecules may also be used as targeting units. For example, RGD motifs, which function as integrin binding motifs, can be used as targeting units.

In one embodiment, the targeting unit selectively directs the MPPT to cancer cells. In another embodiment the targeting unit selectively directs the MPPT to cells that are actively proliferating. In another embodiment the targeting unit selectively directs the MPPT to a specific tissue or organ. In still another embodiment, the targeting unit selectively directs the MPPT to a lung cancer cell. In a further embodiment, the targeting unit selectively directs the MPPT to cells required for cancer growth, for example, cells forming vasculature for a tumor.

In yet another embodiment, the targeting unit is additionally capable of acting as an inhibitory unit. For example, the AFAI antibody fragment described above is capable of reducing the activity of the MPPT, as well as selectively targeting the MPPT to a lung cancer cell. Similarly, other proteins of similar size are capable of acting as an inhibitory unit.

### 1.3.2 Inhibitory Units

As noted above, the ARD may be an inhibitory unit. Without being limited by mechanism, an inhibitory unit may inactivate the MPPT by, for example, charge neutralization, and/or by sterically inhibiting either the ability of the MPPT to bind to its receptor on the cell membrane, or the ability of the MPPT to form pores in the cell membrane. Examples of suitable inhibitory units include, but are not limited to, antibodies, antibody fragments, enzymes, carbohydrates, peptides, ubiquitin, a phage (via phage engineering) or a streptavidin microbead via peptide biotinylation.

In one embodiment of the invention, the inhibitory unit is an antibody. In a further embodiment, the inhibitory unit is an AFAI antibody fragment. In still another embodiment the inhibitory unit is also capable of functioning as a targeting unit.

In another embodiment, the inhibitory unit is an enzyme. In a further embodiment, the inhibitory unit is a lipase.

### 1.3.3 Addition of ARDs

The ARDs according to the present invention may be added to any region of the MPPT, including, but not limited to, the N- or C-terminus of the MPPT, provided that, for those ARDs that are not cleaved from the MPPT after fulfilling their targeting unit function, the ARD does not interfere with the ability of the MPPT to form pores. Moreover, the ARD may replace or functionally delete a functional domain of the nPPT, such as, for example, a binding domain. The ARD may be directly added to the MPPT, or it may be added via an appropriate linker. Methods of adding additional domains, such as ARDs, to proteins are known in the art. Such methods include covalent linkage of the domain to the MPPT. For example, ARDs may be added to the MPPT via covalent crosslinking (see Woo et al., Arch. Pharm. Res. 22(5):459-63, 1999 and Debinski and Pastan, Clin. Cancer Res. 1(9):1015-22, 1995). Crosslinking can be non-specific, for example by using a homobifunctional-lysine-reactive crosslinking agent, or it can be specific, for example by using a crosslinking agent that reacts with amino groups on the ARD and with cysteine residue located in the MPPT. For example, in the proaerolysin polypeptide, amino acids Cys19, Cys75, Cys159, and/or Cys164 as noted in SEQ ID NO: 2 may be used to crosslink an ARD to the proaerolysin polypeptide. Many other cross-linking agents and linkers are known in the art and are suitable for use in the present invention and include those described in Section 1.3.4.

If the ARD is a protein, recombinant DNA technology can be used to add the ARD to produce the MPPT. Details of suitable recombinant DNA technology can be found, for example, in Sambrook et al. (ed.), Molecular Cloning: A Laboratory Manual 2nd ed., vol. 1-3, Cold Spring Harbor Laboratory Press, Cold Spring, Harbor, N.Y., 1989.

In one embodiment, the ARD is added to the N-terminus of the MPPT. In another embodiment, the ARD is added to the N-terminus of a MPPT derived from proaerolysin. In still another embodiment, the ARD is added to the N-terminus of a MPPT derived from alpha toxin. In a further embodiment the ARD is added to the C-terminus of the MPPT. In another embodiment, the ARD is added to the C-terminus of a MPPT derived from proaerolysin. In yet another embodiment, the ARD is added to the C-terminus of a MPPT derived from alpha toxin. In still another embodiment, the ARD is added to a MPPT derived from proaerolysin using recombinant DNA methods, such as those described in Section 2.

### 1.3.4. Addition of ARD Via a Linker

According to the present invention, ARDs may be covalently attached to MPPTs through an appropriate linker or spacer. In the context of the present invention, the linker acts as a molecular bridge to link the ARD entity to the MPPT entity. The linker can serve, for example, simply as a convenient way to link the two entities, as a means to spatially separate the two entities, to provide an additional functionality to the MPPT, or a combination thereof. For example, it may be desirable to spatially separate the ARD and the MPPT to prevent the ARD from interfering with the activity of the MPPT and/or vice versa. The linker can also be used to provide, for example, lability to the connection between the ARD and the MPPT, an enzyme cleavage site (for example a cleavage site for a protease), a stability sequence, a molecular tag, a detectable label, or various combinations thereof.

The selected linker can be bifunctional or polyfunctional, i.e. contains at least a first reactive functionality at, or proximal to, a first end of the linker that is capable of bonding to, or being modified to bond to, the ARD and a second reactive functionality at, or proximal to, the opposite end of the linker that is capable of bonding to, or being modified to bond to, the MPPT. The two or more reactive functionalities can be the same (i.e. the linker is homobifunctional) or they can be different (i.e. the linker is heterobifunctional). A variety of bifunctional or polyfunctional cross-linking agents are known in the art that are suitable for use as linkers (for example, those commercially available from Pierce Chemical Co., Rockford, Ill.). Alternatively, these reagents can be used to add the linker to the ARD and/or MPPT.

The length and composition of the linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, the linker should not significantly interfere with the regulatory ability of the ARD relating to targeting or inhibition of the MPPT, or with the activity of the MPPT relating to activation, or pore-forming ability.

Linkers suitable for use according to the present invention may be branched, unbranched, saturated, or unsaturated hydrocarbon chains, including peptides as noted above. Furthermore, if the linker is a peptide, the linker can be attached to the MPPT and/or the ARD (if the ARD is also a peptide or protein) using recombinant DNA technology. Such methods are well-known in the art and details of this technology can be found, for example, in Sambrook et al., supra.

In one embodiment of the present invention, the linker is a branched or unbranched, saturated or unsaturated, hydrocarbon chain having from 1 to 100 carbon atoms, wherein one or more of the carbon atoms is optionally replaced by —O— or —NR— (wherein R is H, or C1 to C6 alkyl), and wherein the chain is optionally substituted on carbon with one or more substituents selected from the group of (C1-C6)alkoxy, (C3-C6)cycloalkyl, (C1-C6)alkanoyl, (C1-C6)alkanoyloxy, C1-C6)alkoxycarbonyl, (C1-C6)alkylthio, amide, azido, cyano, nitro, halo, hydroxy, oxo (═O), carboxy, aryl, aryloxy, heteroaryl, and heteroaryloxy.

Examples of suitable linkers include, but are not limited to, peptides having a chain length of 1 to 100 atoms, and linkers derived from groups such as ethanolamine, ethylene glycol, polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains.

In one embodiment, the linker is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6)alkoxy, (C1-C6)alkanoyl, (C1-C6)alkanoyloxy, (C1-C6)alkoxycarbonyl, (C1-C6)alkylthio, amide, hydroxy, oxo (═O), carboxy, aryl and aryloxy.

In another embodiment, the linker is an unbranched, saturated hydrocarbon chain having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6)alkoxy, (C1-C6)alkanoyl, (C1-C6)alkanoyloxy, (C1-C6)alkoxycarbonyl, (C1-C6)alkylthio, amide, hydroxy, oxo (═O), carboxy, aryl and aryloxy.

In a specific embodiment of the present invention, the linker is a peptide having a chain length of 1 to 50 atoms. In another embodiment, the linker is a peptide having a chain length of 1 to 40 atoms.

Peptide linkers which are susceptible to cleavage by enzymes of the complement system, urokinase, tissue plasminogen activator, trypsin, plasmin, or another enzyme having proteolytic activity may be used in one embodiment of the present invention. According to another embodiment of the present invention, an MPPT is attached via a linker susceptible to cleavage by enzymes having a proteolytic activity such as a urokinase, a tissue plasminogen activator, plasmin, thrombin or trypsin. In addition, MPPTs may be attached via disulfide bonds (for example, the disulfide bonds on a cystine molecule) to the ARD molecule. Since many tumors naturally release high levels of glutathione (a reducing agent) this can reduce the disulfide bonds with subsequent release of the MPPT at the site of delivery.

In one embodiment, the ARD is linked to an MPPT by a cleavable linker region. In another embodiment of the invention, the cleavable linker region is a protease-cleavable linker, although other linkers, cleavable for example by small molecules, may be used. Examples of protease cleavage sites are those cleaved by factor Xa, thrombin and collagenase. In one embodiment of the invention, the protease cleavage site is one that is cleaved by a protease that is up-regulated or associated with cancers in general. Examples of such proteases are uPA, the matrix metalloproteinase (MMP) family, the caspases, elastase, and the plasminogen activator family, as well as fibroblast activation protein. In still another embodiment, the cleavage site is cleaved by a protease secreted by cancer-associated cells. Examples of these proteases include matrix metalloproteases, elastase, plasmin, thrombin, and uPA. In another embodiment, the protease cleavage site is one that is up-regulated or associated with a specific cancer. Various cleavage sites recognised by proteases are known in the art and the skilled person will have no difficulty in selecting a suitable cleavage site. Non-limiting examples of cleavage sites are provided in Table 3. Other examples include, but are not limited to, a protease cleavage site targeted by Factor Xa: IEGR (SEQ ID NO:57); a protease cleavage site targeted by Enterokinase is DDDDK (SEQ ID NO:58); and a protease cleavage site targeted by Thrombin is LVPRG (SEQ ID NO:59). As is known in the art, other protease cleavage sites recognized by these proteases can also be used. In one embodiment, the cleavable linker region is one which is targeted by endocellular proteases.

As known in the art, the attachment of a linker to a MPPT (or of a linker element to an ARD, or an ARD to a MPPT) need not be a particular mode of attachment or reaction. Various reactions providing a product of suitable stability and biological compatibility is acceptable.

### 1.4. Other Modifications

The present invention contemplates further modification of MPPTs that do not affect the ability of the MPPTs to selectively target cancer cells. Such modifications include amino acid substitutions, insertions or deletions, and modifications, for example, to reduce antigenicity of the MPPT, to enhance the stability of the MPPT and/or to improve the pharmacokinetics of the MPPTs. In one embodiment, further modifications to MPPTs result in a polypeptide that differs by only a small number of amino acids from the MPPT. Such modifications include deletions (for example of 1-3 or more amino acids), insertions (for example of 1-3 or more residues), or substitutions that do not interfere with the ability of the MPPTs to selectively target and kill cancer cells. In one embodiment, further modifications to the MPPTs result in a polypeptide that retains at least 70%, 80%, 85%, 90%, 95%, 98%, or greater sequence identity to the MPPT and maintains the ability of the MPPT to selectively target and kill cancer cells.

MPPTs may be modified by substitution whereby at least one residue in the amino acid sequence has been removed and a different residue inserted in its place. In one embodiment, the substitution is a conservative substitution. A conservative substitution is one in which one or more amino acids (for example 2, 5 or 10 residues) are substituted with amino acid residues having similar biochemical properties. Typically, conservative substitutions have little to no impact on the activity of a resulting polypeptide. For example, ideally, an MPPT including one or more conservative substitutions retains the activity of the corresponding nPPT. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gin for Lys; Leu or Ile for Met; Met, Leu or Tyr for Phe; Thr for Ser; Ser for Thr; Tyr for Trp; Trp or Phe for Tyr; and Ile or Leu for Val.

In another embodiment the substitution is a permissive substitution. Permissive substitutions are non-conservative amino acid substitutions, but also do not significantly alter MPP activity. An example is substitution of Cys for Ala at position 300 of SEQ ID NO: 2 in a proaerolysin polypeptide. Other non-conservative substitutions that do not affect the activity of the MPPT can be readily determined by the skilled technician.

An MPPT can be modified to include one or more substitutions by manipulating the nucleotide sequence that encodes that polypeptide using, for example, standard procedures such as site-directed mutagenesis or PCR. Further information about substitutions can be found in, among other locations, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

In one embodiment, MPPTs are modified to include 1 or more amino acid substitutions of single residues. In another embodiment, the MPPTs are modified to include 1 amino acid substitution. In another embodiment, the MPPTs are modified to include from about 2 to about 10 amino acid substitutions. In another embodiment, the MPPTs are modified to include about 3 to about 5 amino acid substitutions.

Non-limiting examples of further modifications that may be made to MPPTs derived from proaerolysin in various embodiments of the invention include substitutions at one or more of positions 22, 107, 114, 121, 127, 135, 159, 164, 171, 186, 198, 201, 202, 203, 216, 220, 238, 248, 249, 250, 252, 253, 254, 256, 258, 259, 263, 284, 285, 293, 294, 296, 299, 300, 309, 332, 341, 349, 361, 369, 371, 372, 373, 416, 417, 418, 445 and 449. Specific non-limiting examples are listed in Table 4.

TABLE 4
Exemplary single mutations of MPPs derived from a native proaerolysin
polypeptide

| | | | | |
|---|---|---|---|---|
| H107N | G202C | G251C | T284C | H341N |
| K22C | H121N | W203C | E252C | V285C |
| W127T | T253S | V293C | K361C | N459C |
| C164S | D216C | T253C | K294C | K369Q |
| Q254C | K294Q | W371L | D372N | I445C |
| Y135A | R220Q | E296C | K299C | K349C |
| Y135F | K171C | K238C | W373L | A418C |
| K22C | A300C | S256C | K309C | H332N |
| H186N | P248C | E258C | I416C | Q263C |
| K198C | L249C | I259C | G417C | |
| K114C | C159S | V201C | V250C | |

Peptidomimetic and organomimetic embodiments are also contemplated, whereby the three-dimensional arrangement of the chemical constituents of such peptido- and organomimetics mimic the three-dimensional arrangement of the polypeptide backbone and component amino acid side chains in the polypeptide, resulting in such peptido- and organomimetics of an MPPT which have the ability to lyse cancer cells. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in CADD.

Other modifications that may be made to the MPPTs include, for example, modifications to the carboxylic acid groups of the MPPT, whether carboxyl-terminal or side chain, in which these groups are in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the polypeptide, whether amino-terminal or side chain, can be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Other modifications include conversion of hydroxyl groups of the polypeptide side chain to C₁-C₁₆ alkoxy or to a C₁-C₁₆ ester using well-recognized techniques. Phenyl and phenolic rings of the polypeptide side chain can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the polypeptide side chains can be extended to homologous C₂-C₄ alkylenes. Thiols can be protected with any one of a number of well-recognized protecting groups, such as acetamide groups. Those skilled in the art will also recognize methods for introducing cyclic structures into the polypeptides described herein to select and provide conformational constraints to the structure that result in enhanced stability. For example, a carboxyl-terminal or amino-terminal cysteine residue can be added to the polypeptide, so that when oxidized the polypeptide will contain a disulfide bond, generating a cyclic peptide. Other peptide cyclizing methods include the formation of thioethers and carboxyl- and amino-terminal amides and esters.

The present invention further contemplates that the MPPT can comprise further modifications intended to improve the pharmacokinetic properties of the molecule when administered to a subject. Various modifications to reduce immunogenicity and/or improve the half-life of therapeutic proteins are known in the art. For example, the MMPTs can undergo glycosylation, isomerization, or deglycosylation according to standard methods known in the art. Similarly, the MPPT can be modified by non-naturally occurring covalent modification for example by addition of polyethylene glycol moieties (pegylation) or lipidation. In one embodiment, the MPPTs of the invention are conjugated to polyethylene glycol (PEGylated) to improve their pharmacokinetic profiles. Conjugation can be carried out by techniques known to those skilled in the art (see, for example, Deckert et al., Int. J. Cancer 87: 382-390, 2000; Knight et al., Platelets 15: 409-418, 2004; Leong et al., Cytokine 16: 106-119, 2001; and Yang et al., Protein Eng. 16: 761-770, 2003). In one embodiment, antigenic epitopes can be identified and altered by mutagenesis. Methods of identifying antigenic epitopes are known in the art (see for example, Sette et al., Biologicals 29:271-276), as are methods of mutating such antigenic epitopes.

### 2. Preparation of Modified Pore-forming Protein Toxins

Modified pore-forming protein toxins (MPPTs) according to the present invention can be prepared by many methods, as known in the art. Modifications to the MPPT can be made, for example, by engineering the nucleic acid encoding the MPPT using recombinant DNA technology. Alternatively, modifications to the MPPT may be made by modifying the MPPT polypeptide itself, using chemical modifications and/or limited proteolysis. Combinations of these methods may also be used to prepare the MPPTs according to the present invention, as is also known in the art.

### 2.1 Preparation of MPPTs Using Recombinant Methods

As is known in the art, genetic engineering of a protein using recombinant DNA technology generally requires that the nucleic acid encoding the protein first be isolated and cloned. Sequences for various nPPTs are available from GenBank™ as noted herein. Isolation and cloning of the nucleic acid sequence encoding these proteins can thus be achieved using standard techniques [see, for example, Ausubel et al., Current Protocols in Molecular Biology, Wiley & Sons, NY (1997 and updates); Sambrook et al., supra]. For example, the nucleic acid sequence can be obtained directly from a suitable organism, such as Aeromonas hydrophila, by extracting the mRNA by standard techniques and then synthesizing cDNA from the mRNA template (for example, by RT-PCR) or by PCR-amplifying the gene from genomic DNA. Alternatively, the nucleic acid sequence encoding the nPPT can be obtained from an appropriate cDNA library by standard procedures. The isolated cDNA is then inserted into a suitable vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The vector may be a cloning vector or it may be an expression vector.

Once the nucleic acid sequence encoding the nPPT has been obtained, mutations in either the binding domains or activation sequence can be introduced at specific, pre-selected locations by in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

If desired, after introduction of the appropriate mutation or mutations, the nucleic acid sequence encoding the MPPT can be inserted into a suitable expression vector. Examples of suitable expression vectors include, but are not limited to, plasmids, phagemids, cosmids, bacteriophages, baculoviruses and retroviruses, and DNA viruses.

One skilled in the art will understand that the expression vector may further include regulatory elements, such as transcriptional elements, required for efficient transcription of the MPPT-encoding sequences. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered MPPT. One skilled in the art will appreciate that selection of suitable regulatory elements is dependent on the host cell chosen for expression of the genetically engineered MPPT and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed MPPT. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed MPPT prior to use according to methods known in the art. Alternatively, the amino acids corresponding to expression of heterologous nucleic acid sequences can be retained on the MPPT, providing that they do not interfere with the ability of the MPPT to target and kill cancer cells.

In one embodiment of the invention, the MPPT is expressed as a histidine tagged protein. The histidine tag is located at the carboxyl terminus of the MPPT.

The expression vectors can be introduced into a suitable host cell or tissue by one of a variety of methods known in the art. Such methods can be found generally described in Ausubel et al., Current Protocols in Molecular Biology, Wiley & Sons, NY (1997 and updates); Sambrook et al., Molecular Cloning: A Laboratory Manual, Cold-Spring Harbor Press, NY (2001) and include, for example, stable or transient transfection, lipofection, electroporation, and infection with recombinant viral vectors. One skilled in the art will understand that selection of the appropriate host cell for expression of the MPP will be dependent upon the vector chosen. Examples of host cells include, but are not limited to, bacterial, yeast, insect, plant and mammalian cells.

In addition, a host cell may be chosen which modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, and for post-translational modifications such as glycosylation and phosphorylation of the gene product can be used. Such mammalian host cells include, but are not limited to, CHO, VERO, BHK, HeLa, COS, MDCK, 293, 3T3, W138.

Methods of cloning and expressing proteins are well-known in the art, detailed descriptions of techniques and systems for the expression of recombinant proteins can be found, for example, in Current Protocols in Protein Science (Coligan, J. E., et al., Wiley & Sons, New York). Those skilled in the field of molecular biology will understand that a wide variety of expression systems can be used to provide the recombinant protein. The precise host cell used is not critical to the invention. Accordingly, the present invention contemplates that the MPPTs can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; or insect cells).

The MPPTs can be purified from the host cells by standard techniques known in the art. If desired, the changes in amino acid sequence engineered into the protein can be determined by standard peptide sequencing techniques using either the intact protein or proteolytic fragments thereof.

As an alternative to a directed approach to introducing mutations into naturally occurring pore-forming proteins, a cloned gene expressing a pore-forming protein can be subjected to random mutagenesis by techniques known in the art. Subsequent expression and screening of the mutant forms of the protein thus generated would allow the identification and isolation of MPPTs according to the present invention.

The MPPTs according to the present invention can also be prepared as fragments or fusion proteins. A fusion protein is one which includes an MPPT linked to other amino acid sequences that do not inhibit the ability of the MPPT to selectively target and kill normal cancer cells. In one embodiment, the other amino acid sequence encodes an ARD. In an alternative embodiment, the other amino acid sequences are short sequences of, for example, up to about 5, about 6, about 7, about 8, about 9, about 10, about 20, about 30, about 50 or about 100 amino acid residues in length.

Methods for making fusion proteins are well known to those skilled in the art. For example U.S. Pat. No. 6,057,133 discloses methods for making fusion molecules composed of human interleukin-3 (hIL-3) variant or mutant proteins functionally joined to a second colony stimulating factor, cytokine, lymphokine, interleukin, hematopoietic growth factor or IL-3 variant. U.S. Pat. No. 6,072,041 to Davis et al. discloses the generation of fusion proteins comprising a single chain Fv molecule directed against a transcytotic receptor covalently linked to a therapeutic protein.

Similar methods can be used to generate fusion proteins comprising MPPTs (or variants, fragments, etc. thereof) linked to other amino acid sequences, such as the ARDs described herein. Linker regions can be used to space the two portions of the protein from each other and to provide flexibility between them. The linker region is generally a polypeptide of between 1 and 500 amino acids in length, for example less than 30 amino acids in length. In general, the linker joining the two molecules can be designed to (1) allow the two molecules to fold and act independently of each other, (2) not have a propensity for developing an ordered secondary structure which could interfere with the functional domains of the two proteins, (3) have minimal hydrophobic or charged characteristic which could interact with the functional protein domains and/or (4) provide steric separation of the two regions. Typically surface amino acids in flexible protein regions include Gly, Asn and Ser. Other neutral amino acids, such as Thr and Ala, can also be used in the linker sequence. Additional amino acids can be included in the linker to provide unique restriction sites in the linker sequence to facilitate construction of the fusions. Other moieties can also be included, as desired. These can include a binding region, such as avidin or an epitope, or a tag such as a polyhistidine tag, which can be useful for purification and processing of the fusion protein. In addition, detectable markers can be attached to the fusion protein, so that the traffic of the fusion protein through a body or cell can be monitored conveniently. Such markers include radionuclides, enzymes, fluorophores, and the like.

Fusing of the nucleic acid sequences of the MPPT with the nucleic acid sequence of another protein (or variant, fragment etc. thereof), can be accomplished by the use of intermediate vectors. Alternatively, one gene can be cloned directly into a vector containing the other gene. Linkers and adapters can be used for joining the nucleic acid sequences, as well as replacing lost sequences, where a restriction site was internal to the region of interest. Genetic material (DNA) encoding one polypeptide, peptide linker, and the other polypeptide is inserted into a suitable expression vector which is used to transform prokaryotic or eukaryotic cells, for example bacteria, yeast, insect cells or mammalian cells. The transformed organism is grown and the protein isolated by standard techniques, for example by using a detectable marker such as nickel-chelate affinity chromatography, if a polyhistidine tag is used. The resulting product is therefore a new protein, a fusion protein, which has the MPPT joined to a second protein, optionally via a linker. To confirm that the fusion protein is expressed, the purified protein can be, for example, subjected to electrophoresis in SDS-polyacrylamide gels, and transferred onto nitrocellulose membrane filters using established methods. The protein products can be identified by Western blot analysis using antibodies directed against the individual components, i.e., polyhistidine tag and/or the MPPT.

If the MPPTs according to the present invention are produced by expression of a fused gene, a peptide bond serves as the linker between the MPPT and the ARD. For example, a recombinant fusion protein of a single chain Fv fragment of an antibody and a pore-forming protein toxin can be made according to methods known in the art, e.g., Huston et al., Meth. Enzymol. 203:46-88, 1991.

One of ordinary skill in the art will appreciate that the DNA can be altered in numerous ways without affecting the biological activity of the encoded protein. For example, PCR can be used to produce variations in the DNA sequence which encodes an MPPT. Such variations in the DNA sequence encoding an MPPT can be used to optimize for codon preference in a host cell used to express the protein, or may contain other sequence changes that facilitate expression.

### 2.2 Other Methods of Preparing MPPTs

The ARDs and linkers noted above may be added to the MPPTs of the present invention via a covalent or non-covalent bond, or both. Non-covalent interactions can be ionic, hydrophobic, or hydrophilic, such as interactions involved in a leucine-zipper or antibody-Protein G interaction (Derrick et al., Nature 359:752, 1992). Examples of additional non-covalent interactions include but are not restricted to the following binding pairs: antigen or hapten with antibody; antibody with anti-antibody; receptor with ligand; enzyme or enzyme fragment with substrate, substrate analogue or ligand; biotin or lectin with avidin or streptavidin; lectin with carbohydrate; pairs of leucine zipper motifs (see, for example, U.S. Pat. No. 5,643,731), as well as various homodimers and heterodimers known in the art. As is known in the art, the MPPT may be modified to include one member of the binding pair, and the ARD or linker may be modified to include the other member of the binding pair.

A covalent linkage may take various forms as is known in the art. For example, the covalent linkage may the form of a disulfide bond. The DNA encoding one of the components can be engineered to contain a unique cysteine codon. The second component can be derivatized with a sulfhydryl group reactive with the cysteine of the first component. Alternatively, a sulfhydryl group, either by itself or as part of a cysteine residue, can be introduced using solid phase polypeptide techniques. For example, the introduction of sulfhydryl groups into peptides is described by Hiskey (Peptides 3:137, 1981).

Proteins can be chemically modified by standard techniques to add a sulfhydryl group. For example, Traut's reagent (2-iminothiolane-HCl) (Pierce Chemicals, Rockford, Ill.) can be used to introduce a sulfhydryl group on primary amines, such as lysine residues or N-terminal amines. A protein or peptide modified with Traut's reagent can then react with a protein or peptide which has been modified with reagents such as N-succinimidyl 3-(2-pyridyldithio) propionate (SPDP) or succinimidyl 4-(N-maleimidomethyl)cyclohexane-1-carboxylate (SMCC) (Pierce Chemicals, Rockford, Ill.).

Once the correct sulfhydryl groups are present on each component, the two components are purified, sulfur groups on each component are reduced; the components are mixed; and disulfide bond formation is allowed to proceed to completion at room temperature. To improve the efficiency of the coupling reaction, the cysteine residue of one of the components, e.g., cysteine-MPPT, can be activated prior to addition to the reaction mixture with 5,5′-dithiobis(2-nitrobenzoic) acid (DTNB) or 2,2′-dithiopyridine, using methods known in the art. Following the reaction, the mixture is dialyzed against phosphate buffered saline to remove unconjugated molecules. Sephadex chromatography or the like is then carried out to separate the compound of the invention from its constituent parts on the basis of size.

The components can also be joined using the polymer, monomethoxy-polyethylene glycol (mPEG), as described in Maiti et al., Int. J. Cancer Suppl. 3:17-22, 1988.

The ARD and the nPPT or MPPT can also be conjugated through the use of standard conjugation chemistries as is known in the art, such as, carbodiimide-mediated coupling (for example, DCC, EDC or activated EDC), and the use of 2-iminothiolane to convert epsilon amino groups to thiols for crosslinking and m-maleimidobenzoyl-n-hydroxysuccinimidyl ester (MBS) as a crosslinking agent. Various other methods of conjugation known in the art can be employed to join the ARD and the nPPT or MPPT.

### 2.3 Large Scale Preparation of MPPTs

The preparation of the MPPTs can also be conducted on a large scale, for example for manufacturing purposes, using standard techniques known in the art, such as large scale fermentation processes for production of recombinant proteins, and ultrafiltration, ion exchange chromatography, immobilized metal ion affinity chromatography for purification of recombinant proteins.

### 3. Testing of Modified Pore-Forming Protein Toxins

The MPPTs according to the present invention retain their pore-forming activity, are activated by general or specific activating agents, and thus, are able to kill cancer cells. The ability of the MPPTs according to the present invention to kill cancer cells can be tested using standard techniques known in the art. Exemplary methods of testing candidate MPPTs are provided below and in the Examples included herein. One skilled in the art will understand that other methods of testing the MPPTs are known in the art and are also suitable for testing candidate MPPTs.

### 3.1 In Vitro Methods

MPPTs according to the present invention that contain one or more modifications to the activation sequence can be tested for their ability to be cleaved by the appropriate activating agent according to methods known in the art. For example, if the one or more modifications result in the addition of one or more protease cleavage sites, the MPPT can be incubated with varying concentrations of the appropriate protease(s). The incubation products can be electrophoresed on SDS-PAGE gels and cleavage of the MPPT can be assessed by examining the size of the polypeptide on the gel.

In order to determine if the MPPTs that have been incubated with protease retain pore-forming activity, and thus the ability to kill cells, after incubation with the protease, the reaction products can be tested in a hemolysis assay as is known in the art. An example of a suitable assay is described in Howard, S. P., and Buckley, J. T. 1985. Activation of the hole-forming toxin aerolysin by extracellular processing. J. Bacteriol. 163:336-340.

MPPTs according to the present invention can be tested for their ability to kill cancer cells as is known in the art. For example, the ability of the MPPTs to kill cells can be assayed in vitro using a suitable cell line, typically a cancer cell line. In general, cells of the selected test cell line are grown to an appropriate density and the candidate MPPT is added. After an appropriate incubation time (for example, about 48 to 72 hours), cell survival is assessed. Methods of determining cell survival are well known in the art and include, but are not limited to, the resazurin reduction test (see Fields & Lancaster (1993) Am. Biotechnol. Lab. 11:48-50; O'Brien et al., (2000) Eur. J. Biochem. 267:5421-5426 and U.S. Pat. No. 5,501,959), the sulforhodamine assay (Rubinstein et al., (1990) J. Natl. Cancer Inst. 82:113-118) or the neutral red dye test (Kitano et al., (1991) Euro. J. Clin. Investg. 21:53-58; West et al., (1992) J. Investigative Derm. 99:95-100) or trypan blue assay. Numerous commercially available kits may also be used, for example the CellTiter 96® AQueous One Solution Cell Proliferation Assay (Promega). Cytotoxicity is determined by comparison of cell survival in the treated culture with cell survival in one or more control cultures, for example, untreated cultures and/or cultures pre-treated with a control compound (typically a known therapeutic), or other appropriate control. MPPTs considered to be effective in killing cancer cells are capable of decreasing cell survival, for example, by at least 10%, at least 20%, at least 30%, at least 40%, or at least 50%.

MPPTs comprising optional modifications that confer selectivity for a specific type of cancer may be tested for their ability to target that specific cancer cell type. For example, an MPPT comprising an ARD that targets the MPPT to lung cancer cells can be assessed for its ability to selectively target lung cancer cells by comparing the ability of the MPPT to kill lung cancer cells to its ability to kill a normal cell, or a different type of cancer cell. Alternatively, flow cytometric methods, as are known in the art, may be used to determine if an MPPT comprising an ARD is able to selectively target a specific type of cancer cell.

Furthermore, MPPTs comprising one or more general cleavage sites, or a plurality of specific cleavage sites can be tested for their ability to be activated by the appropriate general or specific activating agent associated with cancer cells. As an example, the MPPT comprising a general cleavage site recognized by uPA can be incubated with a cancer cell that is known to express or activate uPA, followed by analysis of resulting cell death, usually expressed as cell killing curves. Non-limiting examples of cell lines that may be used to determine the ability of MPPTs to kill cancer cells associated with uPA include A2058 cells and HeLa cells. An example of a cell line expressing MMP2 is HT1080. In these types of experiments, EL4 cells can be used as a control cell line which is not associated with uPA or MMP2.

A variety of cancer cell-lines suitable for testing the candidate MPPTs are known in the art and many are commercially available (for example, from the American Type Culture Collection, Manassas, Va.). In one embodiment of the present invention, in vitro testing of the candidate compounds is conducted in a human cancer cell-line. In one embodiment of the present invention, in vitro testing of MPPTs is conducted in a human cancer cell-line. Examples of suitable cancer cell-lines for in vitro testing include, but are not limited to, mesothelial cell lines MSTO-21 1H, NCI-H2052 and NCI-H28, ovarian cancer cell-lines OV90 and SK-OV-3, breast cancer cell-lines MCF-7 and MDA-MB-231, colon cancer cell-lines CaCo, HCT116 and HT29, cervical cancer cell-line HeLa, non-small cell lung carcinoma cell-lines A549 and H1299, pancreatic cancer cell-lines MIA-PaCa-2 and AsPC-1, prostatic cancer-cell line PC-3, bladder cancer cell-line T24, liver cancer cell-lineHepG2, brain cancer cell-line U-87 MG, melanoma cell-line A2058, lung cancer cell-line NCI-H460. Other examples of suitable cell-lines are known in the art and include the EL4 mouse lymphoma cell line.

If necessary, the toxicity of the MPPTs to non-cancerous cells can also be initially assessed in vitro using standard techniques. For example, human primary fibroblasts can be transfected in vitro with the MPPTs and then tested at different time points following treatment for their viability using a standard viability assay, such as the assays described above, or the trypan-blue exclusion assay. Cells can also be assayed for their ability to synthesize DNA, for example, using a thymidine incorporation assay, and for changes in cell cycle dynamics, for example, using a standard cell sorting assay in conjunction with a fluorescence activated cell sorter (FACS).

### 3.2 In Vivo Methods

The ability of the MPPTs to kill tumor cells in vivo can be determined in an appropriate animal model using standard techniques known in the art (see, for example, Enna, et al., Current Protocols in Pharmacology, J. Wiley & Sons, Inc., New York, N.Y.).

Current animal models for screening anti-tumor compounds include xenograft models, in which a human tumor has been implanted into an animal. Examples of xenograft models of human cancer include, but are not limited to, human solid tumor xenografts, implanted by sub-cutaneous injection or implantation and used in tumor growth assays; human solid tumor isografts, implanted by fat pad injection and used in tumor growth assays; human solid tumor orthotopic xenografts, implanted directly into the relevant tissue and used in tumor growth assays; experimental models of lymphoma and leukemia in mice, used in survival assays, and experimental models of lung metastasis in mice. In addition to the implanted human tumor cells, the xenograft models can further comprise transplanted human peripheral blood leukocytes, which allow for evaluation of the anti-cancer immune response.

Alternatively, murine cancer models can be used for screening anti-tumor compounds. Examples of appropriate murine cancer models are known in the art and include, but are not limited to, implantation models in which murine cancer cells are implanted by intravenous, subcutaneous, fat pad or orthotopic injection; murine metastasis models; transgenic mouse models; and knockout mouse models.

For example, the MPPTs can be tested in vivo on solid tumors using mice that are subcutaneously grafted bilaterally with 30 to 60 mg of a tumor fragment, or implanted with an appropriate number of cancer cells, on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The MPPTs of the present invention can be administered to the animals, for example, by i.p. injection, intravenous injection, direct injection into the tumor, or bolus infusion. The different animal groups are weighed about 3 or 4 times a week until the maximum weight loss is attained, after which the groups are weighed at least once a week until the end of the trial.

The tumors are measured after a pre-determined time period, or they can be monitored continuously by measuring about 2 or 3 times a week until the tumor reaches a pre-determined size and/or weight, or until the animal dies if this occurs before the tumor reaches the pre-determined size/weight. The animals are then sacrificed and the tissue histology, size and/or proliferation of the tumor assessed.

Orthotopic xenograft models are an alternative to subcutaneous models and may more accurately reflect the cancer development process. In this model, tumor cells are implanted at the site of the organ of origin and develop internally. Daily evaluation of the size of the tumors is thus more difficult than in a subcutaneous model. A recently developed technique using green fluorescent protein (GFP) expressing tumors in non-invasive whole-body imaging can help to address this issue (Yang et al., Proc. Nat. Aca. Sci, (2000), pp 1206-1211). This technique utilises human or murine tumors that stably express very high levels of the Aqueora vitoria green fluorescent protein. The GFP expressing tumors can be visualised by means of externally placed video detectors, allowing for monitoring of details of tumor growth, angiogenesis and metastatic spread. Angiogenesis can be measured over time by monitoring the blood vessel density within the tumor(s). The use of this model thus allows for simultaneous monitoring of several features associated with tumor progression and has high preclinical and clinical relevance.

For the study of the effect of the compositions on leukemias, the animals are grafted with a particular number of cells, and the anti-tumor activity is determined by the increase in the survival time of the treated mice relative to the controls.

To study the effect of the MPPTs of the present invention on tumor metastasis, tumor cells are typically treated with the composition ex vivo and then injected into a suitable test animal. The spread of the tumor cells from the site of injection is then monitored over a suitable period of time.

In vivo toxic effects of the MPPTs can be evaluated by measuring their effect on animal body weight during treatment and by performing hematological profiles and liver enzyme analysis after the animal has been sacrificed.

TABLE 5
Examples of xenograft models of human cancer

| Cancer Model | Cell Type |
|---|---|
| Tumor Growth Assay | Prostate (PC-3, DU145) |
| Human solid tumor xenografts in mice (sub- | Breast (MDA-MB-231, MVB-9) |
| cutaneous injection) | Colon (HT-29) |
| | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa S3-HU- |
| | resistance) |
| | Liver (HepG2) |
| | Brain (U87-MG) |
| | Renal (Caki-1, A498) |
| | Ovary (SK-OV-3) |
| Tumor Growth Assay | Breast: drug resistant (MDA-CDDP-54, MDA- |
| Human solid tumor isografts in mice (fat pad | MB435-To.1) |
| injection) | |
| Survival Assay | Human: Burkitts lymphoma (Non-Hodgkin's) |
| Experimental model of lymphoma and | (raji) |
| leukemia in mice | Murine: erythroleukemia (CB7 Friend |
| | retrovirus-induced) |
| Experimental model of lung metastasis in mice | Human: melanoma (C8161) |
| | Murine: fibrosarcoma (R3) |

### 3.2.1 General Toxicity

The general toxicity of the MPPTs according to the present invention can be tested according to methods known in the art. For example, the overall systemic toxicity of the MPPTs can be tested by determining the dose that kills 100% of mice (i.e. LD₁₀₀) following a single intravenous injection.

### 3.3 Determination and Reduction of Antigenicity

Therapeutic proteins may elicit some level of antibody response when adminstered to a subject, which in some cases may lead to undesirable side effects. Therefore, if necessary, the antigenicity of the MPPTs can be assessed as known in the art and described below. In addition, methods to reduce potential antigenicity are described.

The kinetics and magnitude of the antibody response to the MPPTs described herein can be determined, for example, in immunocompetent mice and can be used to facilitate the development of a dosing regimen that can be used in a immunocompetent human. Immunocompetent mice such as the strain C57-BL6 are administered intravenous doses of MPPT. The mice are sacrificed at varying intervals (e.g. following single dose, following multiple doses).

To decrease antigenicity of MPPTs according to the present invention, the native binding domain of the MPPT can be functionally deleted and replaced, for example with an ARD as described above. The antigenicity of such MPPTs can be determined following exposure to varying schedules of the MPPT which lack portions of the native binding domain using the methods described above. Another method that can be used to allow continued treatment with MPPTs is to use sequentially administered alternative MPPTs derived from other nPPTs with non-overlapping antigenicity. For example, an MPPT derived from proaerolysin can be used alternately with an MPPT derived from Clostridium septicum alpha toxin or Bacillus thuringiensis delta-toxin. All of these MPPTs would target cancer cells, but would not be recognized or neutralized by the same antibodies.

Serum samples from these mice can be assessed for the presence of anti-MPPT antibodies as known in the art. As another example, epitope mapping can also be used to determine antigenicity of proteins as described in Marcia M. Stickler, David A. Estell and Fiona A. Harding. CD+ T cell epitope prediction using unexposed human donor peripheral blood mononuclear cells. J. Immunotherapy, 23(6):654-660, 2000. Briefly, immune cells known as dendritic cells and CD4+ T cells are isolated from the blood of community donors who have not been exposed to the protein of interest. Small synthetic peptides spanning the length of the protein are then added to the cells in culture. Proliferation in response to the presence of a particular peptide suggests that a T cell epitope is encompassed in the sequence. This peptide sequence can subsequently be deleted or modified in the MPPT thereby reducing its antigenicity.

### 4. Pharmaceutical Compositions

The present invention provides for pharmaceutical compositions comprising one or more MPPTs and one or more non-toxic pharmaceutically acceptable carriers, diluents, excipients and/or adjuvants. If desired, other active ingredients may be included in the compositions. As indicated above, such compositions are suitable for use in the treatment of cancer. The term “pharmaceutically acceptable carrier” refers to a carrier medium which does not interfere with the effectiveness of the biological activity of the active ingredients and which is not toxic to the host or patient. Representative examples are provided below.

The pharmaceutical compositions may comprise, for example, from about 1% to about 95% of a MPPT of the invention. Compositions formulated for administration in a single dose form may comprise, for example, about 20% to about 90% of the MPPTs of the invention, whereas compositions that are not in a single dose form may comprise, for example, from about 5% to about 20% of the MPPTs of the invention. Concentration of the MPPT in the final formulation can be as low as 0.01 μg/mL. For example, the concentration in the final formulation can be between about 0.01 μg/1 mL and about 1,000 μg/mL. In one embodiment, the concentration in the final formulation is between about 0.01 μg/mL and about 100 μg/mL. Non-limiting examples of unit dose forms include dragees, tablets, ampoules, vials, suppositories and capsules. Non-limiting examples of unit dose forms include dragees, tablets, ampoules, vials, suppositories and capsules.

The composition can be a liquid solution, suspension, emulsion, tablet, pill, capsule, sustained release formulation, or powder. For solid compositions (e.g., powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

For administration to an animal, the pharmaceutical compositions can be formulated for administration by a variety of routes. For example, the compositions can be formulated for oral, topical, rectal or parenteral administration or for administration by inhalation or spray. The term parenteral as used herein includes subcutaneous injections, intravenous, intramuscular, intrathecal, intrasternal injection or infusion techniques. Direct injection or infusion into a tumor is also contemplated. Convection enhanced delivery, a standard administration technique for protein toxins, is also contemplated by the present invention.

The MPPTs can be delivered along with a pharmaceutically acceptable vehicle. In one embodiment, the vehicle may enhance the stability and/or delivery properties. Thus, the present invention also provides for formulation of the MPPT with a suitable vehicle, such as an artificial membrane vesicle (including a liposome, noisome, nanosome and the like), microparticle or microcapsule, or as a colloidal formulation that comprises a pharmaceutically acceptable polymer. The use of such vehicles/polymers may be beneficial in achieving sustained release of the MPPTs. Alternatively, or in addition, the MPPT formulations can include additives to stabilise the protein in vivo, such as human serum albumin, or other stabilisers for protein therapeutics known in the art. MPPT formulations can also include one or more viscosity enhancing agents which act to prevent backflow of the formulation when it is administered, for example by injection or via catheter. Such viscosity enhancing agents include, but are not limited to, biocompatible glycols and sucrose.

Pharmaceutical compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavoring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed.

Pharmaceutical compositions for oral use can also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions formulated as aqueous suspensions contain the active compound(s) in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavoring agents or one or more sweetening agents, such as sucrose or saccharin.

Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavoring agents may be added to provide palatable oral preparations. These compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above. Additional excipients, for example, sweetening, flavoring and coloring agents, can also be included in these compositions.

Pharmaceutical compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavoring agents.

Pharmaceutical compositions can be formulated as a syrup or elixir by combining the active ingredient(s) with one or more sweetening agents, for example glycerol, propylene glycol, sorbitol or sucrose. Such formulations can also optionally contain one or more demulcents, preservatives, flavoring agents and/or coloring agents.

The pharmaceutical compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

In one embodiment, the MPPT is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. patent Pub. No. 20050106148 and references cited therein.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philidelphia, Pa. (2000).

The pharmaceutical compositions of the present invention described above include one or more MPPTs of the invention in an amount effective to achieve the intended purpose. Thus the term “therapeutically effective dose” refers to the amount of the MPPT that ameliorates the symptoms of cancer. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans, using standard methods known in those of ordinary skill in the art.

Therapeutic efficacy and toxicity can also be determined by standard pharmaceutical procedures such as, for example, by determination of the median effective dose, or ED₅₀ (i.e. the dose therapeutically effective in 50% of the population) and the median lethal dose, or LD₅₀ (i.e. the dose lethal to 50% of the population). The dose ratio between therapeutic and toxic effects is known as the “therapeutic index,” which can be expressed as the ratio, LD₅₀/ED₅₀. The data obtained from cell culture assays and animal studies can be used to formulate a range of dosage for human or animal use. The dosage contained in such compositions is usually within a range of concentrations that include the ED₅₀ and demonstrate little or no toxicity. The dosage varies within this range depending upon the dosage form employed, sensitivity of the subject, and the route of administration and the like.

The exact dosage to be administered to a subject can be determined by the practitioner, in light of factors related to the subject requiring treatment. Dosage and administration are adjusted to provide sufficient levels of the MPPT and/or to maintain the desired effect. Factors which may be taken into account when determining an appropriate dosage include the severity of the disease state, general health of the subject, age, weight, and gender of the subject, diet, time and frequency of administration, drug combination(s), reaction sensitivities, and tolerance/response to therapy. Dosing regimens can be designed by the practitioner depending on the above factors as well as factors such as the half-life and clearance rate of the particular formulation.

### 5. Use of Modified Pore-forming Toxins

### 5.1 Cancers

The MPPTs of the present invention can be used to treat, stabilize or prevent cancer. In this context, the MPPTs may exert either a cytotoxic or cytostatic effect resulting in, for example, a reduction in the size of a tumor, the slowing or prevention of an increase in the size of a tumor, an increase in the disease-free survival time between the disappearance or removal of a tumor and its reappearance, prevention of an initial or subsequent occurrence of a tumor (e.g. metastasis), an increase in the time to progression, reduction of one or more adverse symptom associated with a tumor, or an increase in the overall survival time of a subject having cancer.

Examples of cancers which may be may be treated or stabilized in accordance with the present invention include, but are not limited to, hematologic neoplasms, including leukemias, myelomas and lymphomas; carcinomas, including adenocarcinomas and squamous cell carcinomas; melanomas and sarcomas. Carcinomas and sarcomas are also frequently referred to as “solid tumors,” examples of commonly occurring solid tumors include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumor and, therefore, are also often considered to be solid tumors. In one embodiment of the present invention, the MPPTs are used to treat a solid tumor.

The term “leukemia” refers broadly to progressive, malignant diseases of the blood-forming organs. Leukemia is typically characterized by a distorted proliferation and development of leukocytes and their precursors in the blood and bone marrow but can also refer to malignant diseases of other blood cells such as erythroleukemia, which affects immature red blood cells. Leukemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukaemic leukemia, aleukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumors in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyte-rich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sezary Syndrome; precursor B-lymophoblastic lymphoma; B-cell small lymphocytic lymphoma; lymphoplasmacytic lymphoma; spenic marginal zome B-cell lymphoma; nodal marginal zome lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic T-cell lymphoma; and primary systemic anaplastic large T/null cell lymphoma.

The term “sarcoma” generally refers to a tumor which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate.

Additional cancers encompassed by the present invention include, for example, multiple myeloma, neuroblastoma, rhabdomyosarcoma, primary thrombocytosis, primary macroglobulinemia, small-cell lung tumors, primary brain tumors, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

In accordance with the present invention, the MPPTs can be used to treat various stages and grades of cancer development and progression. The present invention, therefore, contemplates the use of the MPPTs in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, the MPPTs may be used in the treatment of low grade cancers, intermediate grade cancers and or high grade cancers.

The present invention also contemplates that the MPPTs can be used in the treatment of indolent cancers, recurrent cancers including locally recurrent, distantly recurrent and/or refractory cancers (i.e. cancers that have not responded to treatment), metastatic cancers, locally advanced cancers and aggressive cancers. Thus, an “advanced” cancer includes locally advanced cancer and metastatic cancer and refers to overt disease in a patient, wherein such overt disease is not amenable to cure by local modalities of treatment, such as surgery or radiotherapy. The term “metastatic cancer” refers to cancer that has spread from one part of the body to another. Advanced cancers may also be unresectable, that is, they have spread to surrounding tissue and cannot be surgically removed.

One skilled in the art will appreciate that many of these categories may overlap, for example, aggressive cancers are typically also metastatic. “Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types.

The MPPTs may also be used to treat drug resistant cancers, including multidrug resistant tumors. As is known in the art, the resistance of cancer cells to chemotherapy is one of the central problems in the management of cancer.

Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the MPPTs in the treatment of such “hormone-resistant” or “hormone-refractory” cancers.

The present invention also contemplates the administration to a subject of a therapeutically effective amount of one or more MPPTs together with one or more anti-cancer therapeutics. The compound(s) can be administered before, during or after treatment with the anti-cancer therapeutic. An “anti-cancer therapeutic” is a compound, composition or treatment that prevents or delays the growth and/or metastasis of cancer cells. Such anti-cancer therapeutics include, but are not limited to, chemotherapeutic drug treatment, radiation, gene therapy, hormonal manipulation, immunotherapy and antisense oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C and the like. The compounds of the invention are also suitable for use with standard combination therapies employing two or more chemotherapeutic agents. It is to be understood that anti-cancer therapeutics for use in the present invention also include novel compounds or treatments developed in the future.

### 5.2 Administration

Typically in the treatment of cancer, MPPTs are administered systemically to patients, for example, by bolus injection or continuous infusion into a patient's bloodstream. Alternatively, the MPPTs may be administered locally, at the site of a tumor (intratumorally). When used in conjunction with one or more known chemotherapeutic agents, the compounds can be administered prior to, or after, administration of the chemotherapeutic agents, or they can be administered concomitantly. The one or more chemotherapeutics may be administered systemically, for example, by bolus injection or continuous infusion, or they may be administered orally.

In one embodiment, the MPPT can be injected into a subject having cancer, using an administration approach similar to the multiple injection approach of brachytherapy. For example, multiple aliquots of the purified MPPT in the form of a pharmaceutical composition or formulation and in the appropriate dosage units, may be injected using a needle. Alternative methods of administration of the MPPTs according to the present invention will be evident to one of skill in the art. Such methods include, for example, the use of catheters, or implantable pumps to provide continuous infusion of the MPPT to the subject in need of therapy.

As is known in the art, software planning programs can be used in combination with brachytherapy treatment and ultrasound, for example, for placement of catheters for infusing MPPTs to treat, for example, brain tumors or other localized tumors. For example, the positioning and placement of the needle can generally be achieved under ultrasound guidance. The total volume, and therefore the number of injections and deposits administered to a patient, can be adjusted, for example, according to the volume or area of the organ to be treated. An example of a suitable software planning program is the brachytherapy treatment planning program Variseed 7.1 (Varian Medical Systems, Palo Alto, Calif.). Such approaches have been successfully implemented in the treatment of prostate cancer, among others.

It is also contemplated that the MPPTs of the present invention can be co-administered with tracers in order to measure how the MPPT is distributed within the body after administration.

If necessary to reduce a systemic immune response to the MPPTs, immunosuppressive therapies can be administered in combination with the MPPTs. Examples of immunosuppressive therapies include, but are not limited to, systemic or topical corticosteroids (Suga et al., Ann. Thorac. Surg. 73:1092-7, 2002), cyclosporin A (Fang et al., Hum. Gene Ther. 6:1039-44, 1995), cyclophosphamide (Smith et al., Gene Ther. 3:496-502, 1996), deoxyspergualin (Kaplan et al., Hum. Gene Ther. 8:1095-1104, 1997) and antibodies to T and/or B cells [e.g. anti-CD40 ligand, anti CD4 antibodies, anti-CD20 antibody (Rituximab)] (Manning et al., Hum. Gene Ther. 9:477-85, 1998). Such agents can be administered before, during, or subsequent to administration of MPPTs according to the present invention).

The MPPTs may be used as part of a neo-adjuvant therapy (to primary therapy), as part of an adjuvant therapy regimen, where the intention is to cure the cancer in a subject. The present invention contemplates the use of the MPPTs at various stages in tumor development and progression, including in the treatment of advanced and/or aggressive neoplasias (i.e. overt disease in a subject that is not amenable to cure by local modalities of treatment, such as surgery or radiotherapy), metastatic disease, locally advanced disease and/or refractory tumors (i.e. a cancer or tumor that has not responded to treatment).

“Primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is begun soon after primary therapy to delay recurrence, prolong survival or cure a subject.

As noted above, it is contemplated that the MPPTs of the invention can be used alone or in combination with one or more other chemotherapeutic agents as part of an adjuvant therapy. Combinations of the MPPTs and standard chemotherapeutics may act to improve the efficacy of the chemotherapeutic and, therefore, can be used to improve standard cancer therapies.

This application can be particularly important in the treatment of drug-resistant cancers which are not responsive to standard treatment.

The dosage to be administered is not subject to defined limits, but it will usually be an effective amount. The compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The unit dosage forms may be administered once or multiple unit dosages may be administered, for example, throughout an organ, or solid tumor. Examples of ranges for the MPPT(s) in each dosage unit are from about 0.0005 to about 100 mg, or more usually, from about 1.0 to about 1000 μg.

Daily dosages of the compounds of the present invention will typically fall within the range of about 0.01 to about 100 mg/kg of body weight, in single or divided dose. However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended to limit the scope of the invention in any way. In some instances dosage levels below the lower limit of the aforesaid range may be more than adequate, while in other cases still larger doses may be employed without causing harmful side effects, for example, by first dividing the larger dose into several smaller doses for administration throughout the day.

### 6. Gene Therapy

The MPPTs according to the present invention, may also be employed in accordance with the present invention by expression of such proteins in vivo, which is often referred to as “gene therapy.”

Thus, for example, cells from a patient may be engineered with a polynucleotide (DNA or RNA) encoding an MPPT ex vivo, with the engineered cells then being provided to a patient to be treated with the polypeptide. Such methods are well-known in the art. For example, cells may be engineered by procedures known in the art by use of a retroviral particle containing RNA encoding an MPPT or a biologically active fragment thereof.

Similarly, cells may be engineered in vivo for expression of a polypeptide in vivo using procedures known in the art. As known in the art, a producer cell for producing a retroviral particle containing RNA encoding an MPPT, or a biologically active fragment thereof, may be administered to a patient for engineering cells in vivo and expression of the polypeptide in vivo. These and other methods for administering MPPTs by such methods should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses, from which the retroviral plasmid vectors hereinabove mentioned, may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequences encoding the MPPTs of the present invention are under the control of suitable promoters. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter which controls the genes encoding the MPPTs.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14×, VT-19-17-H2, ψCRE, ψCRIP, GP+E-86, GP+envAm12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or PTH to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

### 7. Clinical Trials

One skilled in the art will appreciate that, following the demonstrated effectiveness of an MPPT in vitro and in animal models, MPPTs should be tested in Clinical Trials in order to further evaluate its efficacy in the treatment of cancer and to obtain regulatory approval for therapeutic use. As is known in the art, clinical trials progress through phases of testing, which are identified as Phases I, II, III, and IV.

Initially MPPTs will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients is treated with a specific dose of an MPPT. During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to further evaluate the effectiveness and safety of an MPPT. In Phase II trials, an MPPT is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive MPPT treatment (investigational group).

Phase IV trials are used to further evaluate the long-term safety and effectiveness of a compound. Phase IV trials are less common than Phase I, II and III trials and will take place after the MPPT has been approved for standard use.

### 7.1 Eligibility of Patients for Clinical Trials

Participant eligibility criteria can range from general (for example, age, sex, type of cancer) to specific (for example, type and number of prior treatments, tumor characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of an MPPT and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

### 7.2 Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale. ECOG PS is a widely accepted standard for the assessment of the progression of a patient's disease as measured by functional impairment in the patient, with ECOG PS 0 indicating no functional impairment, ECOG PS 1 and 2 indicating that the patients have progressively greater functional impairment but are still ambulatory and ECOG PS 3 and 4 indicating progressive disablement and lack of mobility.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumor size.

### 7.3 Administration of MPPTs in Clinical Trials

MPPTs are typically administered to the trial participants parenterally. In one embodiment, an MPPT is administered by intravenous infusion. In another embodiment, an MPPT is administered intratumorally. Methods of administering drugs by intravenous infusion are known in the art. Usually intravenous infusion takes place over a certain time period, for example, over the course of 60 minutes.

A range of doses of an MPPT can be tested. An exemplary dose range for MPPT treatment includes dosages in the range 0.2 μg/kg body weight to 20 μg/kg body weight in single or divided doses.

### 7.4 Pharmacokinetic Monitoring

To fulfill Phase I criteria, distribution of the MPPT is monitored, for example, by chemical analysis of samples, such as blood or urine, collected at regular intervals. For example, samples can be taken at regular intervals up to until about 72 hours after the start of infusion. In one embodiment, samples are taken at 0, 0.33, 0.67, 1, 1.25, 1.5, 2, 4, 6, 8, 12, 24, 48 and 72 hours after the start of each infusion of the MPPT.

If analysis is not conducted immediately, the samples can be placed on dry ice after collection and subsequently transported to a freezer to be stored at −70° C. until analysis can be conducted. Samples can be prepared for analysis using standard techniques known in the art and the amount of the MPPT present can be determined, for example, by high-performance liquid chromatography (HPLC).

Pharmacokinetic data can be generated and analyzed in collaboration with an expert clinical pharmacologist and used to determine, for example, clearance, half-life and maximum plasma concentration.

### 7.5 Monitoring of Patient Outcome

The endpoint of a clinical trial is a measurable outcome that indicates the effectiveness of a compound under evaluation. The endpoint is established prior to the commencement of the trial and will vary depending on the type and phase of the clinical trial. Examples of endpoints include, for example, tumor response rate—the proportion of trial participants whose tumor was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilization—the proportion of trial participants whose disease has stabilized, for example, whose tumor(s) has ceased to grow and/or metastasize, can be used as an endpoint. Other endpoints include toxicity and quality of life.

Tumor response rate is a typical endpoint in Phase II trials. However, even if a treatment reduces the size of a participant's tumor and lengthens the period of disease-free survival, it may not lengthen overall survival. In such a case, side effects and failure to extend overall survival might outweigh the benefit of longer disease-free survival. Alternatively, the participant's improved quality of life during the tumor-free interval might outweigh other factors. Thus, because tumor response rates are often temporary and may not translate into long-term survival benefits for the participant, response rate is a reasonable measure of a treatment's effectiveness in a Phase II trial, whereas participant survival and quality of life are typically used as endpoints in a Phase II trial.

### 8. Pharmaceutical Kits

The present invention additionally provides for therapeutic kits or packs containing one or more of the MPPTs or a pharmaceutical composition comprising one or more of the MPPTs for use in the treatment of cancer. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration. The kit can optionally further contain one or more other therapeutic agents for use in combination with the MPPTs of the invention. The kit may optionally contain instructions or directions outlining the method of use or dosing regimen for the MPPTs and/or additional therapeutic agents.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient or applied to and mixed with the other components of the kit.

The components of the kit may also be provided in dried or lyophilized form and the kit can additionally contain a suitable solvent for reconstitution of the lyophilized components. Irrespective of the number or type of containers, the kits of the invention also may comprise an instrument for assisting with the administration of the composition to a patient. Such an instrument may be an inhalant, syringe, pipette, forceps, measured spoon, eye-dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Production of MPPT1

An MPPT according to one embodiment of the invention, MPPT1, which is a naturally occurring proaerolysin polypeptide comprising modification of the activation sequence to add a uPA site, and a histidine tag, was prepared as follows. Proaerolysin is normally activated by cleavage catalyzed by furin within the sequence between amino acids 427 and 432 (KVRRAR). In order to prevent furin activation, and to produce a product that can be activated by uPA, the furin cleavage sequence was replaced with the sequence SGRSAQ, which is known to be a substrate for uPA. This was accomplished by changing 3 codons at a time using the Quikchange II kit from Stratagene. In the first step, 2 complimentary primers were synthesized (5′-GCG GCT GAC AGC AGT GGG CGT CGT GCT C-3′ [SEQ ID NO:10] and 5′-AGC ACG ACG CCC ACT GCT GTC AGC CGC G-3′ [SEQ ID NO:11 ]), which replaced the KVR codons in the wild-type sequence with SGR codons in a PCR amplification using plasmid PA-His::pTZ18U as a template. This plasmid was prepared as follows. DNA encoding a His tag was added to the end of the aerA gene in plasmid pTZ18pNB5 (Diep, D. B., Lawrence, T. S., Ausio, J., Howard, S. P. and Buckley, J. T. 1998. Secretion and properties of the large and small lobe of the channel-forming toxin aerolysin. Mol. Microbiol. 30:341-352) using a Stratagene Quikchange II kit.

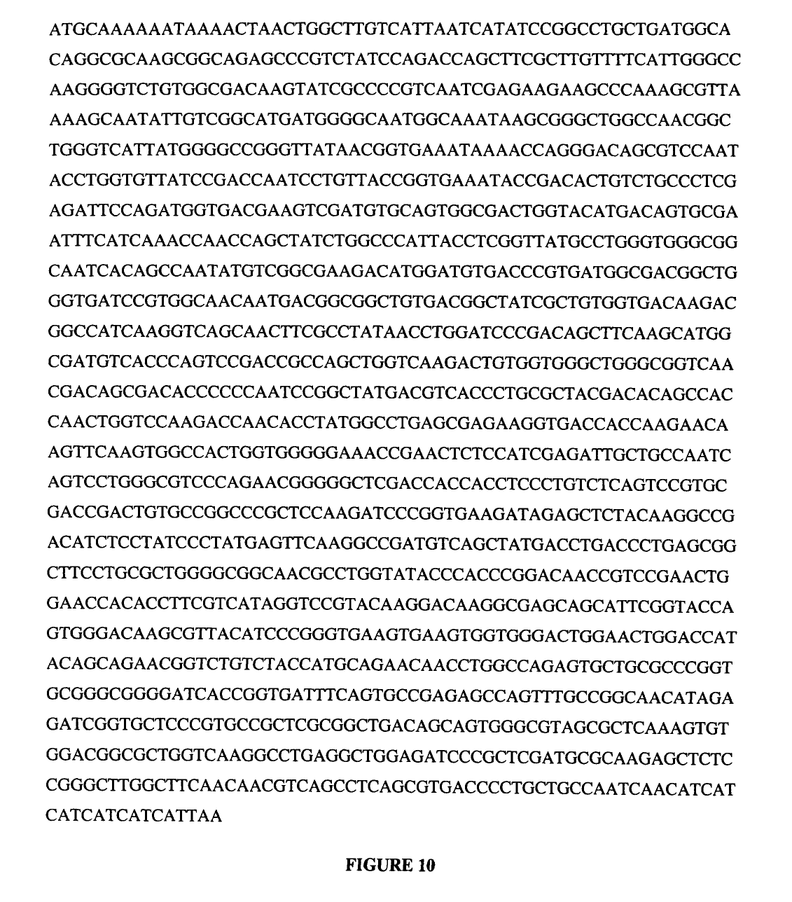

In a second PCR step, two more complimentary primers were synthesized (5′-AGC AGT GGG CGT AGC GCT CAA AGT GTG GAC G-3′ [SEQ ID NO:12] and 5′-GTC CAC ACT TTG AGC GCT ACG CCC ACT GCT G-3′ [SEQ ID NO:13]) which replace the last three codons of the target sequence, encoding RAR, to the codons for SAQ in a PCR amplification using the product from the first PCR as the template. The resulting construct, MPPT1, was excised with EcoRI and HindIII and ligated into the EcoRI and HindIII sites of the vector pMMB66HE. This plasmid, MPPT1::pMMB66, was transconjugated into Aeromonas salmonicida strain CB3 using the helper strain MM297-pRK2013 (Figurski and Helinski, 1989) as previously described (Wong et al., 1989). The resulting strain, CB3 MPPT1::pMMB66, was used to express MPPT1 for purification of the protein. The nucleotide sequence of MPPT1 (SEQ ID NO:20) is shown in FIG. 10.

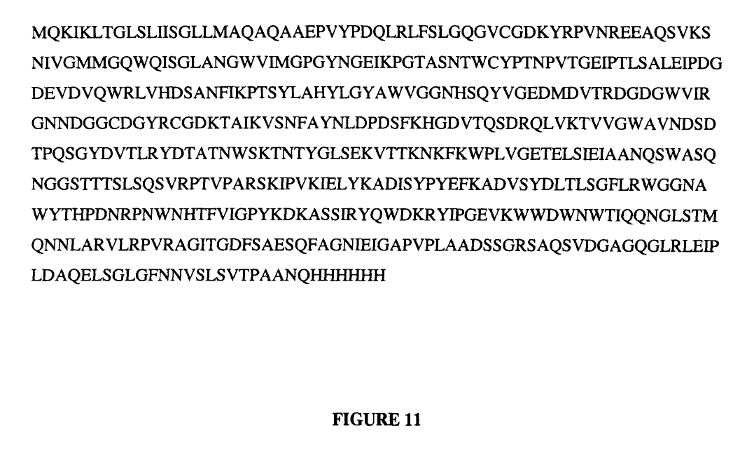

MPPT1 was expressed and purified as follows. CB3MPPT1::pMMB66 was inoculated into LB Davis media containing 0.2% glucose, 40 μg/ml rifampicin, 40 μg/ml kanamycin and 100 μg/ml ampicillin. and grown overnight at 27° C./250 rpm. It was then subinoculated (1%) into the same medium and incubated at 27° C./250 rpm. When the culture reached an OD_{600 nm} of approx. 0.7, IPTG was added to a final concentration of 1 mM to induce MPPT1 production and incubation was continued for 17.5 hours. The overnight cultures (OD_{600 nm}=4.5-4.8) were centrifuged at 10,000 rpm/15 minutes/4° C. in a JA-16.25 rotor (Beckman) and the culture supernatants were collected. The supernatant was concentrated from 2.4 L to ˜90 ml using a Kvick Lab SCU 10,000 Dalton cutoff membrane (Amersham; polyethersulfone membrane/0.11 m² filtration area) at 4° C. The concentrate was centrifuged at 10,000 rpm/4° C. using a JA-25.5 (Beckman) rotor for 10 minutes. The supernatant was loaded onto a 5 ml XK16 Ni²⁺ column (Chelating Sepharose Fast Flow, Amersham) equilibrated in 20 mM Na₂HPO₄, 0.5 M NaCl, 10 mM imidazole, pH 7.4 at 1 ml/min. After washing the column with 105 ml equilibration buffer, the column was eluted with 60 ml of 20 mM Na₂HPO₄, 0.5 M NaCl, 200 mM imidazole, pH 7.4 at 2.5 ml/min. The protein containing fractions were identified by measuring A₂₈₀ values of the fractions, and the peak tubes were loaded in 2.5 ml aliquots onto a PD10 column (Amersham) equilibrated in 10 mM NaH₂PO₄, 0.15 M NaCl, 1 mM EDTA, pH 7.4. Protein was eluted with 3.5 ml of the same buffer and the PD10 fractions were frozen and stored at −80° C. The amino acid sequence of MPPT1 (SEQ ID NO:21) is shown in FIG. 11.

### Example 2

### Production of MPPT2, and MPPT3

MPPTs according to additional embodiments of the invention were prepared. MPPT2 is a proaerolysin polypeptide with an activation sequence modified to add a uPA cleavage site, an ARD (specifically an AFAI antibody fragment), and a histidine tag. MPPT3 is a proaerolysin polypeptide with an activation sequence modified to add a uPA cleavage site, an ARD (specifically an AFAI antibody fragment) linked to the proaerolysin polypeptide with a linker that is cleavable by uPA, and a histidine tag. These MPPTs were prepared as follows.

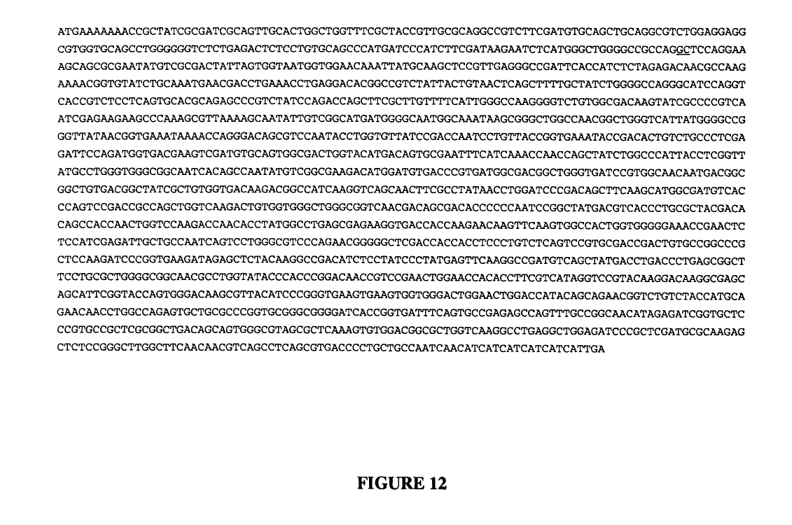

MPPT2 was prepared using the two sets of primers shown in the production of MPPT1 in Example 1 (SEQ ID NO:10-13) in a two-step Quikchange mutagenesis procedure (as described in Example 1) using the plasmid AFA-PA-His::pTZ18U as a template (see below for construction of this plasmid). The resulting plasmid was named MPPT2::pMMB67EH. The nucleotide sequence of MPPT2 (SEQ ID NO:22) is shown in FIG. 12.

AFA-PA-His::pTZ18U was prepared as follows. The starting point for production of the plasmid AFA-PA-His::pTZ18U was the construct encoding the AFA-large lobe of proaerolysin fusion described in International Patent Application No. PCT/CA2004/000309 (WO 2004/078097). It encodes 116 amino acids from AFA, preceded by an E. coli ompA signal sequence, and followed by amino acids 80-470 of proaerolysin. This construct was inserted into the vector pSJF2 and named AFA-PALLa::pSJF2. Six histidines codons were placed at the C-terminus of AFA-PALLa by a two-step Quikchange (Stratagene) procedure, adding 3 codons at each step. The resulting plasmid was called AFA-PALLa-His6::pSJF2.

In order to make a full length proaerolysin molecule with the AFA fragment fused to its N-terminus, AFA-PALLa-His6::pSJF2 was used as a template in a PCR mutagenesis procedure. One primer was synthesized (5′-ATA GAC GGG CTC TGC GTG CAC TGA GGA GAC G-3′ SEQ ID NO:53) and used with the pUC reverse primer and AFA-PALLa::pSJF2 plasmid to amplify a fragment that contained the ompA signal sequence and AFA portions of the construct. Wild-type proaerolysin was used as the template in a second PCR step. A second primer was synthesized (5′-GTC TCC TCA GTG CAC GCA GAG CCC GTC TAT C-3′ SEQ ID NO:54) and used with the pUC reverse primer to amplify the entire proaerolysin gene from the plasmid PA::pTZ18U (originally named pTZ18pNB5 in Diep, D. B., Lawrence, T. S., Ausio, J., Howard, S. P. and Buckley, J. T. 1998. Secretion and properties of the large and small lobe of the channel-forming toxin aerolysin. Mol. Microbiol. 30:341-352). When the two resulting PCR products were mixed together with the pUC reverse primer in a third PCR reaction, a product that contained the ompA signal sequence and AFA molecule fused to the entire proaerolysin molecule was formed. The ompA signal sequence/AFA/small lobe portion of this product was cut out with the restriction enzymes PstI and BamHI and ligated into a PstI/BamHI digested fragment of AFA-PALLa-His6::pSJF2. The resulting plasmid was called AFA-PA-H6::pSJF2. In order to use AFA-PA-His in subsequent cloning steps, the AFA-PA-His insert was cut out of the pSJF2 plasmid with the restriction enzymes EcoRI and HindIII and ligated into the EcoRI and HindIII sites of the cloning plasmid pTZ18U to produce AFA-PA-His::pTZ18U. The PCR product resulting from mutagenesis of AFA-PA-His::pTZ18U was cut with EcoRI and HindIII and ligated into the EcoRI and HindIII sites of the vector pMMB67EH.

MPPT3 was prepared as follows. An initial construct was prepared in which a thrombin cut-site (LVPRGS) had been engineered between AFA and the small lobe of proaerolysin (called AFA-t-PA::pTZ18U). This thrombin construct was engineered by adding codons for the first 3 amino acids of the cut-site (LVP) using the Quikchange Mutagenesis kit (Stratagene). Two primers were synthesized to insert these 3 amino acids:

[SEQ ID NO:14]

5′-GTCTCCTCAGTGCACCTAGTCCCTGCAGAGCCCGTCTATC-3′
(fwd)

[SEQ ID NO:15]

5′-ATAGACGGGCTCTGCAGGGACTAGGTGCACTGAGGAGACG-3′
(rev)

The product of this mutagenesis reaction was then used as the template for a second Quickchange mutagenesis step, in which 2 new primers were synthesized in order to insert the codons for the final three amino acids (RGS):

[SEQ ID NO:16]

5′-GTGCACCTAGTCCCTCGTGGTTCCGCAGAGCCCGTCTATC-3′
(fwd)

[SEQ ID NO:17]

5′-ATAGACGGGCTCTGCGGAACCACGAGGGACTAGGTGCACTG-3′
(rev)

This Quickchange mutagenesis product contained the thrombin cut-site between the AFA and proaerolysin portions of the protein. This product was digested with HindIII and EcoRI and ligated into the vector pTZ18U to form the plasmid AFA-t-PA::pTZ18U. This plasmid was used as the template for a one step mutagenesis protocol using the Quickchange mutagenesis (Stratagene) kit. The primers used in this mutagenesis were:

[SEQ ID NO:18]

5′-GTC TCC TCA GTG CAC TCA GGC CGT AGT GCT CAA
GCA GAGC-3′(fwd)

[SEQ ID NO:19]

5′-C TGG ATA GAC GGG CTC TGC TTG AGC ACT ACG GCC
TGA GTG CAC TGA GGA GAC_{-3′}(rev)

These primers changed the LVPRGS thrombin cut site to a SGRSAQ uPA cut-site in a single Quickchange (Stratagene) PCR step. The resulting PCR product was cut with EcoRI and HindIII and ligated into the EcoRI and HindIII sites of vector pMMB67EH. This plasmid was named AFA-uPA-PA-His::pMMB67EH.

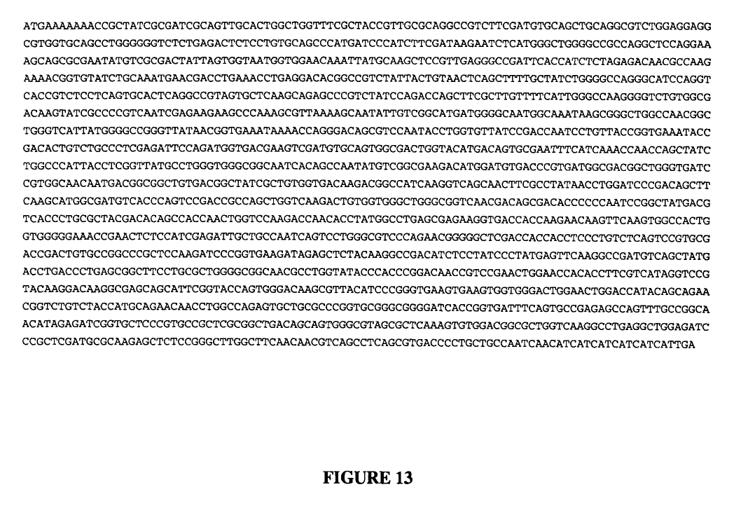

In order to prepare the MPPT3 construct, both AFA-uPA-PA-His::pTZ18U and MPPT2::pTZ18U plasmids were digested with KpnI and PstI restriction enzymes. A band of approximately 1.4 kb was purified from the AFA-uPA-PA-His::pTZ18U digest, while a band of approximately 3.3 kb was purified from the MPPT2::pTZ18U digest. These two fragments were ligated together to form the construct MPPT3::pTZ18U. This plasmid was digested with EcoRI and HindIII, and the MPPT3 insert was ligated into the EcoRI and HindIII restriction sites of vector pMMB67EH. The resulting plasmid was called MPPT3::pMMB67EH. The nucleotide sequence of MPPT3 (SEQ ID NO:24) is shown in FIG. 13.

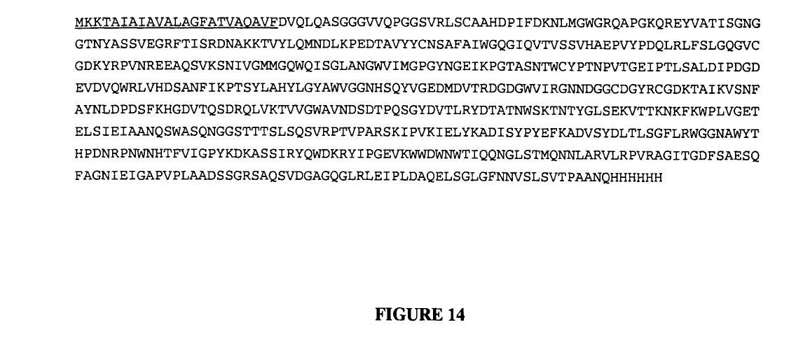

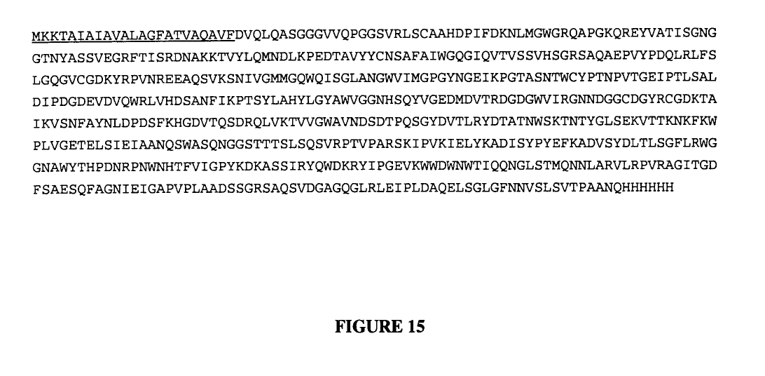

MPPT2::pMMB67EH and MPPT3::pMMB67EH were transconjugated into CB3 as described above. Strains CB3MPPT2::pMMB67EH, and CB3MPPT3::pMMB67EH were used to express and purify MPPT2 and MPPT3. These strains were each inoculated into 30 ml of LB Davis media containing 0.2% glucose, 40 μg/ml rifampicin, 40 μg/ml kanamycin and 100 μg/ml ampicillin. The cultures were grown overnight at 27° C./250 rpm. The overnight cultures were subinoculated and the purifications were carried out as described as described for MPPT1 in Example 1. The amino acid sequences of MPPT2 (SEQ ID NO:23) and MPPT3 (SEQ ID NO:25) are shown in FIGS. 14 and 15.

### Example 3

### Activation of MPPT1 by uPA-I

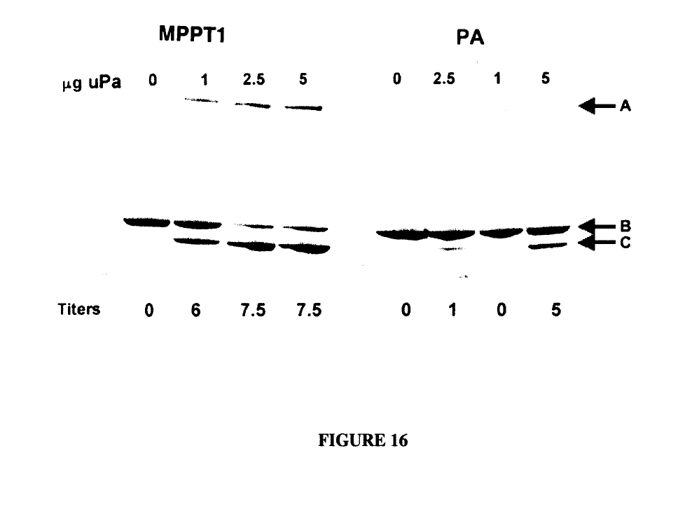

To test whether the MPPT1 could be activated by uPA, both MPPT1 and a control wild-type proaerolysin (PA) were incubated with varying concentrations of uPA (Sigma) and tested for their ability to lyse horse red blood cells. Protease digestion was carried out by incubating 4 μg of wild type proaerolysin or MPPT1 with 0, 1, 2.5, or 5 μg of uPA in a volume of 62.5 μL HBS (20 mM HEPES, 0.15 M NaCl, pH 7.4) for 60 min. at room temperature. The protease was inhibited by adding PMSF to 1 mM. After incubation, samples (2 μg) were also run on SDS polyacrylamide gels (10% NuPAGE Bis-Tris gels run in 1×MOPS buffer, Invitrogen) and stained with Coomasie Blue (FIG. 16).

The hemolysis assay was carried out as follows. Washed erythrocytes were prepared by diluting whole horse blood in phosphate buffered saline (PBS; 10 mM NaH₂PO₄, 0.15 M NaCl, pH 7.4) and centrifuging to pellet cells. The supernatant and white blood cells were removed and the erythrocytes were resuspended in PBS and pelleted again. This washing procedure was repeated until the supernatant was colorless. The packed cells were then suspended in HBS to 0.8% (v:v). To carry out the titer assay, 6 μg of test sample was added to the first row of 96 well titer plates and the volume was adjusted to 90 μl with HBS. The test samples were activated by adding 10 μl of protease, to obtain the indicated final protease concentration, and then incubating at room temperature for the required time. After incubation, 100 μl of HBS was added to the first well, and the samples were serially diluted 1:2 with HBS. After dilution, 100 μl of the 0.8% erythrocytes was added to each well and the plate was incubated at 37° C. The titer values were recorded at 5 minutes, 10 minutes, 15 minutes, 30 minutes and 60 minutes by visually assessing the number of wells showing lysis or clearing in each row.

The results are shown in FIG. 16, where A indicates oligomer; B indicates MPPT1; C indicates activated aerolysin. The results indicate that MPPT1 was much more readily activated by uPA than native PA was. Thus, the MPPT1 sample digested with 1 μg of uPA showed an activated aerolysin band (˜48 kDa; lowest band), and a high molecular weight oligomer band. No activated aerolysin band, and only a very faint high molecular weight oligomer band, was seen for the PA sample digested with 1 μg of uPA.

### Example 4

### Activation of MPPT2 and MPPT3

To test whether MPPT2 and MPPT3 could be activated upon digestion with uPA, 7.5 μg of each protein was incubated with 4 μg of uPA in a total of 100 μl of HBS (20 mM HEPES, 0.15 M NaCl, pH 7.4) for 4 hours at 37° C. The protease inhibitor phenylmethylsulfonylfluoride was then added to a final concentration of 1 mM to stop digestion. Samples (6 μg) were transferred to 96-well titer plates, and then assayed for hemolytic activity against horse red blood cells (0.4%) as described in Example 3.

TABLE 6
Hemolytic titers of proteins treated with uPA.

| Protein | Hemolytic activity after 60 minutes |
|---|---|
| MPPT2 | 4.5 |
| MPPT3 | 8 |

The results snow that MPPT3 was most active alter treatment with uPA, which can remove both the AFA portion and the C-terminus of the molecule, producing aerolysin. This also indicated that the presence of the AFA fusion molecule reduced the hemolytic activity of aerolysin (Table 6).

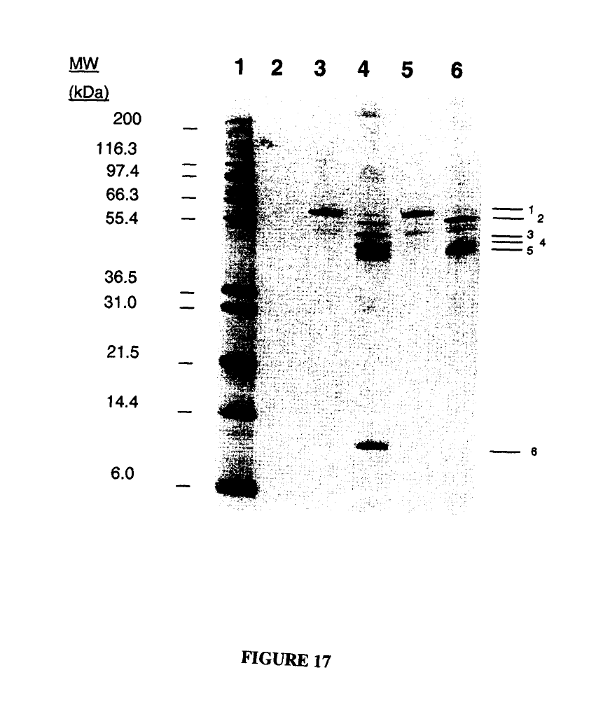

Each of the MPPTs was also examined by SDS-PAGE after digestion with uPA (see FIG. 17). Samples (7.5 μg) were incubated with (Lanes 4, 6) or without (Lanes 3, 5) 0.04 mg/ml urokinase (Sigma) at 37° C. for 4 hr before loading 100 ng of each sample onto a 10% NuPAGE Bis-Tris gel (Invitrogen) and running under non-reducing conditions. Lane 1, Molecular weight standards; Lanes 3 and 4, MPPT3; Lanes 9 and 10, MPPT2. Lane 2 is blank. Bands that are observed are the full length AFA-PA-His (1), AFA-PA, AFA-PA constructs with C-terminal peptide removed (2), AFA-PA with AFA portion removed (3), aerolysin (4), urokinase (5), and AFA that has been digested from the molecule (6). The results shown in FIG. 17 are consistent with the presence or absence of the uPA activation site in the variants. MPPT3 is the only polypeptide that produced a band corresponding to aerolysin after digestion, accounting for its activity in the titer assay.

### Example 5

### Ability of MPPT1, MPPT2 and MPPT3 to Kill A549 Human Lung Carcinoma Cells

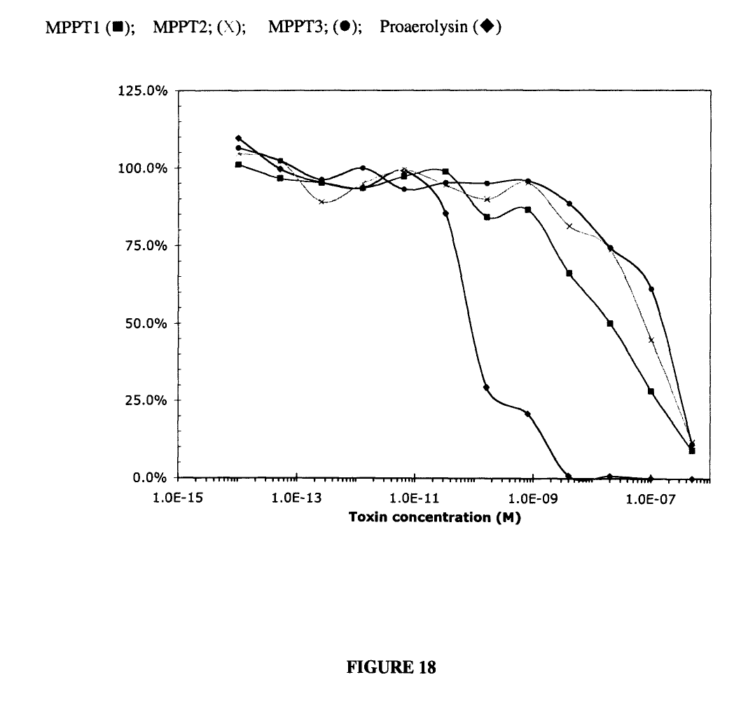

The toxicities of MPPT1, MPPT2, and MPPT3 were tested in a cell killing assay with the A549 cell line. Cell killing assays were performed with A549 cells that were grown in DMEM media (Gibco) containing 5% fetal bovine serum (FBS; Gibco) at 37° C./5% CO₂/48 hrs. Media was pipetted from the cell culture plate and the cells were rinsed 3 times with 5 ml of PBS (Gibco). The A549 cells were trypsinized with 3 ml of 0.05% trypsin in 0.53 mM EDTA for 2 min at 37° C./5% CO₂, and for 3 more minutes without trypsin, before being resuspended in fresh DMEM/5% FBS to give a cell concentration of 1.67×10⁵ cells/ml. Each well of a 96 well titer plate had 40 μl of DMEM/5% FBS media added to it. Sample was added (10 μl) to the first well in each of the first 7 rows of the plate, mixed by gently pipetting the contents of the well up and down, and 10 μl was transferred to the next well. This serial 1:5 dilution was continued along the entire row, and then 60 μl of A549 was added to each well of 7 rows of the plate. The 8^{th} row was a no-cell negative control. The plate was incubated at 37° C./5% CO₂ for 1 h. 20 μl of Cell Titer 96 Aqueous One Solution Reagent (Promega) was added to each well, and incubation continued for 3 more hours at 37° C./5% CO₂. The absorbance at 490 nm was read for each well, and the cell viability was calculated by subtracting the A₄₉₀ nm value of the negative control. The results in FIG. 18 show that these MPPT1, MPPT2, and MPPT3 were much less active than native PA, but still able to kill A549 cells.

### Example 6

### In Vivo Efficacy of MPPT1 and MPPT2 in A549 Human Lung Carcinoma Xenograft Tumors

The ability of MPPTs according to the present invention to reduce tumor growth in mice was determined using an A549 human lung carcinoma xenograft model as follows. On study day 0, 44 female Rag2m mice (T & B cell deficient) were inoculated subcutaneously on the back with 2×10⁶ A549 human lung carcinoma cells. When average tumor size was 200 mm³, mice were treated intratumorally with test compound at a dose of 5 μg/mouse in 25 μL of phosphate-buffered saline, 1 mM EDTA, pH 7.4. Mice were monitored for tumor size and for tumor or treatment-related morbidity/mortality (body weight changes; altered gait, lethargy, gross magnifications of stress) over the course of the study. Mice with ulcerated tumors, or tumors in excess of 1000 mm³ were terminated by CO₂ asphyxiation.

### Test Compound Coding

| | |
|---|---|
| MPPT1 | Proaerolysin containing a uPA selective activation site |
| MPPT2 | Proaerolysin containing a uPA selective activation site and |
| | with AFA antibody attached |
| Control 1 | Native proaerolysin |
| Control 2 | PBS/EDTA buffer |

All of the test compounds have a His tag at the C-terminus of proaerolysin.

### Method of Group Assignment

Mice were randomized to groups on the basis of tumor volume.

### Results

### Test Compound Administration

One mouse in group two had 35 μL injected rather than the required 25 μL due to some leakage of test compound that occurred during the injection. All other mice had test compounds administered as per the study protocol. No adverse reactions were noted for five of the test compounds, however 3 of the 5 mice injected with native proaerolysin were found dead within two days.

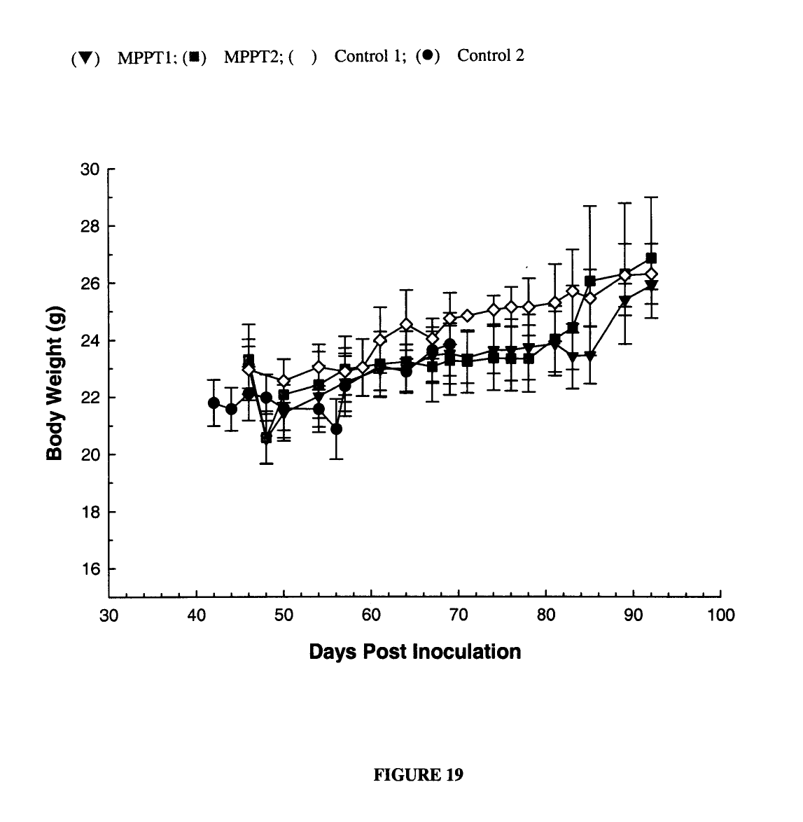

FIG. 19 shows the effect on body weight of mice treated with MPPTs. Although there were no significant differences in body weights between groups, a decrease in average body weight was noted in mice receiving MPPT1 and MPPT2 two days following test compound administration (12% loss). Average body weight in these groups had recovered to within 5% of initial by day 8 (MPPT1) or day 4 (MPPT2) post administration and no further loss was noted.

### Change in Tumor Volumes

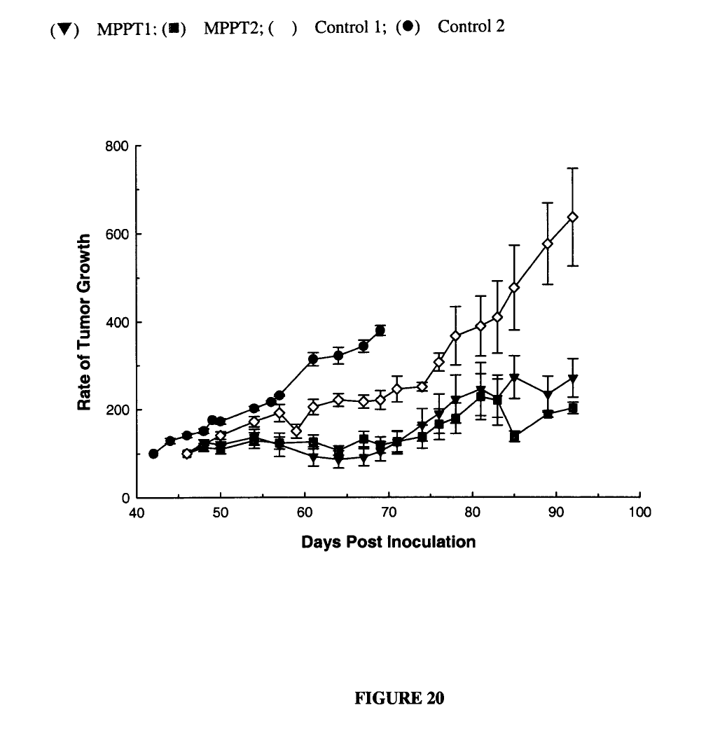

Administration of both MPPT1 and MPPT2 resulted in significant reduction in growth rate of A549 tumors compared to both the rate of growth of the PBS-EDTA injected control group and to the growth of tumors injected with proaerolysin (Control 1), as shown in FIG. 20. Growth rate was expressed as tumor volume divided by initial tumor volume, multiplied by 100.

### Observations

### Necropsy Results

Small spleens were noted on termination in two mice from the group receiving MPPT2.

### CONCLUSIONS

Except for proaerolysin itself, test compounds were well tolerated when administered intratumorally. Proaerolysin with the native furin activation site will be toxic to most cells, because the cells display furin on their surfaces. MPPTs containing the uPA or proaerolysin polypeptide containing a PSA activation sequence are not activated by furin and would be expected to be less toxic than native proaerolysin in the absence of uPA or PSA.

Intratumoral administration of MPPT1 or MPPT2 resulted in a significant reduction in the rate of subcutaneous A549 xenograft tumor growth in female Rag2m mice. This is consistent with the evidence that both uPA and the uPA receptor are upregulated in A549 tumors (1).

### REFERENCES

1. Marshall B. C, Xu, Q. P, Rao, N. V, Brown, B. R, and Hoidal, J. R. (1992 Pulmonary epithelial cell urokinase-type plasminogen activator. Induction by interleukin-1 beta and tumor necrosis factor-alpha. J. Biol. Chem. 267:11462-9.

### Example 7

### Activation of MPPT1 by uPA-II

The ability of MPPT1 to be activated by uPA was determined again by methods similar to those described in Example 3. Both PA and MPPT1 were incubated at 0.4 mg/mL with 0, 0.016 mg/mL, 0.04 mg/mL or 0.08 mg/mL uPA in HBS, pH 7.4 for 120 minutes at room temperature, in a total volume of 62.5 μL. After the incubation, 2 μg of each sample was removed and immediately loaded in 1×SDS-PAGE sample buffer onto 10% Bis-Tris NuPAGE gels in 1×MOPS-SDS running buffer under non-reducing conditions and run at 200 V constant voltage for 50 minutes followed by staining in Coomassie Brilliant Blue stain.

Also, 4 μg of each sample was removed and inhibited in a final volume of 100 μl BBS with 1 mM PMSF for a hemolytic titer assay. The hemolytic titer assay was carried out using a protocol similar to that described in Example 3. After serially diluting 1:2 in HBS across a 96 well plate, the samples were titered with 0.4% horse red blood cells (final concentration) incubated at 37° C. for 1 hour. Titer, or number of wells containing lysed cells, was estimated visually.

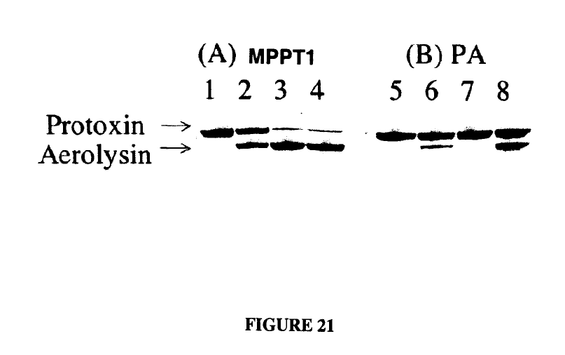

The results indicated that both PA and MPPT1 are activated by uPA, but MPPT1 is significantly more sensitive as seen in FIG. 21. FIG. 21 shows a Coomassie stained SDS-PAGE gel of urokinase digest of PA and MPPT1 after 120 minutes incubation at room temperature. Each lane contains 2 μg of toxin. Lanes: 1, MPPT1 undigested; 2, MPPT1 with 0.016 mg/mL uPA; 3, MPPT1 with 0.04 mg/mL uPA; 4, MPPT1 with 0.08 mg/mL uPA; 5, PA undigested; 6, PA with 0.04 mg/mL uPA; 7, PA with 0.016 mg/mL uPA; 8, PA with 0.08 mg/mL uPA. After 120 minutes, the MPPT1 is almost completely activated by 0.04 mg/mL uPA, whereas much less PA was activated. No PA was converted to aerolysin when treated with 0.016 mg/mL uPA under these conditions, whereas MPPT1 was nearly 50% converted.

The results of the corresponding hemolytic titer assay after 60 minutes of incubation are displayed in Table 7 below. As noted above, the hemolytic titers of PA and MPPT1 digested with a range of uPA concentrations for 120 minutes at room temperature were measured. Titers were read after 60 minutes incubation at 37° C. These data confirm the data shown in FIG. 21. No activation of PA by treatment with 0.016 mg/ml uPA was detected, yet MPPT1 was nearly 50% activated.

TABLE 7
Horse red blood cells hemolytic titers of PA and MPPT1 after 120
minutes of incubation

| | Hemolytic titer | | | |
|---|---|---|---|---|
| | | | 0.04 | 0.08 |
| | Unactivated | 0.016 mg/mL uPA | mg/mL uPA | mg/mL uPA |
| PA | 0 | 0 | 2.5 | 6 |
| MPPT1 | 0 | 6 | 7.5 | 7.5 |

### Example 8

### Ability of MPPT1 to Kill Cells Expressing Urokinase-Type Plasminogen Activator-Receptor (uPAR)

The toxicity of MPPT1 towards uPAR expressing cells was determined by testing the ability of MPPT1 to kill HeLa cells or A2058 cells. Two 96 well Costar titer plates containing either HeLa or A2058 cells at an estimated 50% confluency were prepared for each adherent cell line. Since A2058 cells grow faster than HeLa cells, it was likely that there were more cells in the A2058 plates.

The plates were washed twice in warmed plain DMEM media by pipetting off old media and replacing with 100 μl new media. The cells were preincubated with or without 0.1 μg/mL pro-urokinase and 1 μg/mL glu-plasminogen in a final volume of 240 μl media for 30 minutes at 37° C., 5% CO₂ with humidity. Cells that express the urokinase receptor will activate pro-urokinase under these conditions. Either PA or MPPT1 (1 mg/mL and 0.82 mg/mL, respectively) was added to the first well in each row, excluding those used for negative/negative and positive/negative controls, which received media to bring the volumes to 250 μL. The wells were serially diluted 1:5 in media with or without pro-urokinase and glu-plasminogen across the titer plate (200 μL remaining in each well). The plates were incubated for 1 hour at 37° C., 5% CO₂ with humidity.

To develop the plates, the old media was removed and 200 μL fresh media containing FBS was added back in addition to 50 μL of 2.5 mg/mL MTT. The plates were incubated as above for 80 minutes until purple precipitate became visible at the bottom of the wells.

The media was carefully removed without disturbing the precipitate and 100 μL of solubilization buffer (0.5% (w/v) SDS and 25 mM HCl in 90% (v/v) isopropanol) was added to each well. The openings of the wells were sealed with aluminum sealing foil and the plates were gently agitated at room temperature to solubilize the precipitate. The plates were measured at 560 nm using a 96 well plate reader and the raw data was used to generate cell killing curves of the toxin concentration vs cell viability.

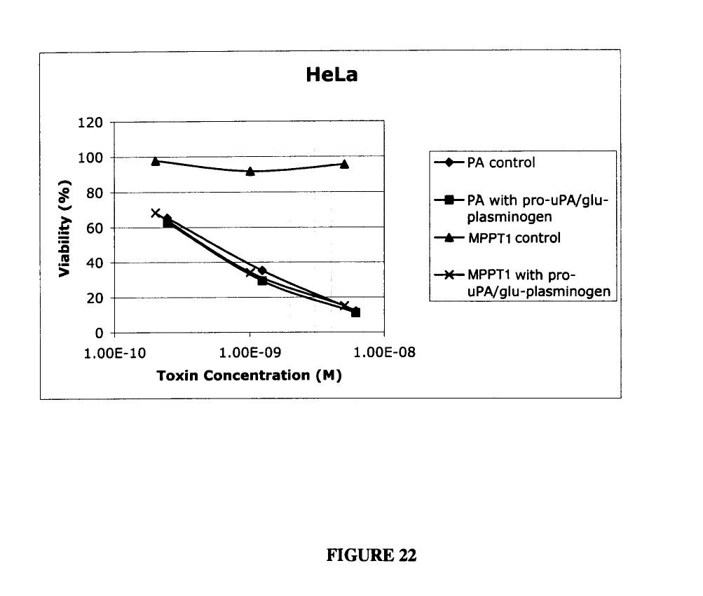

The results of the HeLa cell killing assay are shown in FIG. 22. The cell killing curve, as seen in FIG. 22, shows that the preincubation of uPAR-expressing HeLa cells with pro-uPA and glu-plasminogen (which results in the production of active uPA) enables MPPT1 to kill the cells as effectively as PA. Without the preincubation step, the MPPT1 shows little cell killing activity. This is because the normal furin activation site has been removed.

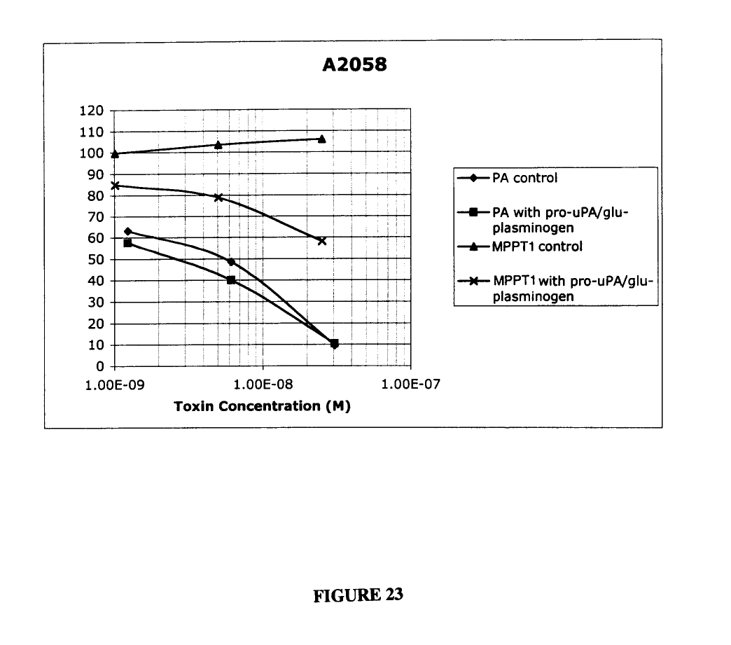

Similarly, as shown in FIG. 23, the cell killing curve for A2058 cells indicates that the preincubation of A2058 cells with pro-uPA and glu-plasminogen increases the toxicity of MPPT1, although the uPA-sensitive toxin was not able to kill as efficiently as PA.

### Example 9

### Ability of MPPT1 to Kill EL4 Mouse Lymphoma Cells

The toxicity of MPPT1 towards non-uPAR expressing cells was determined by testing its ability to kill EL4 cells as follows. EL4 cells were prepared to 1×10⁶ cells/mL suspended in media with or without 0.1 μg/mL pro-urokinase and 1 μg/mL glu-plasminogen and incubated at 37° C., 5% CO₂ with humidity for 30 minutes. The PA and MPPT1 (1 mg/mL and 0.82 mg/mL, respectively) were serially diluted 1/5 in triplicate across two 96 well plates in 20 μL media. The pre-incubated cells were added to each well (80 μL), excluding negative control and the plates were further incubated for 1 hour as above. To develop the assay, 20 μL Promega cell killing media (CellTiter 96AQueous One Solution Cell Proliferation Assay) was added to each well and incubated further for 4 hours as above. The bubbles were popped in each well by the addition of 7 μL 70% isopropanol and the plates were measured at 490 nm using a Bio-Tek plate reader. The raw absorbance data was used to produce cell killing curves of toxin concentration vs. the percentage of viable cells.

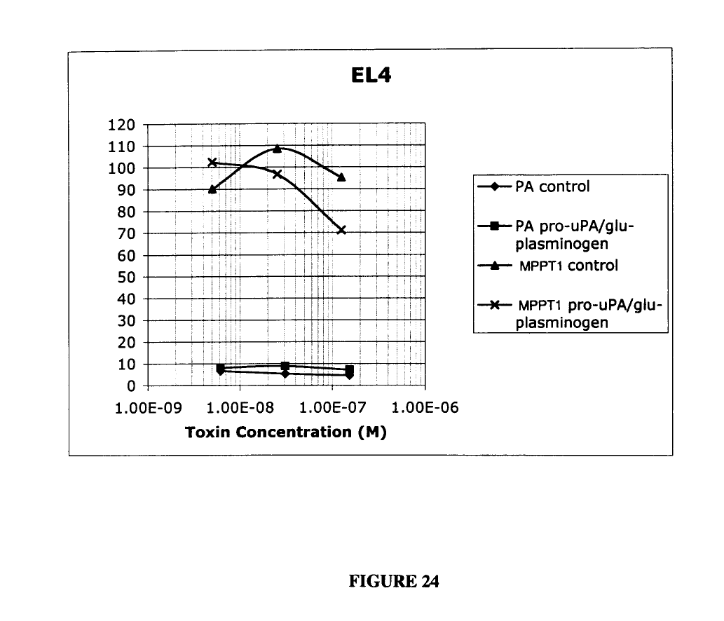

FIG. 24 depicts the cell killing curve for EL4 cells, and indicates that preincubation of the cells with pro-uPA and glu-plasminogen does not lead to activation of MPPT1. This is a necessary control, since EL4 cells do not express the uPA receptor needed to generate active uPA under these conditions.

The data shown in Examples 7 to 9 indicate that replacing the native furin activation site of PA with one that is specific for urokinase creates a variant, MPPT1, that is toxic to cells expressing urokinase receptors when pro-urokinase and glu-plasminogen are present.

### Example 10

### Production of an MPPT (MPPT4) that can be Activated by Matrix Metalloproteinase 2 (MMP2)

MPPT4, derived from proaerolysin and containing an activation sequence modified to include a single cleavage site cleavable by a general activating agent was prepared by replacing the native furin activation site with a sequence cleavable by MMP2. The MPPT4 was made by 3-step PCR and purified using nickel affinity and anion exchange chromatography as described below.

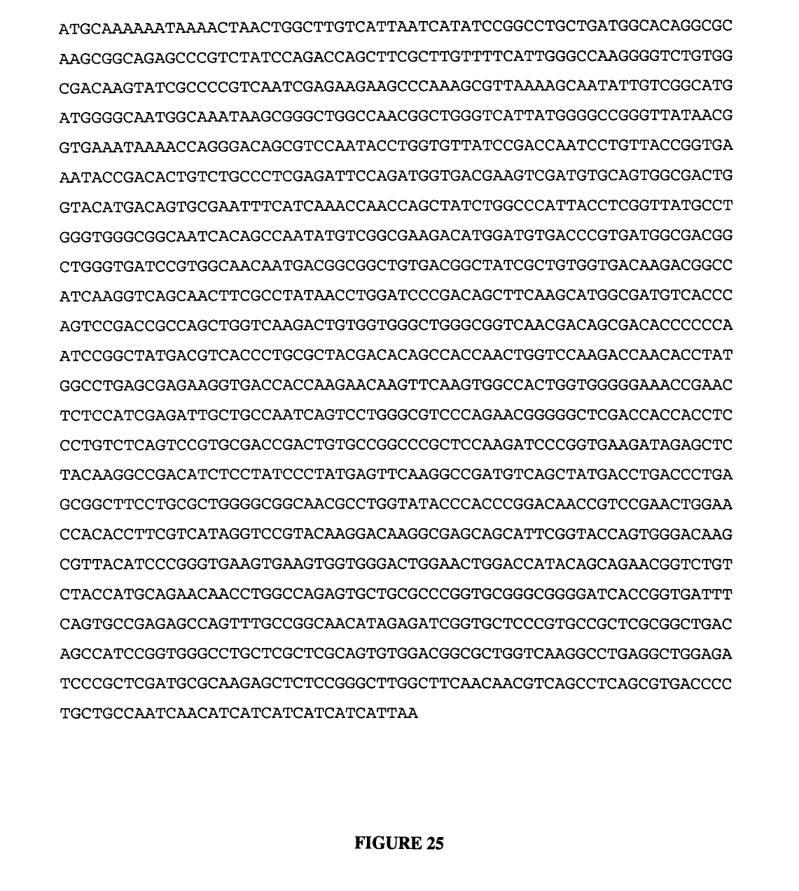

Cloning of MPPT4, derived from proaerolysin and having the activation sequence (KVRRAR) changed to that of MMP2 (HPVGLLAR) was prepared by three rounds of Site Directed Mutagenesis (SDM) using the Quick Change Kit (Stratagene) according to the manufacturer's instructions. In the first SDM, plasmid pTZ18U containing the gene coding for aerolysin with a 6×His tag at its C-terminus was used as a template, with the pair of primers MMP2-1fwd and MMP2-1rev (Table 8). The second SDM was performed using the product of the first SDM as a template and the pair of primers—MMP2-2fwd and MMP2-2rev (Table 8). The third SDM was performed using the product of the second SDM as a template and the pair of primers MMP2-3fwd and MMP2-3rev (Table 8). After the desired changes in the final product were confirmed by DNA sequencing, the mutated aerA gene was cloned (as HindIII-EcoRI fragment) into the broad host-range expression vector pMMB66HE. Recombinant clones were then transferred into Aeromonas salmonicida CB3 cells by conjugation using the filter-mating technique. The nucleotide sequence of MPPT4 (SEQ ID NO:38) is shown in FIG. 25.

TABLE 8
Primers used in Quick Change reactions

| | | SEQ ID |
|---|---|---|
| Primer | Sequence | No |
| MMP2-1fwd | CTCGCGGCTGACAGCCATCCGGTGCGTGCTCGCA | 26 |
| | G | |
| MMP2-1rev | CTGCGAGCACGCACCGGATGGCTGTCAGCCGCGA | 27 |
| | G | |
| MMP2-2fwd | GACAGCCATCCGGTGGGCCTGCTCAGTGTGGACG | 28 |
| | GC | |
| MMP2-2rev | GCCGTCCACACTGAGCAGGCCCACCGGATGGCTG | 29 |
| | TC | |
| MMP2-3fwd | CCGGTGGGCCTGGTCGCTCGCAGTGTGGACGGC | 30 |
| MMP2-3rev | GCCGTCCACACTGCGAGCGAGCAGGCCCACCGG | 31 |

MPPT4 was expressed and purified as follows. A single colony of CB3MPPT4::pMMB66 (CB3 strain containing a plasmid expressing MPPT4) was inoculated into 30 mL of LB media (10% Bacto Tryptone, 5% Bacto Yeast Extract, 10% NaCl, pH 7.5) containing Davis minimal media, 0.2% glucose, 100 μg/mL ampicillin and 40 μg/mL of both kanamycin and rifampicin. The culture was grown at 27° C. with shaking at 250 rpm overnight to an OD_{600 nm} of 3.76. The culture was inoculated (1%) into 5×2 L flasks containing a final volume of 500 mL of the above media excluding rifampicin. Cultures were grown as above for 5.25 hours to OD_{600 nm} from 0.59-0.68 and induced with a final concentration of 1 mM IPTG. The induced cultures were incubated as above for 17.5 hours. The culture supernatant was harvested by pelleting the cells at 10,000 rpm in a JA16.25 rotor for 15 minutes at 4° C. A few drops of polypropylene glycol were added to the supernatant to prevent frothing during concentration with a KVICK SCU concentrator (Amersham, 10,000 kDa cutoff). The supernatant was concentrated from 2.5 L to 80 mL and aliquoted in 20 mL fractions for storage at −20° C.

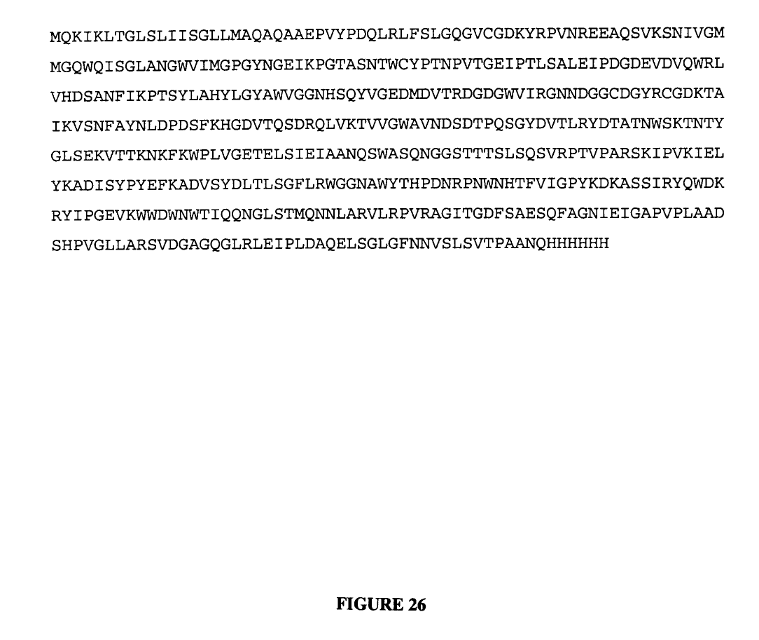

An aliquot of MPPT4 supernatant was thawed and clarified by centrifugation in a JA25.5 rotor at 10,000 rpm for 10 minutes at 4° C. The clarified supernatant was loaded at 1 m/min onto a 10 mL FPLC nickel affinity column (Amersham Chelating Sepharose Fast Flow resin) equilibrated in Buffer A (20 mM Na₂HPO₄, 0.5M NaCl, 10 mM imidazole, pH 7.4). The column was washed at 5 mL/min with 50 mL of Buffer A and eluted with 60% Buffer B (20 mM Na₂HPO₄, 0.5M NaCl, 0.5M imidazole, pH 7.4) at approximately 300 mM imidazole. The toxin containing fractions were desalted into 20 mM Tris, 1 mM EDTA, 150 mM NaCl, pH 7.4 using PD-10 exchange columns (Amersham). The desalted material was loaded at 1 mL/min onto an FPLC HiPrep 16/10 Q FF anion exchange column (Amersham) equilibrated in the same buffer as used for desalting. The column was washed in equilibration buffer and eluted with a linear gradient of 0.15-1 M NaCl in 20 mM Tris, 1 mM EDTA, pH 7.4. Concentrations were determined by measuring absorbance at 280 nm. The purification yielded pure protein as seen on Coomassie stained SDS-PAGE (not shown). The total yield of protein was 8.6 mg from 625 mL culture supernatant. The amino acid sequence of MPPT4 (SEQ ID NO:39) is shown in FIG. 26.

### Example 11

### Sensitivity of MPPT4 to Cleavage by MMP2

The sensitivity of MPPT4 to MMP2 was demonstrated using silver stained SDS-PAGE and Western blotting as follows. Both wild type PA and MPPT4 were incubated at a concentration of 0.02 mg/mL with 0, 0.5 μg, 0.25 μg, 0.125 μg and 0.0625 μg of active MMP2 protease (EMD/Calbiochem) in HBS, 10 mM CaCl₂, pH 7.4 for 3 hours, 24 hours and 48 hours at 37° C. Digestion was stopped with a final concentration of 1 μM 1,10-o-phenanthroline followed by chilling on ice.

Samples were prepared for electrophoresis by removing 10 μL inhibited material into 5 μL dH₂O and 5 μL 4×SDS-PAGE sample buffer (SB) and 10 μL of this mixture were loaded onto 10% Bis-Tris NuPAGE gels in 1×MOPS-SDS non-reduced running buffer. Gels were run at 200 V constant voltage for 50 minutes. The 3 hour and 24 hour sample gels were silver stained using a SilverXpress silver staining kit (Invitrogen) as per manufacturer's instructions.

After electrophoresis, the 48 hour samples were transferred to a nitrocellulose membrane by electroblotting at 30 V constant for 1 hour in 1× transfer buffer (NuPAGE) with 10% methanol. The membrane was probed with polyclonal antibody anti-AerA at 1/4000 dilution followed by goat anti-rabbit polyclonal antibody conjugated with alkaline phosphatase (Calbiochem) at 1/4000 dilution and developed with NBT/BCIP.

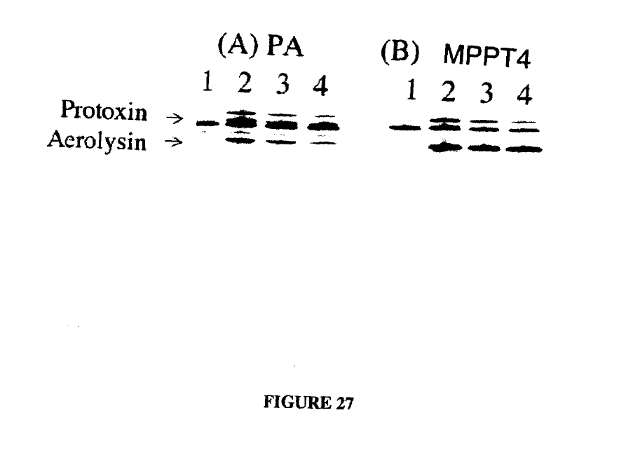

The results indicated that the silver stained gel of the 3 hour incubation samples showed little digestion for PA and complete digestion for MPPT4 (not shown). FIG. 27 shows a silver stained SDS-PAGE gel of PA and MPPT4 digested for 24 hours with MMP2 at 37° C. showing 100 ng of each sample ranging from 0-0.5 μg MMP2. The samples in each lane are: lane 1, no protease control; lane 2, 0.5 μg MMP2; lane 3, 0.25 μg MMP2; lane 4, 0.125 μg MMP2. Note that several of the bands in this figure are contributed by the MMP2 that was used. They do not represent contaminants or breakdown products in the PA and MPPT4 preparations. The silver stained gel for 24 hour incubation samples seen in FIG. 27 showed a small amount of digestion for PA and complete digestion for MPPT4 for all concentrations shown.

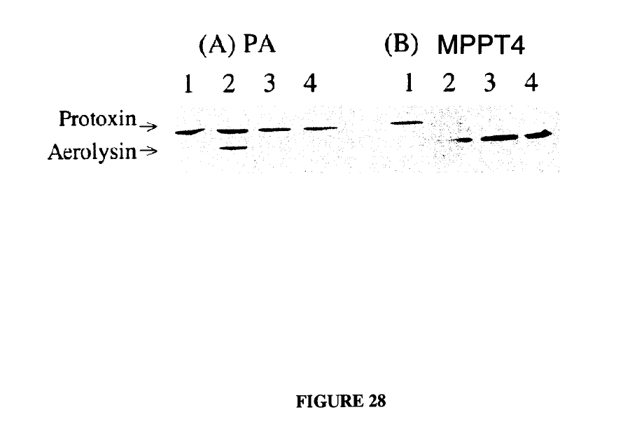

FIG. 28 shows the Western blot of 100 ng of PA and MPPT4 digested for 24 hours with 0-0.5 μg MMP2. The blot was probed with polyclonal anti-aerA antibody followed by goat anti-rabbit polyclonal antibody/alkaline phosphatase conjugate both at 1/4000 dilution and developed with NBT/BCIP. Samples in the lanes of the gel are identified as follows: lane 1, no protease control; lane 2, 0.5 μg MMP2; lane 3, 0.25 μg MMP2; lane 4, 0.125 μg MMP2. The Western blot for the 48 hour incubation samples seen in FIG. 28 shows that there is less than 50% digestion for PA and complete digestion for MPPT4.

### Example 12

### Ability of MPPT4 to Kill HT 1080 Human Fibrosarcoma Cells

A cell killing assay was performed with the MMP2 expressing cell line HT 1080. Both wild type PA and MPPT4 (toxins) were incubated with HT 1080 cells in a cell killing assay.

The HT 1080 cells were prepared to a concentration of 1×10⁶ cells/mL in fresh media. The toxins were serially diluted in 96 well plates in triplicate starting with 5 μl toxin (PA at 1 mg/mL; MPPT4 at 1.288 mg/mL) in 20 μL media. The toxins were serially diluted 1/5 across the plate. Both a negative/negative and a negative/positive control were also prepared for each plate. The cells were added to a final volume of 100 μL and incubated for 1 hour at 37° C., 5% CO₂ with humidity.

To develop the assay, 20 μL Promega cell killing media (CellTiter 96AQueous One Solution Cell Proliferation Assay) was added to each well and incubated further for 2 hours. The bubbles were popped in each well by the addition of 7 μL 70% isopropanol and the plates were measured at 490 nm using a Bio-Tek plate reader. The raw absorbance data was used to produce cell killing curves of toxin concentration vs the percentage of viable cells.

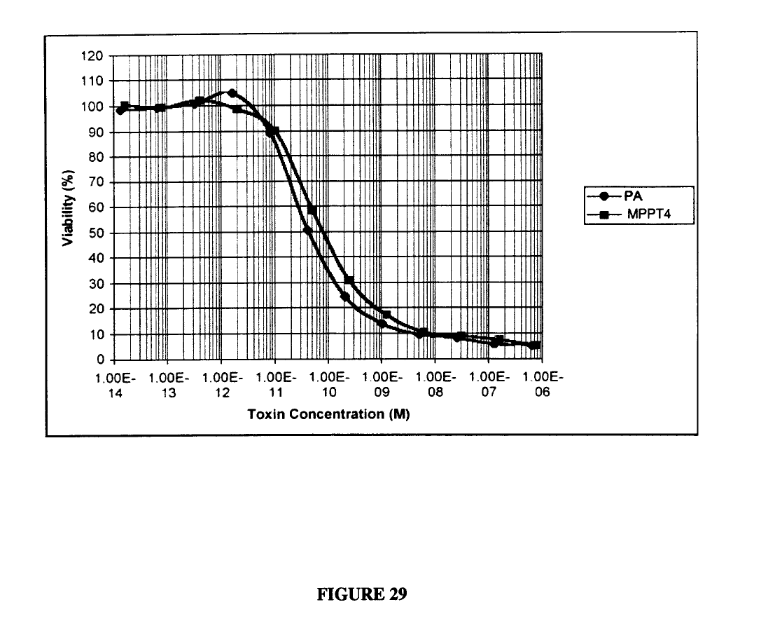

The results in FIG. 29 show that HT 1080 cells are nearly equally sensitive to PA and MPPT4 (LC₅₀s of 4×10⁻¹ M and 8×10⁻¹ M, respectively). These cells can activate PA with furin and they can activate MPPT4 with MMP2.

### Example 13

### Ability of MPPT4 to Kill EL4 Mouse Lymphoma Cells

Both wild type PA and MPPT4 (toxins) were incubated with EL4 cells (non-MMP2 expressing cells) in a cell killing assay.

The EL4 cells were prepared to 0.82×10⁶ cells/mL in fresh media. The toxins were serially diluted in 96 well plates in triplicate starting with 5 μl toxin (PA at 1 mg/mL; MPPT4 at 1.288 mg/mL) in 20 μL media. The toxins were serially diluted 1/5 across the plate. Both a negative/negative and a negative/positive control were also prepared for each plate. The cells were added to a final volume of 100 μL and incubated for 1 hour at 37° C., 5% CO₂ with humidity.

To develop the assay, 20 μL Promega cell killing media (CellTiter 96AQueous One Solution Cell Proliferation Assay) was added to each well and incubated further for 4 hours as above. The bubbles were popped in each well by the addition of 7 μL 70% isopropanol and the plates were measured at 490 nm using a Bio-Tek plate reader. The raw absorbance data was used to produce cell killing curves of toxin concentration vs the percentage of viable cells.

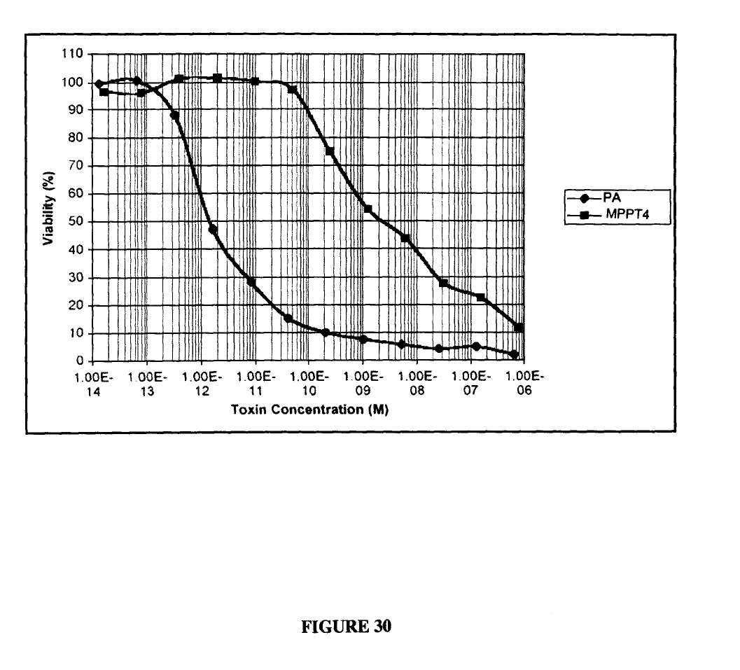

The cell killing curves for ELM cells, which do not produce MMP2, indicated that MPPT4 is much less toxic than PA (FIG. 30). This is indicated by the difference in concentration required for the toxin to kill 50% of the cells. PA requires 1.5×10⁻¹² M, whereas MPPT4 requires 2×10⁻⁹ M under the same conditions. The EL4 cells can convert PA to aerolysin using furin, however MPPT4 lacks a furin activation site.

The results depicted in Examples 11 TO 13 indicate that MPPT4 is activated by MMP2. Because the furin activation site has been removed, MPPT4 is much less toxic to normal cells. MPPT4 is as toxic as native PA to cells that express MMP2.

### Example 14

### Production of an MPPT (MPPT5) that can be Activated by Either uPA or MMP2

An MPPT derived from proaerolysin was designed that can be activated by either of two different proteases, matrix metalloproteinase 2 (MMP2) and urokinase plasminogen activator (uPA), but that cannot be activated by furin. The MPPT5 was cloned by multi-step PCR and it was purified using nickel affinity and anion exchange chromatography as described below.

Cloning of MPPT5 was carried out in two stages. The first stage was the cloning of PA-MMP2. This variant of proaerolysin with the native activation sequence (K⁴²⁷VRRAR) changed to that of MMP2 (HPVGLLAR) was prepared by three consecutive rounds of Site Directed Mutagenesis (SDM) using the Quick Change Kit (Stratagene) according to the manufacturer's instructions. In the first SDM, the plasmid pTZ18U containing the gene coding for proaerolysin with a 6×His tag at its C-terminus was used as a template, with the pair of primers MMP2-1fwd and MMP2-1rev (Table 9). The second SDM was performed using the product of the first SDM as a template and the pair of primers—MMP2-2fwd and MMP2-2rev (Table 9). The third SDM was performed using the product of the second SDM as a template and the pair of primers MMP2-3fwd and MMP2-3rev (Table 9). After the desired changes in the final product were confirmed by DNA sequencing, the mutated aerA gene was cloned (as HindIII-EcoRI fragment) into the broad host-range expression vector pMMB66HE. Recombinant clones were then transferred into Aeromonas salmonicida CB3 cells by conjugation using the filter-mating technique.

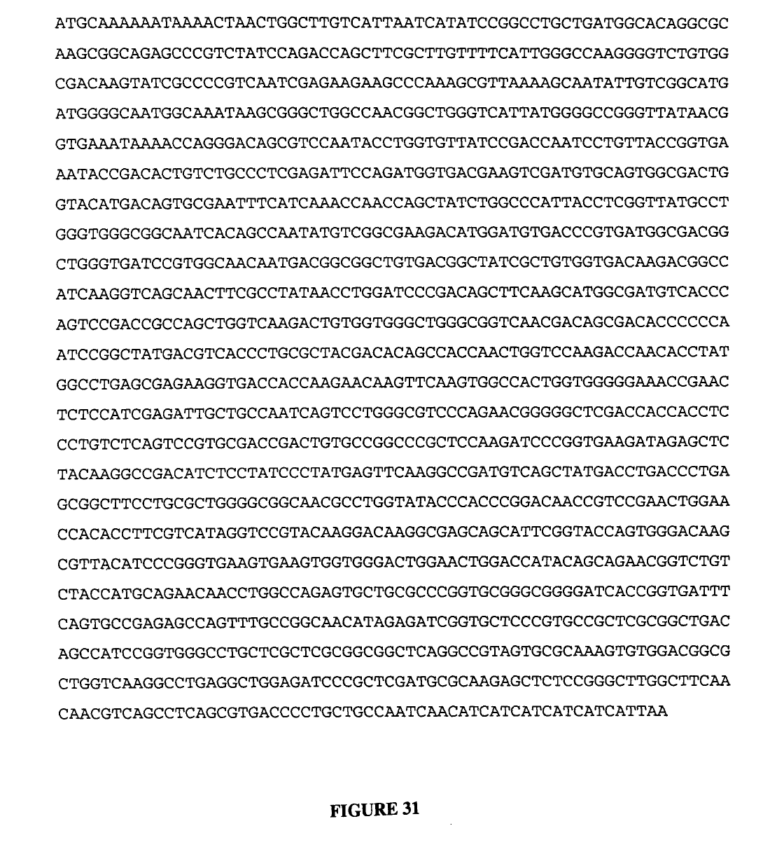

The second stage was the cloning of PA-MMP2-uPA (MPPT5). This variant of proaerolysin with the activation sequence MMP2 (HPVGLLAR) connected to that of uPA (SGRSAQ) by a GG linker was prepared by three subsequent rounds of SDM. In the first SDM plasmid pTZ18U containing the gene coding for aerolysin with the MMP2 activation sequence and a 6×His tag at its C-terminus was used as a template, with the pair of primers MMPuPA1fwd and MMPuPA1rev (Table 9). The second SDM was performed using the product of the first SDM as a template and the pair of primers—MMPuPA2fwd and MMPuPA2rev (Table 9). The third SDM was performed using the product of the second SDM as a template and the pair of primers MMPuPA3fwd and MMPuPA3rev (Table 9). After the desired changes in the final product were confirmed by DNA sequencing, the mutated aerA gene was cloned (as HindIII-EcoRI fragments) into broad host-range expression vector pMMB66HE. Recombinant clones were then transferred into Aeromonas salmonicida CB3 cells by conjugation using the filter-mating technique. The nucleotide sequence of MPPT5 (SEQ ID NO:40) is shown in FIG. 31.

TABLE 9
Primers used in Quick Change reactions

| | | SEQ ID |
|---|---|---|
| Primer | Sequence | NO |
| MMP2-1fwd | CTCGCGGCTGACAGCCATCCGGTGCGTGCTCG | 26 |
| | CAG | |
| MMP2-1rev | CTGCGAGCACGCACCGGATGGCTGTCAGCCGC | 27 |
| | GAG | |
| MMP2-2fwd | GACAGCCATCCGGTGGGCCTGCTCAGTGTGGA | 28 |
| | CGGC | |
| MMP2-2rev | GCCGTCCACACTGAGCAGGCCCACCGGATGGC | 29 |
| | TGTC | |
| MMP2-3fwd | CCGGTGGGCCTGGTCGCTCGCAGTGTGGACGG | 30 |
| | C | |
| MMP2-3rev | GCCGTCCACACTGCGAGCGAGCAGGCCCACCG | 31 |
| | G | |
| MMPuPA1fwd | GGCCTGCTCGCTCGCGGCGGCTCAAGTGTGGA | 32 |
| | CGGC | |
| MMPuPA1rev | GCCGTCCACACTTGAGCCGCCGCGAGCGAGCA | 33 |
| | GGCC | |
| MMPuPA2fwd | GCTCGCGGCGGCTCAGGCCGTAGTGTGGAGGG | 34 |
| | C | |
| MMPuPA2rev | GCCGTCCACACTACGGCCTGAGCCGCCGCGAG | 35 |
| | C | |
| MMPuPA3fwd | GGCTCAGGCCGTAGTGCGCAAAGTGTGGACGG | 36 |
| | C | |
| MMPuPA3rev | GCCGTCCACACTTTGCGCACTACGGCCTGAGC | 37 |
| | C | |

### Expression and Purification of MPPT5

A single colony of CB3MPPT5::pMMB66 (CB3 strain containing a plasmid expressing MPPT5) was inoculated into 30 mL of LB media (10% Bacto Tryptone, 5% Bacto Yeast Extract, 10% NaCl, pH 7.5) containing Davis minimal media, 0.2% glucose, 100 μg/mL ampicillin and 40 μg/mL of both kanamycin and rifampicin. The culture was grown at 27° C. with shaking at 250 rpm overnight to an OD_{600 nm} of 4.62. The culture was subinoculated (1%) into 5×2 L flasks containing a final volume of 500 mL of the above media excluding rifampicin. Cultures were grown as above for 5.5 hours to OD_{600 nm} from 0.95-0.99 and induced with a final concentration of 1 mM IPTG. The induced cultures were incubated as above for 18 hours. The culture supernatant was harvested by pelleting the cells at 10,000 rpm in a JA16.25 rotor for 15 minutes at 4° C. A few drops of polypropylene glycol were added to the supernatant to prevent frothing during concentration with a KVICK SCU concentrator (Amersham, 10,000 kDa cutoff). The supernatant was concentrated from 2.5 L to 100 mL and aliquoted in 25 mL fractions for storage at −20° C.

An aliquot of MPPT5 supernatant was thawed and clarified by centrifugation in a JA25.5 rotor at 10,000 rpm for 10 minutes at 4° C. The clarified supernatant was loaded at 1 mL/min onto a 10 mL FPLC nickel affinity column (Amersham Chelating Sepharose Fast Flow resin) equilibrated in Buffer A (20 mM Na₂HPO₄, 0.5M NaCl, 10 mM imidazole, pH 7.4). The column was washed at 5 mL/min with 50 mL of Buffer A and eluted with 60% Buffer B (20 mM Na₂HPO₄, 0.5M NaCl, 0.5M imidazole, pH 7.4) at approximately 300 mM imidazole. The toxin containing fractions were desalted into 20 mM Tris, 1 mM EDTA, 150 mM NaCl, pH 7.4 using PD-10 exchange columns (Amersham). The desalted material was loaded at 1 mL/min onto an FPLC HiPrep 16/10 Q FF anion exchange column (Amersham) equilibrated in the same buffer as used for desalting. The column was washed in equilibration buffer and eluted with a linear gradient of 0.15-1 M NaCl in 20 mM Tris, 1 mM EDTA, pH 7.4. Concentrations were determined by measuring absorbance at 280 nm.

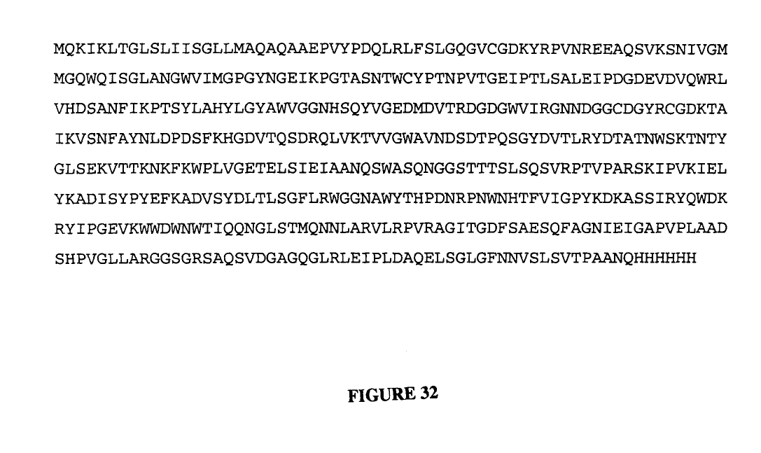

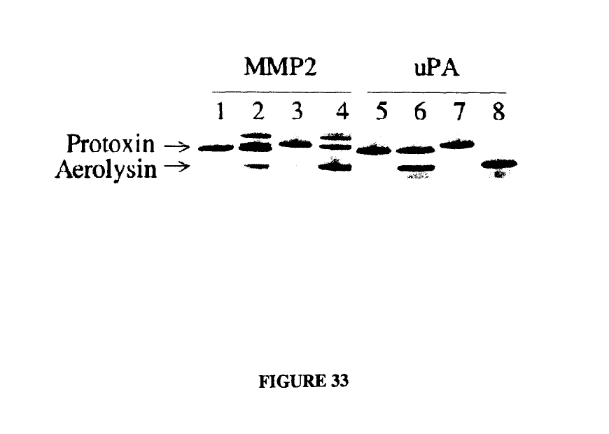

This purification procedure yielded pure protein as determined by silver stained SDS-PAGE (FIG. 33). The amino acid sequence of MPPT5 (SEQ ID NO:41) is depicted in FIG. 32.

### Example 15

### Activation of MPPT5 by MMP2 and uPA

The sensitivity of MPPT5 to MMP2 and uPA was demonstrated using silver stained SDS-PAGE and a hemolytic titer assay.

Digestion of MPPT5 with MMP2. PA and MPPT5 were prepared in HBS, 10 mM CaCl₂, pH 7.4 to a concentration of 0.06 mg/mL with or without the addition of 0.5 μg of MMP2 in a total of 60 μL. The reaction mixtures were incubated for 2 hours at 37° C. and then inhibited with a final concentration of 1 μM 1,10-o-phenanthroline followed by chilling on ice.

Hemolytic titer. This assay was carried out as described in Example 3. The titer value, number of wells containing lysed cells, was estimated visually after incubation at 37° C. for 1 hour.

Silver stained SDS-PAGE. For each reaction mixture, 100 ng of material was prepared in 1× sample buffer and run on a 10% Bis-Tris NuPAGE gel in 1×MOPS-SDS running buffer under non-reduced conditions for 50 minutes at 200 V constant voltage. The gel was silver stained using a SilverXpress silver staining kit (Invitrogen) following the manufacturer's instructions.

FIG. 33 depicts a silver stained gel of PA and MPPT5 digested with MMP2 or uPA for 2 hours at 37° C. Each lane contains 100 ng of material identified as follows: lane 1, PA control; lane 2, PA with MMP2; lane 3, MPPT5 control; lane 4, MPPT5 with MMP2; lane 5, PA control; lane 6, PA with uPA; lane 7, MPPT5 control; lane 8, MPPT5 with uPA.

The results shown in FIG. 33 indicate that MPPT5 is sensitive to digestion by both MMP2 and uPA. PA is not measurably affected by MMP2, but it is somewhat sensitive to uPA, though not to the degree seen for MPPT5. The aerolysin product of the uPA digestion is slightly larger than that of the MMP2 product. This indicates that the uPA cuts only at the uPA site, which follows the MMP2 site after a 2 amino acid linker, and that uPA does not cut at the MMP2 site.

The corresponding hemolytic titer data is displayed in Table 10. Titers were read after 60 minutes incubation at 37° C. The data confirm that PA was not activated by MMP2, and only partially activated by uPA, whereas MPPT5 was activated by both proteases. PA and MPPT5 were digested with MMP2 and uPA for 120 minutes at 37° C.

TABLE 10
Hemolytic titers of proaerolysin and MPPT5

Control in HBS,

| | 10 mM CaCl₂ | Control in HBS | MMP2 | uPA |
|---|---|---|---|---|
| PA | 0 | 0 | 0 | 5 |
| MPPT5 | 0 | 0 | 6.5 | 7 |

### Example 16

### Ability of MPPT5 to Kill HT 1080 Human Fibrosarcoma Cells

The ability of MPPT5 to kill MMP2 expressing cells was tested as follows. Both wild type PA and MPPT5 were incubated with HT 1080 cells (MMP2 expressing) in a cell killing assay. The HT 1080 cells were prepared to a concentration of 1×10⁶ cells/mL in fresh media. The proteins were serially diluted in 96 well plates in triplicate starting with 5 μl toxin (PA at 1 mg/mL; MPPT5 at 1.46 mg/mL) in 20 μL media. The toxins were serially diluted 1/5 across the plate. Both a negative/negative and a negative/positive control were also prepared. The cells were added to a final volume of 100 μL and incubated for 1 hour at 37° C., 5% CO₂ with humidity. To develop the assay, 20 μL Promega cell killing media (CellTiter 96AQueous One Solution Cell Proliferation Assay) was added to each well and incubated further for 2 hours as above. The bubbles were popped in each well by the addition of 7 μL 70% isopropanol and the plates were measured at 490 nm using a Bio-Tek plate reader. The raw absorbance data was used to produce cell killing curves of toxin concentration vs the percentage of viable cells.

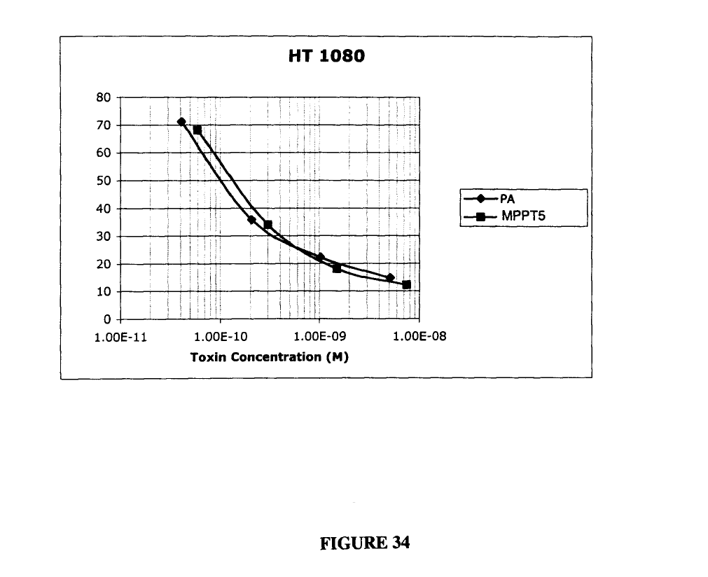

The cell killing data for HT 1080 (MMP2 expressing) cells, as shown in FIG. 34, indicates that HT 1080 cells were nearly equally sensitive to PA and PA-MMP2 (LC50's of 1×10⁻¹⁰ M and 1.5×10⁻¹⁰ M, respectively). These cells were able to activate PA with furin and they could activate MPPT5 with MMP2.

### Example 17

### Ability of MPPT5 to Kill Hela Human Cervical Cancer Cells or A2058 Human Melanoma Cells

The ability of MPPT5 to kill urokinase receptor expressing cells, with and without the addition of pro-uPA and glu-plasminogen was tested as follows. The urokinase receptor expressing cell lines used were HeLa cells and A2058 cells. Two 96 well Costar titer plates for each adherent cell line were prepared containing either HeLa or A2058 cells at an estimated 50% confluency. Since A2058 cells grow faster than HeLa cells, it was likely that there were more cells in the A2058 plates. The plates were washed twice in warmed plain DMEM media by pipetting off old media and replacing with 100 μl new media. The cells were preincubated with or without 0.1 μg/mL pro-urokinase and 1 μg/mL glu-plasminogen in a final volume of 240 μl media for 30 minutes at 37° C., 5% CO₂ with humidity. Either PA or MPPT5 (1 mg/mL and 1.46 mg/mL, respectively) was added to the first well in each row excluding those used for negative/negative and positive/negative controls, which received media to bring the volumes to 250 μL. The wells were serially diluted 1:5 in media with or without pro-urokinase and glu-plasminogen across the titer plate (200 μL remaining in each well). The plates were incubated for 1 hour at 37° C., 5% CO₂ with humidity.

To develop the plates, the old media was removed and 200 μL of fresh media containing FBS was added back in addition to 50 μL of 2.5 mg/mL MTT. The plates were incubated as above for 80 minutes until purple precipitate became visible at the bottom of the wells.

The media was carefully removed without disturbing the precipitate and 100 μL of solubilization buffer (0.5% (w/v) SDS and 25 mM HCl in 90% (v/v) isopropanol) was added to each well. The openings of the wells were sealed with aluminum sealing foil and the plates were gently agitated at room temperature to solubilize the precipitate. Plates were stored overnight at 4° C.

The plates were measured at 560 nm using a 96 well plate reader and the raw data was used to produce cell killing curves of protein concentration vs cell viability.

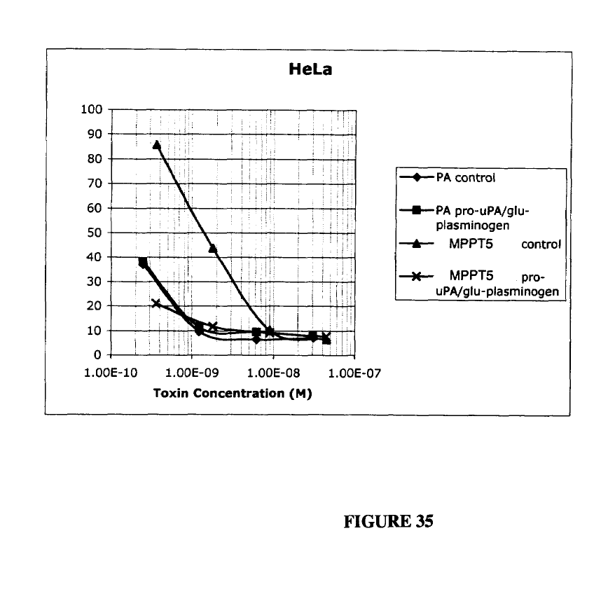

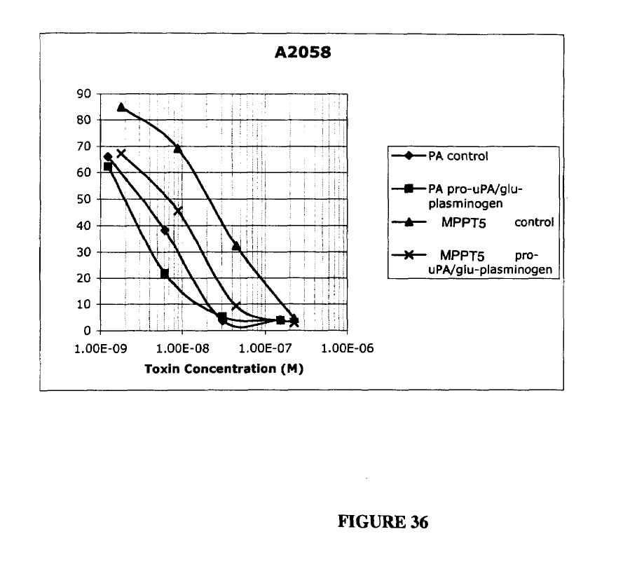

The cell killing curves for HeLa and A2058 (uPAR expressing) cells, as shown in FIGS. 35 and 36, respectively, indicate that both cell lines are sensitive to PA and MPPT5, although cellular toxicity of the MPPT5 toxin is significantly increased by the addition of both pro-uPA and glu-plasminogen. This indicates that both HeLa and A2058 cells are capable of facilitating the production of active uPA via interactions at uPAR expressed on the cell surface. The toxicity of PA was not affected by the addition of pro-uPA and glu-plasminogen.

### Example 18

### Ability of MPPT5 to Kill Mouse Lymphoma EL4 Cells

The ability of MPPT5 to kill cells that do not express urokinase receptor, with and without the addition of pro-uPA and glu-plasminogen was determined as follows. Both wild type PA and MPPT5 (toxins) were incubated with EL4 cells in a cell killing assay with or without the addition of pro-uPA/glu-plasminogen. Two batches of EL4 cells were prepared to a concentration of 1×10⁶ cells/mL in fresh media. To one batch, pro-uPA and glu-plasminogen were added to 0.1 μg/mL and 1 μg/mL, respectively. Both preparations were incubated for 30 minutes at 37° C., 5% CO₂ with humidity.

The toxins were serially diluted in 96 well plates in triplicate starting with 5 μl toxin (PA at 1 mg/mL; MPPT5 at 1.46 mg/mL) in 20 μL media. The toxins were serially diluted 1/5 across the plate. Both a negative/negative and a negative/positive control were also prepared. The cells were added to a final volume of 100 μL and incubated for 1 hour at 37° C., 5% CO₂ with humidity.

To develop the assay, 20 μL Promega cell killing media (CellTiter 96AQueous One Solution Cell Proliferation Assay) was added to each well and incubated further for 4 hours as above. The bubbles were popped in each well by the addition of 7 μL 70% isopropanol and the plates were measured at 490 nm using a Bio-Tek plate reader. The raw absorbance data was used to produce cell killing curves of toxin concentration vs the percentage of viable cells.

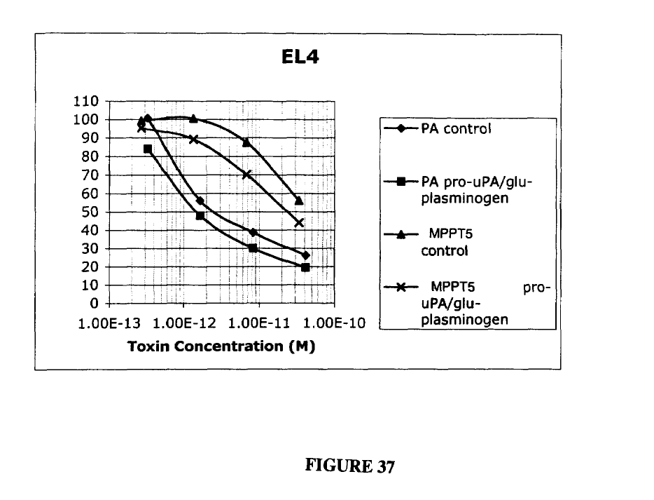

The cell killing curves for EL4 (non-MMP2 expressing and non-uPAR expressing) cells, shown in FIG. 37, indicate that EL4 cells are less sensitive to MPPT5 than to PA. The EL4 cells can convert native PA to aerolysin using furin, however MPPT5 lacks a furin activation site. The addition of pro-uPA and glu-plasminogen did not significantly increase the toxicity of either protein, as EL4 cells do not express uPAR, which is required for the production of active uPA.

The data shown in Examples 15 to 18 indicate that MPPT5 can be activated by either MMP2 or uPA. Because the furin activation site has been removed, MPPT5 is much less toxic to normal cells that do not express either MMP2 or uPAR. MPPT5 is as toxic as native PA to cells that express MMP2, and is toxic to uPAR expressing cells when pro-uPA and glu-plasminogen are present.

Examples 19 to 24 presented below relate to modified forms of proaerolysin (PA) that contain an ARD and/or a large binding domain mutation. The activation sequence of any one of these modified PA proteins could be modified to provide a MPPT in accordance with the present invention using standard techniques such as those described herein.

### Example 19

### Production of Proaerolysin with AFAI as a Targeting Unit and an R336A Mutation (AFA-PA-R336A)

A modified PA protein comprising an AFAI as a targeting unit and a mutation in a native binding domain (AFA-PA-R336A) was prepared as follows. This protein also included a histidine tag. The AFA-PA-R336A protein was prepared using AFA-PA-his::pTZ18U (or AFA-PA-H6::pTZ18U preparation described in Example 2) as a starting point. In order to change amino acid R336 to an alanine (R336A), two primers were synthesized:

R336A fwd:

(SEQ ID NO:55)

5′-CAC CCG GAC AAC GCA CCG AAC TGG AAC-3′
R336A rev:

(SEQ ID NO:56)

5′-GTT CCA GTT CGG TGC GTT GTC CGG GTG-3′

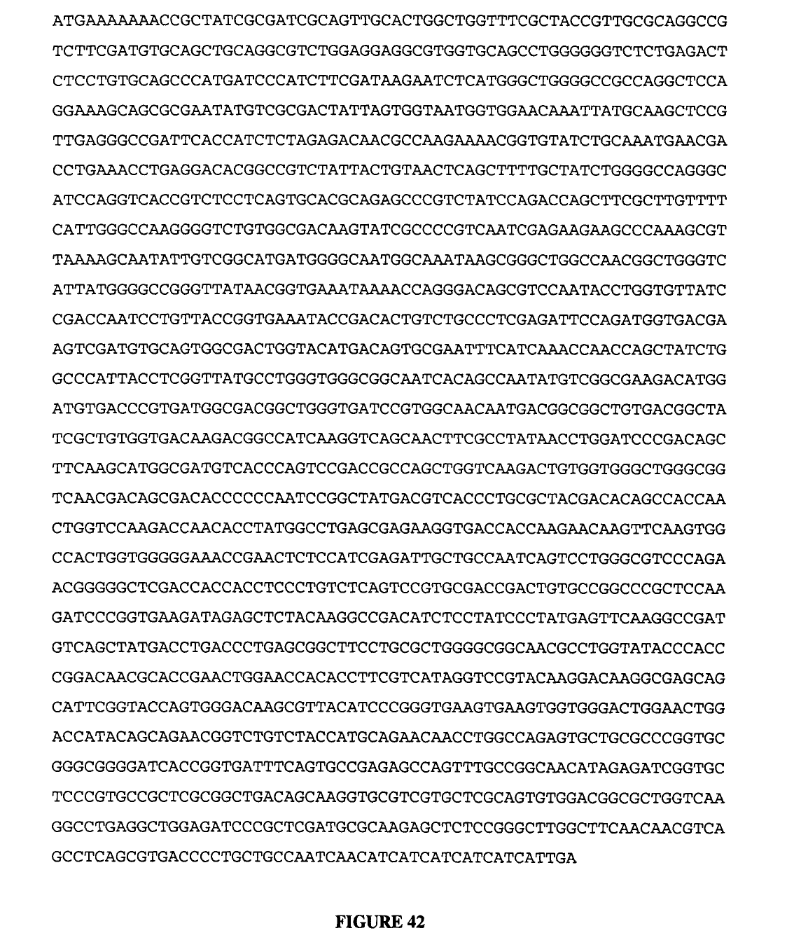

These two primers, along with plasmid AFA-PA-H6::pTZ18U as the template, were used to create the R336A mutation using the Quickchange II Site-Directed Mutagenesis kit (Stratagene) as per the manufacturer's instructions. The resulting clones were sequenced to ensure the correct mutation was made. The resulting plasmid was called AFA-PA(R336A)-H6::pTZ18U and the nucleotide sequence of AFA-PA-R336A (SEQ ID NO:44) is shown in FIG. 42.

In order to express AFA-PA(R336A)-H6 proteins, the coding region was cut out of AFA(R336A)-H6::pTZ18U with EcoRI and HindIII, and cloned into the EcoRI and HindIII sites of the wide-host expression vector pMMB67EH. The resulting plasmid AFA-PA(R336A)-H6::pMMB67EH was conjugated into CB3 using the filter mating technique to produce the strain CB3 AFA-PA(R336A)-H6::pMMB67EH.

A single colony of CB3 AFA1-PA R336A-His6::pMMB67EH was picked and inoculated into 30 ml of LB Davis media (1% Bacto Tryptone [BBL], 0.5% Yeast Extract [BBL], 1% NaCl) containing 0.2% glucose, 40 μg/ml rifampicin, 40 μg/ml kanamycin and 100 μg/ml ampicillin. The culture was grown overnight at 27° C. with shaking at 250 rpm. Five 2 L flasks, each containing 500 ml of media as above, were subinoculated (1%) with overnight culture (A_{600 nm} 3.70) and incubated at 27° C. with shaking at 250 rpm. When the A_{600 nm} of the cultures reached 0.64-0.72, IPTG was added to a final concentration of 1 mM, to induce AFA-PA R336A-His production. The flasks were incubated at 27° C. with shaking at 250 rpm for 17.25 hours. When the overnight A_{600 nm} of the cultures reached 2.6-3.2, the contents of the flasks were pooled and the culture was centrifuged at 10,000 rpm in a JA-16.25 rotor (Beckman) for 15 minutes at 4° C. The culture supernatant was collected and 6 drops of polypropylene glycol were added to prevent frothing during concentration. The protease inhibitor 1,10-o-phenanthroline was added to a final concentration of 1 mM.

The culture supernatant was concentrated from 2.4 L to ˜50 ml using a Kvick Lab SCU 10,000 Dalton cutoff membrane (Amersham; polyethersulfone membrane with polypropylene screen and a 0.11 m² filtration area) on ice. PMSF was added to a final concentration of 1 mM.

The concentrate was centrifuged at 10,000 rpm using a JA-25.5 (Beckman) rotor for 10 minutes at 4° C. to clarify. At 4° C., the supernatant was pooled (50 ml) and loaded at 1 ml/min onto a 5 ml XK16 Ni²⁺ column (Chelating Sepharose Fast Flow, Amersham) equilibrated in 20 mM Na₂HPO₄, 0.5 M NaCl, 10 mM imidazole, pH 7.4. The column was washed at 2.5 ml/min with 100 ml equilibration buffer (above) to remove any unbound material. The column was eluted for 50 ml with 20 mM Na₂HPO₄, 0.5 M NaCl, 200 mM imidazole, pH 7.4 at 2.5 ml/min. The protein containing fractions were loaded onto a PD10 column (Amersham) equilibrated in 20 mM HEPES, 0.1 M NaCl, 1 mM EDTA, pH 7.4 in 2.5 ml aliquots and eluted with 3.5 ml of the same buffer. The PD10 fractions were pooled and loaded at 0.25 ml/min onto a DEAE column (75 ml XK16, CL-6B DEAE, Amersham) equilibrated in the same buffer. The column was eluted with a 0.1-0.4 M NaCl linear gradient in 20 mM HEPES, 1 mM EDTA, pH 7.4 at 0.25 ml/min. The protein containing fractions were left in the elution buffer as the final storage buffer. The concentrations were determined by measuring A_{280 nm} values.

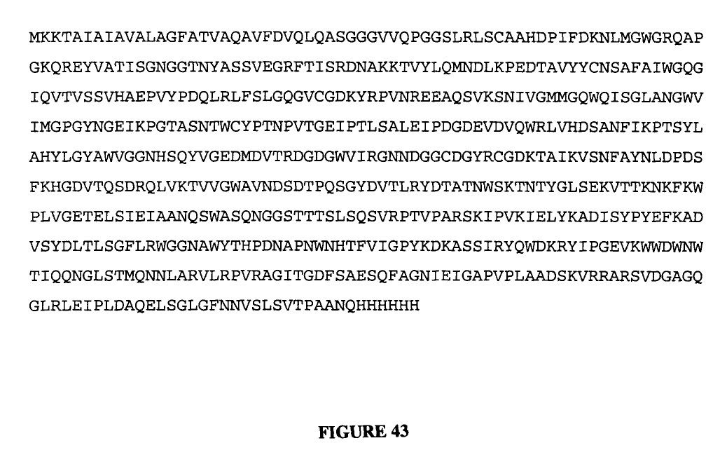

The amino acid sequence of AFA-PA-R336A (SEQ ID NO:45) is shown in FIG. 43.

### Example 20

### Production of Proaerolysin with an R336a Mutation (PA-R336A)

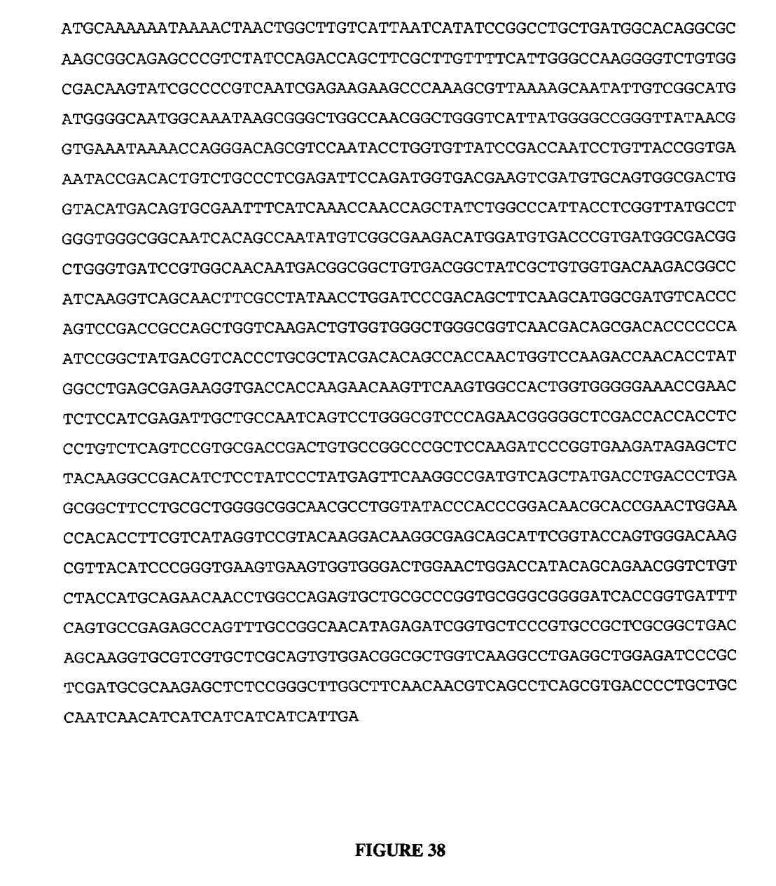

The plasmid AFA-PA(R336A)-H6::pTZ18U, prepared as described in Example 19, was then used to create a proaerolysin construct containing the R336A mutation. AFA-PA(R336A)-H6::pTZ18U was digested with XhoI and StuI to give a 1046 nt fragment that contains the portion of aerA with the R336A change. This was cloned into the XhoI and StuI restriction sites of PA-H6::pTZ18U to give a complete aerA gene containing the R336A change. The resulting clone was checked by sequencing and the plasmid was called PA(R336A)-H6::pTZ18U. The nucleotide sequence of PA-R336A (SEQ ID NO:42) is shown in FIG. 38.

In order to express PA-R336A protein, the coding region was cut out of PA-H6::pTZ18U with EcoRI and HindIII, and cloned into the EcoRI and HindIII sites of the wide-host expression vector pMMB66HE. The resulting plasmid PA(R336A)-H6::pMMB66HE was conjugated into CB3 using the filter mating technique to produce the strain CB3 PA(R336A)-H6::pMMB66HE.

A single colony of CB3-PA-R336A-His6::pMMB66 was grown overnight at 27° C. with shaking at 250 rpm in 30 ml Phytone Davis media (1% Phytone Peptone [BBL], 0.5% Yeast Extract [BBL], 1% NaCl) containing 0.2% glucose, 100 μg/ml ampicillin, 40 μg/ml kanamycin and 40 μg/ml rifampicin. Five 2 L flasks, each containing Phytone Davis media as above with ampicillin and kanamycin only, were subinoculated (1%) with overnight culture (A_{600 nm} 3.75) and incubated at 27° C. with shaking at 250 rpm. When the A_{600 nm} of the cultures reached 1.1-1.2, IPTG was added to a final concentration of 1 mM, to induce toxin production. The cultures were incubated at 27° C. with shaking at 250 rpm for 16 hours. When the A_{600 nm} of the cultures reached 4.6-5.2, the contents of the flasks were pooled and the culture was centrifuged at 10,000 rpm using a JA-16.25 (Beckman) rotor for 15 minutes at 4° C. The culture supernatant was saved and 6 drops of polypropylene glycol were added to prevent frothing. The protease inhibitor 1,10-o-phenanthroline was added to a final concentration of 1 mM.

The material was concentrated from 2.5 L to 45 ml using a Kvick Lab SCU 10,000 Dalton cutoff membrane (Amersham; polyethersulfone membrane with polypropylene screen and a 0.11 m² filtration area) on ice. PMSF was added to a final concentration of 1 mM. The material was aliquoted and frozen at −20° C. for future use.

An aliquot of the concentrated material was thawed after 1 day of storage at −20° C. and centrifuged at 10,000 rpm using a JA-25.5 (Beckman) rotor for 10 minutes at 4° C. to clarify. The clarified material was loaded onto a 10 ml XK16 FPLC Ni²⁺ (Amersham Chelating Sepharose Fast Flow Resin) column equilibriated in 20 mM Na₂HPO₄, 0.5 M NaCl, 10 mM imidazole, pH 7.4 at 1 ml/min. The column was washed for 50 ml at 5 ml/min with equilibration buffer to remove unbound material and eluted at a 59.2% step gradient of 500 mM imidazole in 20 mM Na₂HPO₄, 0.5 M NaCl, 10 mM imidazole pH 7.4 for 40 ml giving a final imidazole concentration of 300 mM. The elution fractions were collected in 4 ml aliquots. The peak fractions were pooled and loaded onto a 150 ml XK26 FPLC Sepharose G25 (Sigma) column equilibrated in 20 mM Tris, 1 mM EDTA, 0.15 M NaCl, pH 7.4 at 1 ml/min and eluted with equilibration buffer at 2 ml/min. Peak fractions were collected in 4 ml aliquots and pooled yielding 28 ml of material. The desalted material was loaded onto an FPLC HiPrep 16/10 Q FF (Amersham) column equilibrated in desalting buffer at 1 ml/min. The column was eluted for 60 ml with a 0.15-1 M NaCl gradient in 20 mM Tris, 1 mM EDTA, pH 7.4 at 2 ml/min. The peak fractions were collected in 4 ml aliquots and the concentrations determined by measuring A_{280 nm} values.

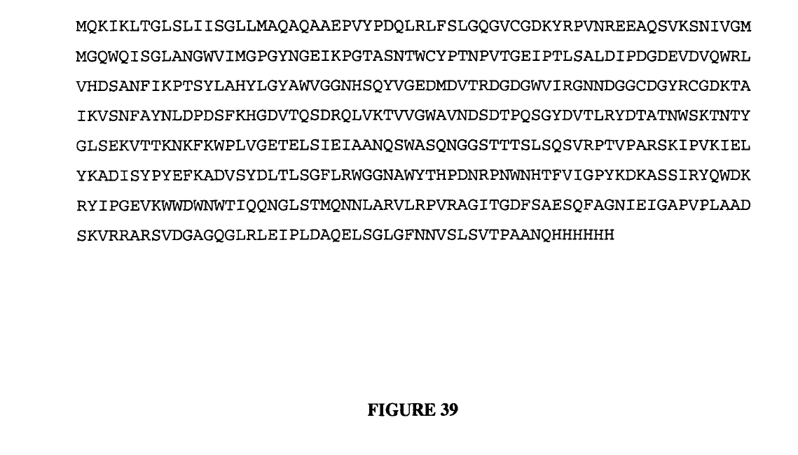

The amino acid sequence of PA-R336A (SEQ ID NO:43) is shown in FIG. 39.

### Example 21

### Production of Proaerolysin with an R336C Mutation (PA-R336C)

The codon for arginine 336 was changed to a cysteine codon using the Quickchange mutagenesis kit (Stratagene). Two primers were synthesized as follows:

(SEQ ID NO:60)

Forward 5′-CACCCGGACAACTGTCCGAACTGGAACCACAC-3′

(SEQ ID NO:61)

Reverse 5′-GGTTCCAGTTCGGACAGTTGTCCGGGTGGGTAAA-3′

The plasmid PA::pTZ18U was used as the template in the mutagenesis reaction, and the PCR product was digested with EcoRI and HindIII and ligated into the EcoRI and HindIII sites of the vector pMMB66HE. This plasmid (PA-R336c::pMMB66HE) was transconjugated into CB3 as described above, and the strain CB3 PA-R336c::pMMB66HE was used to purify the protein PA-R336c.

Purification of PA-R336c

CB3 PA-R336c::pMMB66EH was inoculated into 25 ml of LB media (1% Bacto Tryptone [BBL], 0.5% Yeast Extract [BBL], 1% NaCl) containing 1× Davis buffer (Miller, 1972; LB-Davis media), 0.2% glucose, 40 μg/ml rifampicin, 40 μg/ml kanamycin and 100 μg/ml ampicillin and grown overnight at 27° C./250 rpm. It was then subinoculated (1%) into the same medium and incubated at 27° C./250 rpm. When the culture reached an OD_{600 nm} of approx. 0.7, IPTG was added to a final concentration of 1 mM to induce PA-R336C production and incubation was continued for 17.5 hours. The overnight cultures (OD_{600 nm}=5.8-6.0) were centrifuged in a JA-16.25 rotor (Beckman) at 10,000 rpm/15 minutes/4° C. The culture supernatant was collected and concentrated from 2.4 L to ˜70 ml using a Sartorius Sartocon Mini-concentrator with a 20,000 Dalton cutoff membrane (cellulose triacetate membrane/0.7 m² filtration area) at 4° C.

The concentrate was centrifuged at 10,000 rpm using a JA-25.5 (Beckman) rotor for 10 minutes. The supernatant was loaded onto a desalting column (150 ml XK26 Sephadex G25 coarse, Sigma) equilibrated in 20 mM NaH₂PO₄, 0.3 M NaCl, pH 6.0, and eluted with the same buffer at 1 ml/min. The protein-containing fractions were identified by measuring A₂₈₀ values of the fractions, and the peak tubes were loaded onto a hydroxyapatite column (40 ml XK16, BioRad) equilibrated in the same buffer. The column was eluted with a 20-150 mM NaH₂PO₄ linear gradient in 0.3 M NaCl, pH 6.0 at 0.25 ml/min. Protein containing fractions were pooled and precipitated with 60% ammonium sulfate for 2 hours on ice. The precipitated material was pelleted by centrifugation at 15,000 rpm for 10 minutes at 4° C. using a JA-25.5 rotor (Beckman). The pellet was resuspended in 15 ml of 20 mM HEPES, 1 mM EDTA, 5 mM β-mercaptoethanol, pH 7.4, and then loaded onto a DEAE column (75 ml XK16 CL-6B DEAE, Amersham) equilibrated in the same buffer. The column was eluted with a 0.1-0.4 M NaCl gradient in 20 mM HEPES, 1 mM EDTA, 5 mM β-mercaptoethanol, pH 7.4, collecting fractions of approximately 5.3 ml.

### Example 22

### Ability of the Modified Proaerolysin PA-R336A to Bind to Mouse Lymphoma EL4 Cells

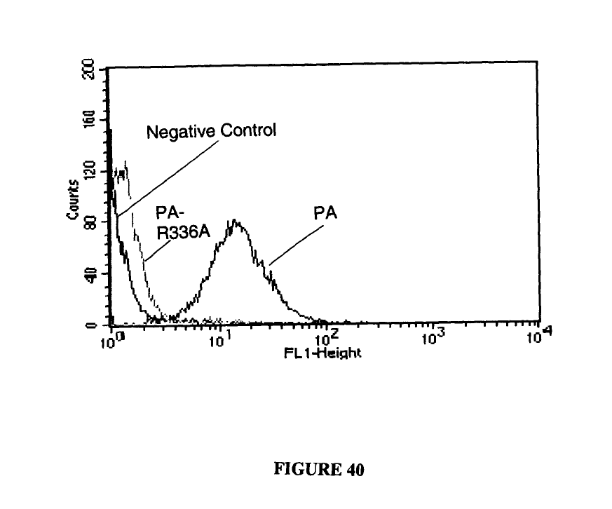

The ability of the modified proaerolysin with a mutation in the large lobe binding domain (PA-R336A) to bind to EMA mouse lymphoma cells was determined by flow cytometry using a FacsCalibur Flow Cytometer (Pearson). EL4 cells were prepared in unsupplemented cell culture media to a concentration of 2×10⁶ cells/mL by resuspending the cell pellet after centrifugation at 1100 rpm for 5 minutes at room temperature in an IEC Centra CL2 centrifuge. PA or PA-R336A was added to cells at 2×10⁻⁸ M and incubated on ice for 30 minutes. Cells were centrifuged for 7 seconds in an IEC Microlite microfuge, washed 3× in unsupplemented media and resuspended to the original volume. Rabbit anti-aerolysin polyclonal antiserum was added to the cells at a 1/500 dilution and the mixture was incubated on ice for 30 minutes. Cells were washed and resuspended as above. The secondary antibody, a polyclonal goat α-rabbit IgG H+ L chain specific Fluorscein conjugate (CalBiochem), was added at a 1/5000 dilution and incubated on ice for 30 minutes. Cells were washed and resuspended as above. A histogram was prepared using CellQuest software and is shown in FIG. 40. The results as shown in FIG. 40 demonstrates that, as expected, the R336A mutation in the native binding domain of proaerolysin decreased the ability of PA-R336A to bind to EL4 cells.

### Example 23

### Ability of the Modified Proaerolysin PA-R336A to Kill Mouse Lymphoma EL4 Cells

The ability of PA-R336A to kill cells was determined as follows. Cell killing assays were performed using EL4 (mouse) cells prepared at a concentration of 1×10⁶ cells/mL by resuspending cell pellets in cell culture media (DMEM with 10% FBS) after centrifugation at 1100 rpm for 5 minutes at room temperature in an IEC Centra CL2 centrifuge. Each assay was carried out in triplicate. Using 96 well titer plates, the toxins PA and PA-R336A were serially diluted 1:5 in supplemented cell media using a final volume of 20 μL. After serial dilution, cells were added to appropriate rows to a volume of 100 μL. Two control rows were used: the first containing no toxin and no cells and the second containing no toxin with cells. The cells were then preincubated with the toxins for 1 hour at 37° C., 5% CO₂ with humidity before adding 20 μL of cell killing reagent (Promega CellTiter 96 AQueous One Solution Cell Proliferation Assay) with further incubation for 4 hours under the same conditions. After incubation, 7 μL of 70% isopropyl alcohol was added to each well in order to pop air bubbles. The plates were read using a BioTec plate reader at 490 nm. Cell killing curves were generated by plotting the calculated average percent cell viability for each well against the respective toxin concentration.

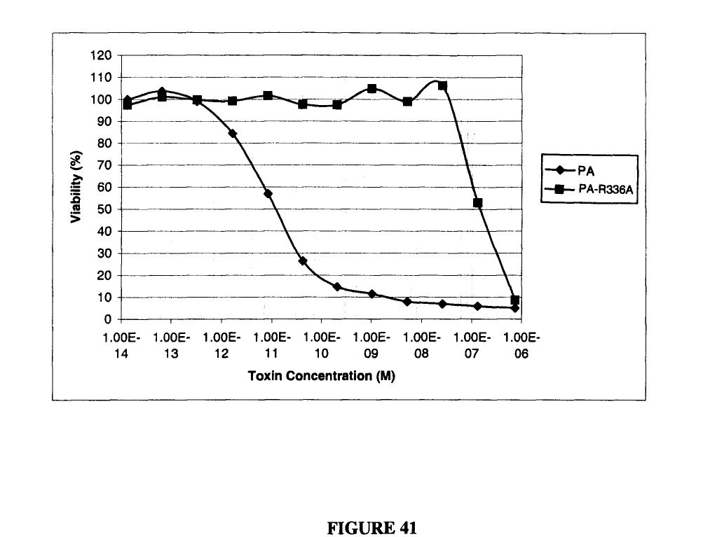

The results of this assay are shown in FIG. 41, and indicate that, as expected from the decreased binding ability of PA-R336A shown in Example 22, the ability of this protein to kill cells was decreased compared to that of PA.

### Example 24

### Ability of the Modified Proaerolysins AFA-PA R336A and PA-R336C to Kill Mouse Lymphoma EL4 and Human Lung Cancer A549 Cells

The effect of the R336A mutation on the ability of AFA-PA-R336A to kill mouse lympohoma EL4 and human lung cancer A549 cells was tested and compared to that of toxins with the R336C mutation (PA-R336C), and to a control toxin containing AFAI as an artificial regulatory domain but no binding domain mutation (AFA-PA). Cell killing assays in both types of cells were carried out in a manner similar to that described in Examples 13 and 21.

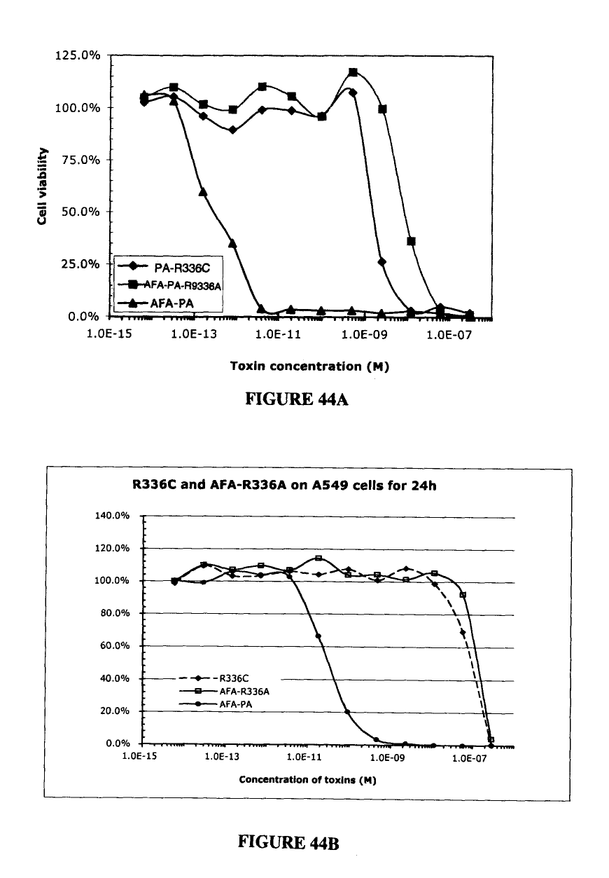

The results are shown in FIG. 44A (EL4 cells) and B (A549 cells) and indicate that the toxicity of AFA-PA-R336A and PA-R336c to both types of cells was reduced by the substitution of arginine in the binding domain. The results also indicated that the AFAI moiety acts as an inhibitor of the modified proaerolysin in that the AFA-PA-R336A protein did not show an improvement in activity over the PA-R336C protein in lung cancer cells, which carry the target for the AFAI moiety. This suggests that the addition of the AFAI moiety with a linker that could be cleaved, for example, by an enzyme associated with cancer cells would be a valuable approach for targeting and activating the modified proaerolysin to cancer cells, while decreasing the toxicity of the modified proaerolysin molecule to normal cells.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A broad-spectrum anti-cancer agent comprising a modified pore forming protein toxin, said modified pore forming protein toxin derived from a naturally occurring aerolysin-related pore forming protein and comprising a modified activation sequence in which a native protease cleavage site has been functionally deleted and replaced with: one or more general cleavage sites, each cleavable by an enzyme associated with a plurality of cancers, or two or more specific cleavage sites, each cleavable by an enzyme associated with the presence of a specific cancer, wherein cleavage of said modified activation sequence provides an activated pore forming protein toxin capable of killing cancer cells.

2. The broad-spectrum anti-cancer agent according to claim 1, wherein said aerolysin-related pore forming protein toxin is proaerolysin or Clostridium septicum alpha toxin.

3. (canceled)

4. The broad-spectrum anti-cancer agent according to claim 1, wherein said enzyme associated with a plurality of cancers is a protease that is associated with cancer invasion and metastasis, a protease that is up-regulated or secreted by cancer cells, a protease that is activated by enzymes or receptors expressed by cancer cells, or a protease that is associated with angiogenesis.

5. The broad-spectrum anti-cancer agent according to claim 1, wherein said modified activation sequence comprises one or more general cleavage sites cleavable by urokinase-type plasminogen activator or matrix metalloprotease 2.

6-8. (canceled)

9. The broad-spectrum anti-cancer agent according to claim 5, wherein said modified pore forming protein toxin comprises an amino acid sequence at least 90% identical to the sequence as set forth in SEQ ID NO:21 or SEQ ID NO:39.

10-12. (canceled)

13. The broad-spectrum anti-cancer agent according to claim 1, comprising two general cleavage sites, wherein one of said general cleavage sites is cleavable by urokinase-type plasminogen activator, and the other of said general cleavage sites is cleavable by matrix metalloprotease 2.

14. The broad-spectrum anti-cancer agent according to claim 1, wherein said general cleavage site comprises the amino acid sequence SGRSAQ (SEQ ID NO:51).

15. The broad-spectrum anti-cancer agent according to claim 1, wherein said general cleavage site comprises the amino acid sequence HPVGLLAR (SEQ ID NO:52).

16. The broad-spectrum anti-cancer agent according to claim 13, wherein said modified pore forming protein toxin comprises an amino acid sequence at least 90% identical to the sequence as set forth in SEQ ID NO:41.

17. The broad-spectrum anti-cancer agent according to claim 1, wherein said modified pore forming protein toxin further comprises:
a) an artificial regulatory domain capable of targeting said modified pore forming protein toxin to a cell and/or of inhibiting the activity of said modified pore forming protein toxin;
b) one or more mutations in a native binding domain; or
c) a combination of a) and b).

18. The broad-spectrum anti-cancer agent according to claim 17, wherein said artificial regulatory domain is attached to said modified pore forming protein toxin via a linker wherein said artificial regulatory domain is capable of targeting said modified pore forming protein toxin to a cell and/or of inhibiting the activity of said modified pore forming protein.

19. The broad-spectrum anti-cancer agent according to claim 18, wherein said linker comprises an enzyme cleavage site.

20. The broad-spectrum anti-cancer agent according to claim 19, wherein said enzyme cleavage site is cleavable by urokinase-type plasminogen activator, matrix metalloprotease 2, Factor Xa, enterokinase, or thrombin.

21-24. (canceled)

25. The broad-spectrum anti-cancer agent according to claim 17, wherein the targeting unit is an antibody, an antibody fragment, a steroid hormone, a peptide hormone, a neuroactive substance, insulin, a growth factor, a cytokine, melanocyte stimulating hormone, a soluble fragment of CD4, a lectin, an adhesion molecule, a selectin, an integrin, a receptor for an adhesion molecule, a recognition motif for an adhesion molecule, or an enzyme.

26-27. (canceled)

28. The broad-spectrum anti-cancer agent according to claim 17, wherein the artificial regulatory domain is an AFAI antibody fragment.

29. The broad-spectrum anti-cancer agent according to claim 28, wherein said modified pore forming protein toxin comprises an amino acid sequence at least 90% identical to the sequence as set forth in SEQ ID NO:23 or SEQ ID NO:25.

30. (canceled)

31. The broad-spectrum anti-cancer agent according to claim 17, wherein said one or more mutations in a native binding domain are a mutation at position Y61, a mutation at position Y162, a mutation at position W324, a mutation at position R323, a mutation at position R336, a mutation at position W 127, or a combination thereof.

32. The broad-spectrum anti-cancer agent according to claim 31, wherein at least one mutation is R336A or R336c.

33. An isolated polynucleotide encoding the broad-spectrum anti-cancer agent according to claim 1.

34. A vector comprising the polynucleotide according to claim 33, wherein the polynucleotide is operatively linked to one or more expression control sequences.

35. A host cell comprising the vector according to claim 34.

36. A modified pore forming protein toxin derived from proaerolysin and comprising a modified activation sequence in which a native protease cleavage site has been functionally deleted and replaced with one or more general cleavage sites cleavable by urokinase-type plasminogen activator or matrix metalloprotease 2, said modified pore forming protein toxin comprising an amino acid sequence at least 90% identical to the sequence as set forth in any one of SEQ ID NOs: 21, 23, 25, 39, or 41.

37. The broad-spectrum anti-cancer agent according to claim 1, further comprising a pharmaceutically acceptable carrier.

38. The isolated polynucleotide according to claim 33, further comprising a pharmaceutically acceptable carrier.

39-74. (canceled)

75. The method of claim 76, wherein treating cancer comprises decreasing the size of a tumor.

76. A method of treating cancer comprising administering to a subject having cancer an effective amount of the broad-spectrum anti-cancer agent according to claim 1.

77. A method of preparing a broad-spectrum anti-cancer agent, said method comprising:
providing a native pore forming protein toxin wherein said native pore forming protein toxin is proaerolysin or Clostridium septicum alpha toxin; and
modifying the activation sequence of said native pore forming protein toxin such that a native protease cleavage site is functionally deleted and replaced by one or more general cleavage sites each cleavable by an enzyme associated with a plurality of cancers, or by two or more specific cleavage sites each cleavable by an enzyme associated with the presence of a specific cancer, wherein cleavage of said modified activation sequence provides an activated pore forming protein toxin capable of killing cancer cells.

78. The broad spectrum anti-cancer agent of claim 25, wherein the targeting unit is a cytokine.

79. The broad spectrum anti-cancer agent of claim 78, wherein the cytokine is interleukin-2.

80. The broad spectrum anti-cancer agent of claim 78, wherein said aerolysin-related pore forming protein toxin is proaerolysin or Clostridium septicum alpha toxin.

81. The method of claim 76, further comprising administering to the subject a therapeutically effective amount of one or more anti-cancer therapeutics.

82. A method of treating cancer comprising administering to a subject having cancer an effective amount of the broad-spectrum anti-cancer agent according to claim 78.

83. The method of claim 82, wherein the cytokine is interleukin-2.

84. The method of claim 82, wherein the aerolysin-related pore forming protein toxin is proaerolysin or Clostridium septicum alpha toxin.

85. The method of claim 84, further comprising administering to the subject a therapeutically effective amount of one or more anti-cancer therapeutics.
